(19) United States
(12) Patent Application Publication
(10) Pub. No.: US 2025/0250319 A1
(43) Pub. Date: Aug. 7, 2025

(54) CHIMERIC TRANSMEMBRANE POLYPEPTIDES COMPRISING A TGF BETA RECEPTOR EXTRACELLULAR DOMAIN, A TRANSMEMBRANE DOMAIN AND AN INTERLEUKIN RECEPTOR 2 INTRACELLULAR SIGNALLING DOMAIN AND ITS USE IN THERAPY

(71) Applicant: FRED HUTCHINSON CANCER CENTER, Seattle, WA (US)

(72) Inventors: Philip D. GREENBERG, Mercer Island, WA (US); Ashley M. Thelen, Seattle, WA (US)

(21) Appl. No.: 18/578,435

(22) PCT Filed: Jul. 14, 2022
(86) PCT No.: PCT/US2022/073744

§ 371 Date: Jan. 11, 2024

Related U.S. Application Data
(60) Provisional application No. 63/222,400, filed on Jul. 15, 2021.

(51) Int. Cl. C07K 14/71; A61K 38/00; C07K 14/715

(52) U.S. Cl. CPC C07K 14/71; C07K 14/7155; A61K 38/00; C07K 2319/03

(57) ABSTRACT

The present disclosure provides fusion proteins with novel signaling properties. Disclosed embodiments include fusion proteins that comprise an extracellular component that is capable of binding to a TGFβ polypeptide, a transmembrane component, and an intracellular component comprising a cytoplasmic domain from an IL-2Rβ or IL-2Rγ protein. Certain embodiments include a TGFβR intracellular overhang or overhang sequence extending from a TGFβR transmembrane domain, N-terminal to the IL-2R cytoplasmic domain. In response to TGFβ, the fusion proteins can initiate an IL-2 signal in a host cell, promoting, for example, proliferation of the host cells. Recombinant host cells expressing the fusion proteins, and an optional antigen-binding protein such as a TCR or a CAR, are provided. Also provided are polynucleotides encoding the fusion proteins, and vectors that comprise the polynucleotides. Also provided are compositions and methods comprising the same.

### REFERENCE TO AN ELECTRONIC SEQUENCE LISTING

The contents of the electronic sequence listing (360056_494WO_SEQUENCE_LISTING.xml; Size: 83,234 bytes; and Date of Creation: Jul. 13, 2022) is herein incorporated by reference in its entirety.

### BACKGROUND

Adoptive cell therapies for treating cancer and other diseases are still developing, and new strategies are needed, for example, to improve cellular immunotherapy in vivo, such as in the context of solid tumors. The presently disclosed embodiments address these needs and provide other related advantages.

### BRIEF DESCRIPTION OF THE DRAWINGS

FIG. 1 provides schematic illustrations of four expression constructs, each encoding two TGFβR/IL-2R chimeric fusion proteins of the present disclosure. Bicistronic constructs encoding certain chimeric fusion proteins of the present disclosure include a furin protease cleavage site, GSG linker, and P2A self-cleaving peptide sequence separating the cistrons. Further constructs contained the transgenes as shown, with an additional furin protease cleavage site-GSG-E2A downstream of the second fusion protein, followed by a truncated human nerve growth factor receptor (NGFR) as a transduction marker.

FIGS. 2A-3B show IL-2 signaling via pSTAT5 by T cells expressing chimeric fusion proteins and stimulated with TGFβ. (2A, 3A) Primary human CD8 T cells with CRISPR-Cas9-mediated TGFβR1/2 deletions were transduced with constructs encoding chimeric fusion proteins and stimulated with the indicated amounts of recombinant human TGFβ1 or IL-2, followed by fixation/permeabilization and staining with a phospho-specific STAT5 antibody. (2B, 3B) Primary human CD8 T cells with or without TGFβR1/2 deletions were transduced with chimeric constructs and stimulated with 25 ng/mL (2B) or 10 ng/mL (3B) TGFβ1, followed by fixation/permeabilization, and pSTAT5 staining. Modest increases in pSTAT5 signal were observed upon deletion of the endogenous TGFβ receptors 1 and 2. In the graphs.
“1b-2g IL2 TM” indicates a transgene encoding a first fusion containing TGFβR1 extracellular domain, IL-2Rβ transmembrane domain, and IL-2Rβ intracellular domain, and a second fusion containing TGFβR2 extracellular domain, IL-2Rγ transmembrane domain, and IL-2Rγ intracellular domain;
“2b-1g IL2 TM” indicates a transgene encoding a first fusion containing TGFβR2 extracellular domain, IL-2Rβ transmembrane domain, and IL-2Rβ intracellular domain, and a second fusion containing TGFβR1 extracellular domain, IL-2Rγ transmembrane domain, and IL-2Rγ intracellular domain;
“1b-2g TGFbR TM” indicates a transgene encoding a first fusion containing TGFβR1 extracellular domain, TGFβR1 transmembrane domain (followed in the construct by the amino acids C-H-N, in other words, a 3-amino-acid intracellular overhang from TGFβR1), and IL-2Rβ intracellular domain, and a second fusion containing TGFβR2 extracellular domain, TGFβR2 transmembrane domain, and IL-2Rγ intracellular domain; and
“2b-1g TGFbR TM” indicates a transgene encoding a first fusion containing TGFβR2 extracellular domain, TGFβR2 transmembrane domain, and IL-2Rβ intracellular domain, and a second fusion containing TGFβR1 extracellular domain, TGFβR1 transmembrane domain (followed by in the construct by the amino acids C-H-N), and IL-2Rγ intracellular domain.

FIGS. 4A-5B show endogenous TGFβR1/2 signaling in T cells via pSMAD2/3. (4A, 5A) Primary human CD8 T cells (intact TGFβR1/2) were transduced with chimeric constructs and stimulated with the indicated amounts of recombinant human TGFβ1 or IL-2, followed by fixation/permeabilization and staining with a phospho-specific SMAD2/SMAD3 antibody. (4B, 5B) Primary human CD8 T cells with or without TGFβR1/2 deletions (dark and light grey bars, respectively) were transduced with chimeric constructs and stimulated with 25 ng/mL (4B) or 10 ng/mL (5B) TGFβ1, followed by fixation/permeabilization, and pSMAD2/3 staining. The magnitude of pSMAD2/3 signal is reduced in cells transduced with chimeras relative to the untransduced control. Data are from experiments using various concentrations of TGFβ, as shown.

FIGS. 6A and 6B show viability of and transgene expression by T cells transduced with chimeric fusion transgenes and cultured in the presence of TGFβ. Primary human T cells transduced with (6A) TGFβR1-EC_IL-2Rβ-TM_IL-2Rβ-IC/TGFβR2-EC_IL-2Rγ-TM_IL-2Rγ-IC or (6B) TGFβR2-EC_TGFβR2-TM_IL-2Rβ-IC/TGFβR1-EC_TGFβR1-TM (with C-H-N)_IL-2Rγ-IC (multicistronic expression cassettes driven by MSCV U3 promoter with chimeric fusions separated from one another by a furin cleavage site and P2A sequence, the second fusion being separated from NGFRt by a further furin cleavage site and E2A sequence), were cultured in the indicated amount of recombinant human TGFβ1 or IL-2 and re-stimulated with the indicated cytokine condition every 48 hours, up to 7 days. Viability and the percentage of cells expressing the transgenic sequence (as measured by the transduction marker NGFR) were measured over time. Addition of TGFβ1 selected for cells expressing the transgenic constructs (relative to IL-2 or media only controls) and supported IL-2 signal transduction at levels sufficient to maintain viability.

FIGS. 7A and 7B show cell division by T cells expressing chimeric fusions and stimulated with TGFβ1 (7A) or with IL-2 (7B). Primary human CD8 T cells were labeled with CellTrace Violet and stimulated with recombinant human TGFβ1 or IL-2 at the indicated concentrations every 48 hours. After 5 days, cell proliferation (as measured by dilution of CTV signal via flow cytometry) was enhanced by addition of TGFβ1 to cells transduced with the chimeras, but not the untransduced controls. TGFβ1-mediated signaling through the chimeric constructs promotes cell division to a similar extent as IL-2 in the untransduced control cells. Top row of flow images=control; middle row=transduced with construct encoding TGFβR1-EC_IL-2Rβ-TM_IL-2Rβ-IC/TGFβR2-EC_IL-2Rγ-TM_IL-2Rγ-IC; bottom row=transduced with construct containing TGFβR2-EC_IL-2Rβ-TM_IL-2Rβ-IC/TGFβR1-EC_IL-2Rγ-TM_IL-2Rγ-IC.

FIG. 8 shows killing of MDA-MB-468 tumor cell line by primary human T cells that were either (i) transduced to express an exogenous TCR recognizing a WT-1 antigen expressed by the tumor cell line, and stimulated with IL-2, (ii) transduced to express the TCR as well as a chimeric fusion of the present disclosure, and stimulated with TGFβ1, or (iii) transduced to express a chimeric fusion of the present disclosure (but not the TCR), and stimulated with with TGFβ1. Primary human CD8 T cells expressing transgenic constructs were stimulated with the indicated cytokines and co-incubated with NucLight Red+MDA-MB-468 tumors at an effector:target ratio of (Left) 3:1, (Right, Top) 1.5:1, or (Right, Bottom) 0.75:1. Images were acquired every 2 hours (9 images/well, n=3 wells/condition) via the IncuCyte S3 automated imaging system. Fraction of remaining tumor per well (normalized to the first image acquired for each well) was calculated and plotted as a function of time with standard deviation indicated by vertical bars. T cell populations transduced with chimeras and cultured in TGFβ1 cleared tumor with similar magnitude and kinetics as the TCR-only control maintained in IL-2. T cells transduced with TGFβR1-EC_IL-2Rβ-TM_IL-2Rβ-IC/TGFβR2-EC_IL-2Rγ-TM_IL-2Rγ-IC only (no TCR) and cultured in TGFβ1 were included as a negative control.

FIG. 9 shows TGFβR2 expression in unmodified primary human CD8+ T cells and primary human CD8+ T cells with CRISPR-Cas9-mediated knockout of TGFβR2, 72 hours post-knockout via Cas9 RNP electroporation. Guide RNA sequences were: sgTGFβR1: CATACAAACGGCCTATCTCG (SEQ ID NO.:49); and sgTGFβR2: TCACCCGACTTCTGAACGTG (SEQ ID NO.:50). Reagents for validating knockouts were: TGFβR1 fwd genomic primer: AGTGTTTCTGCCACCTCTGT (SEQ ID NO.:51); TGFβR1 rev genomic primer: TGCCTCTAAACGGAATGAGC (SEQ ID NO.:52); Synthego ICE (inference of CRISPR edits) analysis tool (Hsiau et al. bioRxiv 2018 DOI:10.1101/251082); and TGFβR2 APC antibody: Biolegend clone W11755E (catalog #399706).

FIG. 10 summarizes an Alamar blue cell viability assay.

FIGS. 11A-11B show results from the Alamar blue cell viability assay, tested on CTLL-2 cells transduced to express in the indicated chimeric fusion constructs and stimulated with TGFβ (A) or IL-2 (B).

FIGS. 12A-12D show that TGFβR/IL-2R chimeric fusion proteins repurpose TGFβ1 to transmit an IL-2 signal via pSTAT5. (A) Primary human CD8 T cells were activated, transduced with chimeric constructs, and stimulated with the indicated amounts of recombinant human TGFβ1 or IL-2, followed by fixation/permeabilization and staining with a phospho-specific (Y694) STAT5 antibody. The fusions containing TGFβRI transmembrane domain further contained the three-amino-acid intracellular overhang C-H-N extending from the transmembrane sequence. Dots represent individual donors, n=2. (B) Primary human CD8 T cells were electroporated with Cas9 and sgRNA targeting TGFβRII to knock-out endogenous TGFβRII as shown by loss of signal of TGFβRII relative to the no-CRISPR control. The figure key corresponds to the top-to-bottom order of the histogram graphs. (C) Primary human CD8 T cells with CRISPR-Cas9-mediated TGFβRI/II deletions were transduced, stimulated, and analyzed by flow cytometry as described above. (D) Comparison of pSTAT5 signaling between CD8 T cells with intact or CRISPR-mediated deletion of endogenous TGFβRI/II (double knock-out, DKO) from (A) and (C), the 10 ng/mL TGFβ1 stimulation condition is shown here for each of the chimeric constructs and untransduced control. Modest increases in pSTAT5 signal were observed upon deletion of the endogenous TGFβRI/II, suggesting minimal interference of the endogenous receptors with chimeric receptor signal transduction.

FIGS. 13A-13B show that TGFβR/IL-2R chimeric fusion proteins reduce endogenous TGFβRI/II signaling via pSMAD2/3. (A) Primary human CD8 T cells were transduced with chimeric constructs and stimulated with the indicated amounts of recombinant human TGFβ1, followed by fixation/permeabilization and staining with a phospho-specific SMAD2(S465/S467)/SMAD3(S423/S425) antibody. The fusions containing TGFβRI transmembrane domain further contained the three-amino-acid intracellular overhang C-H-N extending from the transmembrane sequence. Dots represent individual donors, n=3. (B) Primary human CD8 T cells with or without TGFβRI/II deletions were transduced, stimulated, and analyzed as described above, the 10 ng/mL TGFβ1 stimulation condition is shown here for each of the chimeric constructs and untransduced control. The magnitude of pSMAD2/3 signal is reduced when endogenous TGFβRI/II are eliminated for all conditions, suggesting that endogenous TGFβ1 signaling is occurring, but to a lesser extent in T cells transduced with TGFβR/IL-2R chimeras (comparing the difference between WT and DKO for untransduced vs. chimeras).

FIG. 14 shows schematic illustrations of two expression constructs, each encoding two TGFβR/IL-2R chimeric fusion proteins of the present disclosure along with a T cell receptor (TCR) specific for a mesothelin antigen peptide. The sequences encoding TCR chains (β, α) and TGFβR/IL-2R chimeric fusion proteins are separated by sequences encoding a furin protease cleavage site, a GSG linker, and a 2A self-cleaving peptide sequence. The fusion containing TGFβRI transmembrane domain further contained the three-amino-acid intracellular overhang C-H-N extending from the transmembrane sequence.

FIGS. 15A-15B show TGFβ1 dose response curves showing differences in pSTAT5 signaling mediated by TGFβR/IL-2R chimeric fusion proteins with a mesothelin-specific TCR. The fusion containing TGFβRI transmembrane domain further contained the three-amino-acid intracellular overhang C-H-N extending from the transmembrane sequence. Primary human CD8 T cells with CRISPR-mediated TGFβRI/II deletion were activated, transduced with chimeric constructs, and stimulated with the indicated amounts of recombinant human TGFβ1, followed by fixation/permeabilization and staining with a phospho-specific (Y694) STAT5 antibody. Results from two independent donors (A, B) are shown.

FIGS. 16A-16B show that culturing chimeric fusion protein- and TCR-transduced T cells in TGFβ1 selects for tetramer expression. Primary human CD8 T cells transduced with (A) mesothelin-specific TCR/TGFβR1-IL-2βTM-IL-2β/TGFβRII-IL2γTM-IL2γ or (B) mesothelin-specific TCR/TGFβRII-TGFβRII TM-IL-2β/TGFβRI-TGFβRI TM-IL-2γ, were cultured in the indicated amount of recombinant human TGFβ1 or IL-2 and re-stimulated with cytokine every 48 hours for up to 7 days. The fusion containing TGFβRI transmembrane domain further contained the three-amino-acid intracellular overhang C-H-N extending from the transmembrane sequence. T cells expressing the transgenic constructs (as measured by mesothelin peptide:MHC tetramer staining) were tracked over time and graphed as percentage of total CD8+ T cells (left) or fold enrichment as calculated by the ratio of tetramer-positive cells in a given condition relative to the 50 U/mL IL-2 control condition (right). These data show that addition of TGFβ1 strongly selects for cells expressing the transgenic constructs.

FIGS. 17A-17E show that mesothelin-specific TCR co-expressed with TGFβR/IL-2R chimeric receptor supports proliferation and tumor killing with TGFβ1 stimulation alone. Primary human CD8 T cells expressing a mesothelin-specific TCR only or the TCR with chimeric fusion proteins as in FIG. 14 were co-incubated with NucLight Red+PANC-1 tumors at an effector:target ratio of (A) 20:1, (B) 10:1, or (C) 5:1. Images were acquired every 2 hours (9 images/well, n=4 wells/condition) via the IncuCyte live cell imaging platform and co-cultures were stimulated with 10 ng/mL human TGFβ1 every 48 hours as indicated by the arrows. The fraction of remaining tumor area per well (normalized to the first image acquired for each individual well) was plotted as a function of time with standard deviation indicated by vertical bars. (D) T cells expressing TCR with chimeric fusion proteins proliferate more than TCR-only T cells cultured in TGFβ L T cell fold-expansion was quantified by dividing the total number of T cells as quantified from each well at the final time point (t=168 hr) divided by the total number of T cells in the first image acquired (t=0), dots represent replicate wells (n=4). (E) All three populations of T cells express similar levels of mesothelin-specific TCR on the surface as quantified by tetramer staining of the T cell populations before co-culture with tumor cells.

FIGS. 18A-18D show that cytotoxicity of T Cells expressing a mesothelin-specific TCR with TGFβR/IL-2R chimeric fusion protein is similar to TCR-only T cells with conventional IL-2 stimulation. Primary human CD8 T cells expressing a mesothelin-specific TCR only or the TCR with chimeric fusion proteins as in FIG. 14 were co-incubated with NucLight Red+PANC-1 tumors at an effector:target ratio of (A) 20:1, (B) 10:1, or (C) 5:1. Images were acquired every 2 hours (9 images/well, n=4 wells/condition) via the IncuCyte live cell imaging platform and co-cultures were stimulated with 50 U/mL IL-2 every 48 hours as indicated by the arrows. The fraction of remaining tumor area per well (normalized to the first image acquired for each individual well) was plotted as a function of time with standard deviation indicated by vertical bars. (D) T cells expressing the TCR with chimeric fusion protein have no proliferative advantage over TCR-only T cells when cultured in IL-2. T cell fold expansion was quantified by dividing the total number of T cells quantified from images of each well at the final time point (t=168 hr) divided by the total number of T cells in the first image acquired (t=0), dots represent replicate wells (n=4).

FIGS. 19A-19D show that proliferation and cytotoxicity of T Cells expressing a mesothelin-specific TCR with chimeric fusion proteins is enhanced by combination treatment of 10 ng/mL TGFβ1 and 5 U/mL IL-2. Primary human CD8 T cells expressing a mesothelin-specific TCR only or the TCR with chimeric fusion proteins as in FIG. 14 were co-incubated with NucLight Red+PANC-1 tumors at an effector:target ratio of (A) 20:1, (B) 10:1, or (C) 5:1. Images were acquired every 2 hours (9 images/well, n=4 wells/condition) via the IncuCyte live cell imaging platform and co-cultures were stimulated with 10 ng/mL TGFβ1 and 5 U/mL IL-2 every 48 hours as indicated by the arrows. The fraction of remaining tumor area per well (normalized to the first image acquired for each individual well) was plotted as a function of time with standard deviation indicated by vertical bars. (D) T cells expressing the TCR with chimeric fusion proteins proliferate more than TCR-only T cells when cultured in a combination of TGFβ1 and IL-2. T cell fold expansion was quantified by dividing the total number of T cells as quantified from each well at the final time point (t=168 hr) divided by the total number of T cells in the first image acquired (t=0), dots represent replicate wells (n=4).

FIGS. 20A-20B show that addition of 3 amino acids from the TGFβR1 intracellular sequence proximal to the transmembrane domain promotes IL-2R signaling. (A) Primary human CD8 T cells were activated, transduced with chimeric constructs, and stimulated with the indicated amounts of recombinant human TGFβ1 or IL-2, followed by fixation/permeabilization and staining with a phospho-specific (Y694) STAT5 antibody. Dots represent individual donors, n=2. (B) Primary human CD8 T cells were transduced with chimeric constructs and stimulated with the indicated amounts of recombinant human TGFβ1, followed by fixation/permeabilization and staining with a phospho-specific SMAD2(S465/S467)/SMAD3(S423/5425) antibody. Dots represent individual donors, n=3. The construct 2β-1γ (TGFβR TM) contains 3 amino acids from TGFβR1 proximal to the transmembrane region (C-H-N), while the other construct (2β-1γ (TGFβR TM-3AA)) contains amino acids from the extracellular and transmembrane regions of TGFβRI, but does not contain the TGFβRI intracellular overhang; in other words, the 3-amino acid intracellular overhang was removed.

FIGS. 21-26C relate to experiments testing murine versions of chimeric fusion proteins.

FIG. 21 shows schematic illustrations of two expression constructs, each encoding two murine versions of a TGFβR/IL-2R chimeric fusion protein of the present disclosure, along with TCR1045, isolated from MSLN^{-/-} mice and specific for a mesothelin antigen peptide (see Stromnes et al. Cancer Cell 28(5):638-685 (2015), TCR1045 is incorporated herein by reference). The construct design is generally similar to that shown and described for FIG. 14, though the fusion protein containing murine TGFβRI transmembrane domain did not contain the C-H-N intracellular overhang (conserved between mouse and human TGFβRI). The retroviral backbone pMP71 was used for delivery of the constructs to T cells.

FIG. 22 shows that murine TGFβR/IL-2R chimeras convert mouse TGFβ1 to an IL-2 signal via pSTAT5. Primary mouse P14 splenocytes were activated, transduced with chimeric constructs, and stimulated with the indicated amounts of recombinant murine TGFβ1 or human IL-2, followed by fixation/permeabilization and staining with a phospho-specific (Y694) STAT5 antibody. Dots represent biological replicates, n=3. Events shown are gated on live, Vβ9-positive (Msln 1045 TCR beta chain) cells.

FIGS. 23A-23B show that culturing murine T cells in TGFβ1 selects for tetramer expression. Murine CD8 T cells transduced with (A) Msln1045 TCR/TGFβRI-IL2βTM-IL2β/TGFβRII-IL2γTM-IL2γ or (B) Msln1045 TCR/TGFβRII-TGFβRII TM-IL2β/TGFβRI-TGFβRI TM-IL2γ, were cultured in the indicated amount of recombinant mouse TGFβ1 or human IL-2 and re-stimulated with cytokine every 48 hours for up to 7 days. T cells expressing the transgenic constructs (as measured by staining with tetramer and an antibody recognizing the Msln1045 TCR beta chain, Vβ9) were tracked over time and graphed as percentage of total CD8+ T cells. These data show that addition of TGFβ1 strongly selects for cells expressing the transgenic constructs.

FIGS. 24A-24C relate to murine therapeutic T cell production and characterization. (A) Diagram of murine Msln 1045 TCR constructs with and without murine TGFβR/IL-2R chimeras. Multicistronic constructs contained furin protease cleavage site, GSG linker, and 2A self-cleaving peptide sequence between each protein (TCR or chimeric fusion)-encoding sequence. (B) Experimental workflow for engineering therapeutic murine T cells. Splenocytes are harvested from transgenic P14 donor mice which are either Thy1.2 homozygous/CD45.1 negative or Thy1.2/Thy1.1 heterozygous/CD45.1 positive for TCR only and TCR with chimeric fusion proteins, respectively, and activated with plate-bound anti-CD3/anti-CD28 antibody, IL-2, and IL-21. Activated T cells are transduced with retrovirus and after 7 days the transduced cells are expanded by co-culture with irradiated antigen-presenting cells pulsed with the cognate Msln peptide and IL-2. (C) One week after expansion, engineered donor cells were analyzed by flow cytometry for expression of the therapeutic TCR by surface staining with Msln:HLA tetramer and an antibody that binds to the Msln 1045 TCR beta chain (Vβ9), as shown by dot plots on right. All plots were gated on live, Thy1.2-positive cells. The genotype of donor P14 splenocytes for each condition is shown by the dot plots on the left, staining for the congenic markers Thy1.1 and CD45.1.

FIGS. 25A-25B show Msln 1045 TCR with TGFβR/IL-2R chimeras as TCR-T cell therapy for Pancreatic Ductal Adenocarcinoma (PDA) in a genetically engineered KrasLSL-G12D/+; Trp53LSL-R172H/+;p48Cre/+(KPC) mouse model. (A) Adoptive cell transfer (ACT) workflow for treatment of tumor bearing KPC mice. KPC mice were screened via ultrasound imaging for evidence of PDA and subsequently enrolled into one of three treatment groups shown (n=5 mice, 2/group for co-transfers). Recipient mice received cytoxan as pre-conditioning therapy prior to adoptive transfer of engineered T cells along with IL-2 and irradiated antigen presenting cells (APC) pulsed with the cognate Msln peptide. For the co-transfer groups 1 and 2, the TCR-with-chimera T cells were transferred at approximately a 1:1 ratio. The total number of transferred cells per mouse for each group is shown in (B).

FIGS. 26A-26C show that engineered T cells containing TGFβR/IL-2R chimeras preferentially accumulate in pancreatic tumors relative to Msln 1045 TCR-only T cells. (A) KPC mice were treated with the indicated engineered T cells one week prior to harvest. Pancreatic tumor tissue was dissociated into a single cell suspension and analyzed by flow cytometry to measure the relative ratio of co-transferred T cell populations. (B) Diagram indicating congenic markers corresponding to the three populations of T cells harvested from tumors based on the genetic background of the donor and recipient T cells. (C) Direct comparison of co-transferred T cell populations shows that T cells engineered to express the Msln 1045 TCR and the TGFβR/IL-2R chimeric fusion proteins (top, “Group 1”; bottom “Group 2”) preferentially accumulate in tumors relative to T cells expressing the Msln1045 TCR only (comparing Thy1. 1 negative/CD45.1 positive cells to Thy1.1 positive/CD45.1 negative cells, respectively). All dot plots were gated on live, Thy 1.2 positive cells. Each plot represents one mouse and treatment groups correspond to groups shown in (A). The observed abundance of endogenous T cells (Thy1.1 negative/CD45.1 negative population) in the tumor for all mice may be due to inclusion of draining lymph nodes upon dissection of the pancreatic tumor tissue, as has been reported for the KPC model (see Lee et al., Curr Protoc Pharmacol. 2016; 73:14.39.1-14.39.20. Published 2016 Jun. 1. doi:10.1002/cpph.2), where tumor can readily grow into nearby lymph nodes making it difficult to distinguish lymph nodes from the tumor.

### DETAILED DESCRIPTION

The present disclosure provides, in part, fusion proteins (also referred to herein as “chimeric” fusion proteins or “chimeras”) that are capable of binding to a transforming growth factor-beta (TGFβ) polypeptide (or a dimer thereof) and thereby contributing to interleukin-2 (IL-2) signaling in a host cell. In other words, certain embodiments provide fusion proteins that convert a TGFβ input to an IL-2 signal in a host cell.

In the present disclosure, it will be understood that TGFβR1 can also be referred-to as TGFβR1, as TGFbR1, or as TGFbRI. It will be understood that TGFβR2 can also be referred-to as TGFβRII, as TGFbR2, or as TGFbRII. It will be understood that IL-2Rγ can also be referred-to as IL2-Rg, as IL2Rg, or as IL2Rγ. It will be understood that IL-2Rβ can also be referred-to as IL2-Rb, as IL2Rb, or as IL2Rβ.

By way of background, under normal conditions, binding of a TGFβ receptor (TGFβR) complex to TGFβ delivers a suppressive signal to cells that is counter to the promotive (e.g., promoting differentiation, proliferation, effector function, and nutrient uptake) signaling initiated by binding of an IL-2R complex to IL-2. Briefly, binding of a TGFβ1 homodimer to TGFβR2 promotes assembly of a heterotetrameric receptor complex comprised of two TGFβR1 proteins and two TGFβR2 proteins, forming a symmetric 2:2:2 ligand-receptor complex. Ligand binding promotes intracellular serine/threonine kinase activity of TGFβR1, which phosphorylates SMAD2/SMAD3 proteins, leading to their nuclear translocation and association with transcription factors to modulate transcriptional responses. TGFβ1 signaling results in reduced proliferation and effector function of T cells. IL-2 binding to a high-affinity receptor (IL-2R) complex containing alpha (α), beta (β), and gamma (γ) chains results in recruitment of JAK1 and JAK3 tyrosine kinases to IL-2Rβ and IL-2Rγ, respectively. Upon activation, JAK1/3 phosphorylate IL-2Rβ, leading to recruitment of STAT5 and its subsequent phosphorylation and activation. Phosphorylated STAT5 homodimers translocate to the nucleus to drive transcription of genes involved in T cell differentiation and effector function. IL-2 signaling also leads to activation of MAP kinases and mTORC1 to drive T cell proliferation and nutrient uptake. Overall, TGFβ1 and I1-2 signaling pathways exert opposite effects on T cells.

Plasma levels of TGFβ1 have been correlated with the extent of disease in colorectal cancer and reported as predictive of liver metastasis in after curative resection for colorectal cancer (Tsushima et al., Clin. Cancer Res. 7:1258-1262 (2001)); pretreatment levels of soluble TGFβ have also been reported as prognostic indicators in pancreatic cancer settings (Park et al., Cancer Medicine 9:43-51 (2020) doi: 10.1002/cam4.2677)).

Certain embodiments of presently disclosed fusion proteins comprise an extracellular component that is capable of binding to a TGFβ polypeptide (e.g., a TGFβ1 homodimer), a transmembrane component, and an intracellular component comprising an intracellular portion from an IL-2Rβ or an IL-2Rγ. The extracellular component can comprise all or a portion of, or can be derived from, an extracellular domain of a TGFβR polypeptide (e.g., TGFβR1 or TGFβR2). The transmembrane component can comprise all or a portion of, or can be derived from, a TGFβR polypeptide or an IL-2R polypeptide.

In some embodiments, the intracellular component comprises, extending from a TGFβR transmembrane domain of the fusion protein and N-terminal to the intracellular portion from an IL-2Rβ or an IL-2Rγ, one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, for, five, six, seven, eight, nine, ten, eleven, twelve, thirteen, fourteen, fifteen, sixteen, seventeen, eight, nineteen, or twenty consecutive amino acids from the intracellular domain of the TGFβR (source) polypeptide, or a variant thereof comprising one, two, three, four, or five, optionally conservative, amino acid substitutions; these amino acid(s) are preferably from the N-terminal portion of the TGFβR source polypeptide intracellular domain. In other words, certain embodiments comprise a portion of a TGFβR polypeptide that comprises a TGFβR transmembrane domain and N-terminal portion of the TGFβR intracellular domain, or a variant of the N-terminal portion comprising one, two, three, four, or five, optionally conservative, amino acid substitutions. Such an intracellular amino acid or amino acid sequence (i.e., an N-terminal, membrane-proximal amino acid or amino acid sequence of a fusion protein intracellular component) from the same TGFβR source polypeptide contributing to the transmembrane component of the fusion protein, or a variant thereof comprising one, two, three, four, or five, optionally conservative, amino acid substitutions) can be referred-to as an intracellular overhang or intracellular overhang sequence.

For example, in some embodiments, the transmembrane component of a fusion protein comprises a TGFβRI transmembrane domain and the intracellular component of the fusion protein comprises, at its N-terminal end, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal amino acids from a TGFβRI intracellular domain, or a variant thereof comprising one, two, three, four, or five, optionally conservative, amino acid substitutions; by way of illustration, the N-terminal twenty amino acids of a human TGFβRI intracellular domain are shown in bold, underlined font below:

(SEQ ID NO.: 1)
MEAAVAAPRP RLLLLVLAAA AAAAAALLPG ATALQCFCHL CTKDNFTCVT
DGLCFVSVTE TTDKVIHNSM CIAEIDLIPR DRPFVCAPSS KTGSVTTTYC
CNQDHCNKIE LPTTVKSSPG LGPVELAAVI AGPVCFVCIS LMLMVYICHN
RTVIHHRVPN EEDPSLDRPF ISEGTTLKDL IYDMTTSGSG SGLPLLVQRT
IARTIVLQES IGKGRFGEVW RGKWRGEEVA VKIFSSREER SWFREAEIYQ
TVMLRHENIL GFIAADNKDN GTWTQLWLVS DYHEHGSLED YLNRYTVTVE
GMIKLALSTA SGLAHLHMEI VGTQGKPAIA HRDLKSKNIL VKKNGTCCIA
DLGLAVRHDS ATDTIDIAPN HRVGTKRYMA PEVLDDSINM KHFESFKRAD
IYAMGLVFWE IARRCSIGGI HEDYQLPYYD LVPSDPSVEE MRKVVCEQKL
RPNIPNRWQS CEALRVMAKI MRECWY ANGA ARLTALRIKK TLSQLSQQEG IKM
CHN RTVIHHRVPN EEDPSLDRPF ISEGTTLKDL IYDMTTSGSG SGLPLLVQRT
IARTIVLQES IGKGRFGEVW RGKWRGEEVA VKIFSSREER SWFREAEIYQ
TVMLRHENIL GFIAADNKDN GTWTQLWLVS DYHEHGSLFD YLNRYTVTVE
GMIKLALSTA SGLAHLHMEI VGTQGKPAIA HRDLKSKNIL VKKNGTCCIA
DLGLAVRHDS ATDTIDIAPN HRVGTKRYMA PEVLDDSINM KHFESFKRAD
IYAMGLVFWE IARRCSIGGI HEDYQLPYYD LVPSDPSVEE MRKVVCEQKL
RPNIPNRWQS CEALRVMAKI MRECWYANGA ARLTALRIKK TLSQLSQQEG IKM

Accordingly, in some embodiments, a fusion protein comprises a TGFβRI transmembrane domain and extending from the transmembrane domain into the intracellular component of the fusion protein is the first 1, 2, 3, 4, 5, 6, 7, 8, 9, 10, 11, 12, 13, 14, 15, 16, 17, 18, 19, or 20 amino acids shown in bold, underlined font above, or a variant thereof comprising one, two, three, four, or five, optionally conservative, amino acid substitutions.

As another example, in some embodiments, the transmembrane component of a fusion protein comprises a TGFβ3RII transmembrane domain and the intracellular component of the fusion protein comprises, at its N-terminal end, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal amino acids from a TGFβRII intracellular domain, or a variant thereof comprising one, two, three, four, or five, optionally conservative, amino acid substitutions; by way of illustration, the N-terminal twenty amino acids of a human TGFβRII intracellular domain are shown in bold, underlined font below:

(SEQ ID NO.: 2)
MGRGLLRGLW PLHIVLWTRI ASTIPPHVQK SVNNDMIVTD NNGAVKFPQL
CKFCDVREST CDNQKSCMSN CSITSICEKP QEVCVAVWRK NDENITLETV
CHDPKLPYHD FILEDAASPK CIMKEKKKPG ETFFMCSCSS DECNDNIIFS
EEYNTSNPDL LLVIFQVTGI SLIPPLGVAI SVIIIFYCYR VNRQQKLSST
WETGKTRKLM EFSEHCAIIL EDDRSDISST CANNINHNTE LLPIELDTLV
GKGRFAEVYK AKLKQNTSEQ FETVAVKIFP YEEY ASWKTE KDIFSDINLK
HENILQFLTA EERKTELGKQ YWLITAFHAK GNLQEYLTRH VISWEDLRKL
GSSLARGIAH LHSDHTPCGR PKMPIVHRDL KSSNILVKND LTCCLCDFGL
SLRLDPTLSV DDLANSGQVG TARYMAPEVL ESRMNLENVE SFKQTDVYSM
ALVLWEMTSR CNAVGEVKDY EPPFGSKVRE HPCVESMKDN VLRDRGRPEI
PSFWLNHQGI QMVCETLTEC WDHDPEARLT AQCVAERFSE LEHLDRLSGR
SCSEEKIPED GSLNTTK

Accordingly, in some embodiments, a fusion protein comprises a TGFβRII transmembrane domain and extending from the transmembrane domain into the intracellular component of the fusion protein is the first 1, 2, 3, 4, 5, 6, 7, 8, 9, 10, 11, 12, 13, 14, 15, 16, 17, 18, 19, or 20 amino acids shown in bold, underlined font above, or a variant thereof comprising one, two, three, four, or five, optionally conservative, amino acid substitutions.

In some embodiments, fusion proteins comprising such an intracellular overhang or overhang sequence provide improved IL-R signaling relative to a reference fusion protein that is otherwise the same but does not comprise the intracellular overhang or overhang sequence. In some embodiments, an intracellular overhang from TGFβR1 comprises or consists of the sequence C-H-N. In some embodiments, an intracellular overhang from TGFβRII comprises or consists of the sequence R-V-N.

Binding by a disclosed fusion protein (e.g., in a complex as described herein and expressed in a host cell) to a TGFβ (e.g., TGFβ1) polypeptide as disclosed herein converts the normally suppressive TGFβ signal into a beneficial IL-2 signal that supports cell activity, such as proliferation and enhanced effector function in T cells.

In certain embodiments, the TGFβR1, the TGFβR2, the IL-2Rγ, and/or the IL-2Rβ are human.

Human TGFβR1 isoforms include the isoforms described in: UniProt KB P36897-1 (“Isoform 1”); UniProt KB P36897-2 (“Isoform 2”); and UniProt KB P36897-3 (“Isoform 3”). Amino acid sequence(s) from any of these isoforms can be present in a disclosed fusion protein; in some embodiments, Isoform 1 is preferred.

Human TGFβR2 isoforms include the isoforms described in: UniProt KB P37173-1 (“Isoform 1”); and UniProt KB P37173-2 (“Isoform 2”). Amino acid sequence(s) from either of these isoforms can be present in a disclosed fusion protein; in some embodiments, Isoform 1 is preferred.

Human IL-2Rγ isoforms include the isoforms described in: UniProt KB P31785-1 (“Isoform 1”); and UniProt KB P31785-2 (“Isoform 2”). Amino acid sequence(s) from either of these isoforms can be present in a disclosed fusion protein; in some embodiments, Isoform 1 is preferred.

Human IL-2Rβ is described in UniProt KB P14784.

Functional variants (as described herein) of amino acid sequences from human TGFβR1, TGFβR2, IL-2Rγ, and/or IL-2Rβ are also contemplated.

It will be understood that by “capable of binding to a TGFβ polypeptide” a disclosed fusion protein comprises structure that allows the fusion protein to bind to a TGFβ polypeptide; typically, binding occurs when a TGFβ dimer, such as a TGFβ1 homodimer, binds to a protein complex comprising two TGFβR1 ligand-binding portions and two TGFβR2 ligand-binding portions. Accordingly, disclosed fusion proteins are “capable of” binding to a TGFβ polypeptide at least in the context of a protein complex that comprises two TGFβR1 ligand-binding portions and two TGFβR2 ligand-binding portions. In other words, “capable of binding” encompasses contributing to binding as part a TGFβ binding complex. Typically, a TGFβ polypeptide will be comprised in a dimer; binding interaction between a fusion protein of the binding complex and a TGFβ polypeptide dimer may comprise interaction between the fusion protein and one or both TGFβ polypeptides of the dimer. In some embodiments, when a fusion protein is present outside the context of such a complex (e.g. as a monomer), binding interaction(s) between the fusion protein and a TGFβ polypeptide (or dimer thereof) may also occur. In some embodiments, a fusion protein is expressed at the surface of a host cell and associates with at least one other fusion protein of the present disclosure, e.g. a fusion protein comprising a TGFβRI extracellular domain associates with a fusion protein comprising a TGFβRII extracellular domain. In some embodiments, a host cell expresses a protein complex comprising two molecules of a fusion protein comprising a TGFβRI extracellular domain and two molecules of a fusion protein comprising a TGFβRII extracellular domain, and the protein complex is capable of binding to a TGFβ molecule or dimer thereof.

A single fusion protein comprising an intracellular portion from IL-2Rβ or IL-2Rγ (or a functional variant or fragment thereof) is capable of at least contributing to IL-2 signaling (e.g., participating in initiation of IL-2 signaling following binding by the fusion protein to a TGFβ polypeptide). As discussed above, under normal conditions, IL-2 signaling typically involves one IL-2Rγ protein and one IL-2Rβ protein (these forming an IL-2 binding complex with an IL-2Rα protein). While one fusion protein comprising IL-2Rγ sequence in its intracellular component and one fusion protein comprising IL-2Rβ sequence in its intracellular component may thus together be sufficient to initiate IL-2 signaling upon appropriate stimulation via ligand-binding, in some embodiments, four fusion proteins (these collectively providing two TGFβR1 ligand-binding portions and two TGFβR2 ligand-binding portions) may associate to form a TGFβ-binding complex, and each of the four fusion proteins may comprise an intracellular component from IL-2Rγ or IL-2Rβ, provided that in the complex, at least one IL-2Rγ intracellular component (or a functional variant or portion thereof) and at least one IL-2Rβ intracellular component (or a functional variant or portion thereof) is present.

In some embodiments, two presently disclosed fusion proteins can be the same as one another or can be different from one another, and can be both expressed by a host cell. In some embodiments, the two fusion proteins can associate at a host cell surface to contribute to a complex that (i) is capable of binding to a TGFβ polypeptide, for example a TGFβ1 polypeptide or homodimer thereof, and (ii) is capable of contributing to an IL-2 signal in the host cell. As a non-limiting illustration, a first fusion protein can comprise an extracellular domain from one of TGFβRI and TGFβR2, and a second fusion protein can comprise an extracellular domain from the other of TGFβR1 and TGFβR2. The first fusion protein can comprise an intracellular domain from one of IL-2Rγ and IL-2Rβ, and the second fusion protein can comprise an intracellular domain from the other of IL-2Rγ and IL-2Rβ. Thus, collectively, the first fusion protein and the second fusion protein in this illustrative embodiment comprise an extracellular domain from TGFβR1, an extracellular domain from TGFβR2, an intracellular domain from IL-2Rγ, and an intracellular domain from IL-2Rβ.

In certain embodiments, four fusion proteins, each of which may be different from the others, or may be the same as one of the others, can associate at a host cell surface to form a complex that is capable of binding to a TGFβ polypeptide (e.g., a TGFβ1 homodimer) and thereby initiate an IL-2 signal in the host cell. Two of the four fusion proteins can comprise a TGFβRI extracellular domain or a portion or variant thereof, and the other two of the four fusion proteins can comprise a TGFβR2 extracellular domain or a portion or variant thereof. In some embodiments, at least one, and preferably two, of the four fusion proteins comprise an intracellular portion of an IL-2Rβ. In certain embodiments, at least one, and preferably two, of the four fusion proteins comprise an intracellular portion of an IL-2Rγ.

In some embodiments, a protein complex comprises a first and a second presently disclosed fusion protein, and binding of a TGFβ polypeptide (or dimer thereof) to the protein complex initiates an IL-2 signal in a host cell expressing the protein complex. In some embodiments, the first fusion protein comprises a TGFβRI extracellular domain or a portion or variant thereof that is functional to bind the TGFβ polypeptide (or dimer thereof), and the second fusion protein comprises a TGFβR2 extracellular domain or a portion or variant thereof that is functional to bind the TGFβ polypeptide (or dimer thereof). In certain further embodiments: (i) the first fusion protein further comprises an IL-2Rβ intracellular portion and the second fusion protein further comprises an IL-2Rγ intracellular portion; or (ii) the first fusion protein further comprises an IL-2R intracellular portion and the second fusion protein further comprises an IL-2Rβ intracellular portion. In some embodiments, the transmembrane component of a fusion protein is from the TGFβR protein from which the extracellular component of the fusion protein is derived, or the transmembrane component of a fusion protein is from the IL-2R protein from which the intracellular component of the fusion protein is derived.

Certain non-limiting embodiments of fusion proteins are summarized in Table A, and certain non-limiting embodiments of first and second fusion proteins (e.g., pairs that can be encoded by a same polynucleotide molecule or vector, and/or can be expressed by a same host cell) are summarized in Table B.

TABLE A
Certain non-limiting fusion protein embodiments

| | | | I.C. | |
|---|---|---|---|---|
| | | | Optional | Intracellular portion (e.g. |
| Fusion | | | intracellular | cytoplasmic/signaling |
| Protein | E.C. | T.C. | overhang^{§} | domain from) |
| “1” | TGFβR1 | TGFβR1 | TGFβR1 | IL-2Rβ |
| “2” | TGFβR1 | IL-2Rβ | — | IL-2Rβ |
| “3” | TGFβR1 | TGFβR1 | TGFβR1 | IL-2Rγ |
| “4” | TGFβR1 | IL-2Rγ | — | IL-2Rγ |
| “5” | TGFβR2 | TGFβR2 | TGFβR2 | IL-2Rβ |
| “6” | TGFβR2 | IL-2Rβ | — | IL-2Rβ |
| “7” | TGFβR2 | TGFβR2 | TGFβR2 | IL-2Rγ |
| “8” | TGFβR2 | IL-2Rγ | — | IL-2Rγ |

E.C. = extracellular component;
T.C. = transmembrane component;
I.C. = intracellular component

In some embodiments, a fusion protein is provided that comprises: (i) an extracellular component comprising an extracellular domain from human TGFβRI; (ii) a transmembrane component comprising a transmembrane domain from human TGFβRI; and (iii) an intracellular component comprising a cytoplasmic/signaling domain from human IL-2Rβ, wherein, optionally, the intracellular component further comprises, disposed N-terminal to the human IL-2Rβ cytoplasmic/signaling domain, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal amino acids from TGFβRI, or a variant thereof comprising one, two, three, four, or five, optionally conservative, amino acid substitutions. In some embodiments, the intracellular component comprises the three amino acids C-H-N disposed N-terminal to the human IL-2Rβ cytoplasmic/signaling domain. ^{§} the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal intracellular amino acids from the TGFβR protein contributing to the transmembrane component, or a variant thereof comprising one, two, three, four, or five, optionally conservative, amino acid substitutions.

In some embodiments, a fusion protein is provided that comprises: (i) an extracellular component comprising an extracellular domain from human TGFβRI; (ii) a transmembrane component comprising a transmembrane domain from human TGFβRI; and (iii) an intracellular component comprising a cytoplasmic/signaling domain from human IL-2Rγ, wherein, optionally, the intracellular component further comprises, disposed N-terminal to the human IL-2Rγ cytoplasmic/signaling domain, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal amino acids from TGFβRI, or a variant thereof comprising one, two, three, four, or five, optionally conservative, amino acid substitutions. In some embodiments, the intracellular component comprises the three amino acids C-H-N disposed N-terminal to the human IL-2Rγ cytoplasmic/signaling domain.

In some embodiments, a fusion protein is provided that comprises: (i) an extracellular component comprising an extracellular domain from human TGFβRI; (ii) a transmembrane component comprising a transmembrane domain from human IL-2Rβ and (iii) an intracellular component comprising a cytoplasmic/signaling domain from human IL-2Rβ.

In some embodiments, a fusion protein is provided that comprises: (i) an extracellular component comprising an extracellular domain from human TGFβRI; (ii) a transmembrane component comprising a transmembrane domain from human IL-2Rγ; and (iii) an intracellular component comprising a cytoplasmic/signaling domain from IL-2Rγ.

In some embodiments, a fusion protein is provided that comprises: (i) an extracellular component comprising an extracellular domain from human TGFβRII; (ii) a transmembrane component comprising a transmembrane domain from human TGFβRII; and (iii) an intracellular component comprising a cytoplasmic/signaling domain from human IL-2Rβ, wherein, optionally, the intracellular component further comprises, disposed N-terminal to the human IL-2Rβ cytoplasmic/signaling domain, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal amino acids from TGFβRII, or a variant thereof comprising one, two, three, four, or five, optionally conservative, amino acid substitutions. In some embodiments, the intracellular component comprises the three amino acids R-V-N disposed N-terminal to the human IL-2Rβ cytoplasmic/signaling domain.

In some embodiments, a fusion protein is provided that comprises: (i) an extracellular component comprising an extracellular domain from human TGFβRII; (ii) a transmembrane component comprising a transmembrane domain from human TGFβRII; and (iii) an intracellular component comprising a cytoplasmic/signaling domain from human IL-2Rγ, wherein, optionally, the intracellular component further comprises, disposed N-terminal to the human IL-2Rγ cytoplasmic/signaling domain, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal amino acids from TGFβRII, or a variant thereof comprising one, two, three, four, or five, optionally conservative, amino acid substitutions. In some embodiments, the intracellular component comprises the three amino acids R-V-N disposed N-terminal to the human IL-2Rγ cytoplasmic/signaling domain.

In some embodiments, a fusion protein is provided that comprises: (i) an extracellular component comprising an extracellular domain from human TGFβRII; (ii) a transmembrane component comprising a transmembrane domain from human IL-2Rβ and (iii) an intracellular component comprising a cytoplasmic/signaling domain from human IL-2Rβ.

In some embodiments, a fusion protein is provided that comprises: (i) an extracellular component comprising an extracellular domain from human TGFβRII; (ii) a transmembrane component comprising a transmembrane domain from human IL-2Rγ; and (iii) an intracellular component comprising a cytoplasmic/signaling domain from IL-2Rγ.

TABLE B
Certain non-limiting fusion protein pairs, with reference to Table A

| First Fusion Protein | Second Fusion Protein |
|---|---|
| “1” | “5” |
| “1” | “6” |
| “1” | “7” |
| “1” | “8” |
| “2” | “5” |
| “2” | “6” |
| “2” | “7” |
| “2” | “8” |
| “3” | “5” |
| “3” | “6” |
| “3” | “7” |
| “3” | “8” |
| “4” | “5” |
| “4” | “6” |
| “4” | “7” |
| “4” | “8” |
| “5” | “1” |
| “5” | “2” |
| “5” | “3” |
| “5” | “4” |
| “6” | “1” |
| “6” | “2” |
| “6” | “3” |
| “6” | “4” |
| “7” | “1” |
| “7” | “2” |
| “7” | “3” |
| “7” | “4” |
| “8” | “1” |
| “8” | “2” |
| “8” | “3” |
| “8” | “4” |

In particular embodiments, a polynucleotide or vector is provided that encodes, or a host cell is provided that expresses: (1) a first fusion protein comprising (1a) an extracellular component comprising an extracellular domain from (e.g. human) TGFβR1, (1b) a transmembrane component comprising a transmembrane domain from (e.g. human) IL-2Rβ, and (1c) an intracellular component comprising a cytoplasmic/signaling domain from (e.g. human) IL-2Rβ; and (2) a second fusion protein comprising (2a) an extracellular component comprising an extracellular domain from (e.g. human) TGFβR2, (1b) a transmembrane component comprising a transmembrane domain from (e.g. human) IL-2Rγ, and (1c) an intracellular component comprising a cytoplasmic/signaling domain from (e.g. human) IL-2Rγ. In some embodiments, the polynucleotide or vector further encodes, or the host cell further expresses, an antigen-binding protein such as a TCR or a CAR.

In other embodiments, a polynucleotide or vector is provided that encodes, or a host cell is provided that expresses: (1) a first fusion protein comprising (1a) an extracellular component comprising an extracellular domain from (e.g. human) TGFβR2, (1b) a transmembrane component comprising a transmembrane domain from (e.g. human) TGFβR2, and (1c) an intracellular component comprising a cytoplasmic/signaling domain from (e.g. human) IL-2Rβ and optionally comprising N-terminal thereto a TGFβR2 intracellular overhang sequence as described for Table A, optionally comprising the sequence R-V-N disposed N-terminal to the IL-2Rβ cytoplasmic/signaling domain; and (2) a second fusion protein comprising (2a) an extracellular component comprising an extracellular domain from (e.g. human) TGFβR1, (1b) a transmembrane component comprising a transmembrane domain from (e.g. human) TGFβR1, and (1c) an intracellular component comprising a cytoplasmic/signaling domain from (e.g. human) IL-2Rγ, and optionally comprising N-terminal thereto a TGFβR1 intracellular overhang or overhang sequence as described in Table A, further optionally wherein the intracellular component comprises the amino acids C-H-N disposed N-terminal to the IL-2Rγ cytoplasmic/signaling domain. In some embodiments, the polynucleotide or vector further encodes, or the host cell further expresses, an antigen-binding protein such as a TCR or a CAR.

Also provided are polynucleotides that encode a fusion protein or fusion proteins as disclosed herein, vectors that comprise a polynucleotide, host cell compositions, and other reagents useful, for example, in treating a disease or disorder such as a cancer. Related methods and uses are also provided.

Disclosed fusion proteins are useful in, for example, cellular immunotherapies, such as for expression by immune cells that also express an antigen-binding protein, such as a TCR, a CAR, or the like. Such host cells may comprise improved persistence, proliferation, effector function, or any combination thereof, as compared to a reference host cell that does not express the fusion protein. For example, in some contexts (e.g., solid tumors), levels of TGFβ may be high (e.g. as compared to serum levels of TGFβ from a healthy individual) and may exert a suppressive effect on cells that do not express a fusion protein or fusion proteins as provided herein. Cells expressing disclosed fusion proteins convert an otherwise suppressive TGFβ signal to an IL-2 signal.

Prior to setting forth this disclosure in more detail, it may be helpful to an understanding thereof to provide additional definitions of certain terms to be used herein. Still more definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. “About” includes ±15%, ±10%, and ±5%. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a protein domain, linker, signal peptide) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or E); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in e.g. Creighton (1984) Proteins, W.H. Freeman and Company, and in Creighton, Proteins: Structures and Molecular Properties (W H Freeman & Co.; 2nd edition (December 1993)).

Variant proteins, peptides, polypeptides, and amino acid sequences of the present disclosure can, in certain embodiments, comprise one or more conservative substitutions relative to a reference amino acid sequence.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

As used herein, “fusion protein” refers to a protein that, in a single chain, has at least two distinct domains and/or motifs, wherein the domains or motifs are not naturally found together (e.g., in the given arrangement, order, or number, or at all) in a protein. In certain embodiments, a fusion protein comprises at least two distinct domains and/or motifs that are not found together in a single naturally occurring peptide or polypeptide. In certain embodiments, a fusion protein comprises amino acid sequences from two or more distinct polypeptides; e.g., a fusion protein can comprise an amino acid sequence or domain from a TGFβR polypeptide and an amino acid sequence or domain from an IL-2R polypeptide. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a fusion protein expressed or produced by a host cell (e.g., a T cell) locates to the cell surface, where the fusion protein can be anchored to the cell membrane.

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. If single-stranded, the nucleic acid molecule may be the coding strand or non-coding (anti-sense strand). A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. In some embodiments, a composition of the present disclosure can be “isolated” in the sense that it is physically separated from and not comprised within a subject to whom the composition can be, was, or is to be administered. Any of the presently disclosed fusion proteins, polynucleotides, vectors, or host cells can be provided in “isolated” form.

The term “gene” means the segment of DNA involved in producing a polypeptide chain; it includes regions preceding and following the coding region (“leader and trailer”) as well as intervening sequences (introns) between individual coding segments (exons).

A “functional variant” refers to a polypeptide or polynucleotide that is structurally similar or substantially structurally similar to a parent or reference compound of this disclosure, but differs, in some contexts slightly, in composition (e.g., one base, atom or functional group is different, added, or removed), such that the polypeptide or encoded polypeptide is capable of performing at least one function of the encoded parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Kₐ) or a dissociation (K_{D}) constant) or avidity; or an assay measuring IL-2 signaling (e.g.: measuring phosphorylated STAT5 by flow cytometry and/or western blotting using an antibody specific therefor (e.g. BD Phosflow™ Alexa Fluor® 647 Mouse Anti-Stat5 (pY694) from BD Biosciences, cat. no. 612599); measuring expression of one or more genes, the expression of which is known to be mediated by STAT5; measuring activation of a MAP kinase and/or of mTORC1); or an assay measuring TGFβ signaling (e.g., measuring phosphorylated SMAD2/SMAD3 by flow cytometry and/or western blotting using an antibody specific therefor (e.g. Human Phospho-Smad2 (S465/S467)/Smad3 (S423/S425) Antibody from R&D Systems, cat. no. AB3226; see also Phospho-Smad3 (S423/S425)/Smad2 (S465/S467) Cell-Based ELISA from R&D Systems, cat. no. KCB3226; see also Phospho-Smad2 (Ser465/467)/Smad3 (Ser423/425) (D27F4) Rabbit mAb #8828 from Cell Signaling Technology @). STAT5-mediated transcription can be interrogated using a known reporter assay such as the pGL3-3×STAT5-Luc reporter assay (Yin et al., J. Biomol Screen 16(4):443-449 (2011); doi: 10.1177/1087057111400190). Recruitment of GRB2 and SOS can be biochemically characterized, for example, by co-immunoprecipitation (Wang et al., J Biol. Chem. 275(30):23355-23361 (2000); doi: 10.1074/jbc.M000404200).

As used herein, a “functional portion” or “functional fragment” refers to a polypeptide or polynucleotide that comprises only a domain, portion or fragment of a parent or reference polypeptide or polynucleotide (respectively), and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., IL-2 signaling). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release).

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). It will be appreciated that in the case of a host cell that comprises a heterologous polynucleotide, the polynucleotide is “heterologous” to progeny of the host cell, whether or not the progeny were themselves manipulated to, for example, introduce the polynucleotide.

The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell (e.g., T cell) or microorganism targeted for genetic modification with a heterologous nucleic acid molecule to produce a polypeptide of interest (e.g., a fusion protein of the present disclosure). In certain embodiments, a host cell may optionally possess or be modified to include other genetic modifications that confer desired properties related or unrelated to, e.g., biosynthesis of the heterologous protein (e.g., inclusion of a detectable marker; deleted, altered or truncated endogenous host cell protein; expression of an antigen-binding protein).

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. Thus, depending upon the source of an original population of cells subjected to an enriching process, a mixture or composition may contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” cells. Cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, or 1% percent or less (in number or count) of the “depleted” cells. In certain embodiments, amounts of a certain cell type in a mixture will be enriched and amounts of a different cell type will be depleted, such as enriching for CD4⁺ cells while depleting CD8⁺ cells, or enriching for CD62L⁺ cells while depleting CD62L⁻ cells, or combinations thereof.

“T cell receptor” (TCR) refers to a multi-protein complex known as an immunoglobulin superfamily member (each component protein having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). The extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminus, and a constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. The variable domains contain complementary determining regions (CDRs) separated by framework regions (FRs) (see, e.g., Jores et al., Proc. Natl Acad. Sci. U.S.A. 87:9138, 1990; Chothia et al., EMBO J. 7:3745, 1988; see also Lefranc et al., Dev. Comp. Immunol. 27:55, 2003). The source of a TCR or TCR binding domain as used in the present disclosure may be from various animal species, such as a human, mouse, rat, rabbit or other mammal.

“CD3” is a multi-protein complex of six chains (see, Abbas and Lichtman, 2003; Janeway et al., p. 172 and 178, 1999). In mammals, the complex generally comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is thought to allow these chains to associate with positively charged regions of T cell receptor chains. The intracellular tails of the CD3 complex proteins contain immunoreceptor tyrosine-based activation motifs or ITAMs, which are thought to be important for T cell signaling in response to antigen binding.

CD3, as well as the protein subunits, domains, and sequences therefrom, may be from various animal species, including human, mouse, rat, or other mammals.

In certain embodiments, a TCR is found on the surface of T cells (also referred to as T lymphocytes) and associates with the CD3 complex. In certain embodiments, a TCR complex comprises a TCR or a functional portion thereof; a dimer comprising two CD3ζ chains, or functional portions or variants thereof; a dimer comprising a CD3δ chain and a CDε chain, or functional portions or variants thereof; and a dimer comprising a CD3γ chain and a CDε chain, or functional portions or variants thereof, any one or more of which may be endogenous or heterologous to the T cell.

“Major histocompatibility complex molecules” (MHC molecules) refer to glycoproteins that deliver peptide antigens to a cell surface. MHC class I molecules are heterodimers consisting of a membrane spanning a chain (with three a domains) and a non-covalently associated δ2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, cat, dog, goat, horse, or other mammals.

“CD4” refers to an immunoglobulin co-receptor glycoprotein that can assist the TCR in binding to antigen:MHC and communicating with antigen-presenting cells (see, Campbell & Reece, Biology 909 (Benjamin Cummings, Sixth Ed., 2002); UniProtKB P01730). CD4 is found on the surface of immune cells such as T helper cells, monocytes, macrophages, and dendritic cells, and includes four immunoglobulin domains (D1 to D4) that are expressed at the cell surface. During antigen recognition, CD4 is recruited, along with the TCR complex, to bind to different regions of the MHCII molecule (CD4 binds MHCII β2, while the TCR complex binds antigen:MHCII α1/β1).

As used herein, the term “CD8 co-receptor” or “CD8” means the cell surface glycoprotein CD8, either as an alpha-alpha homodimer or an alpha-beta heterodimer. The CD8 co-receptor can assist in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). In humans, there are five (5) different CD8 beta chains (see UniProtKB identifier P10966) and a single CD8 alpha chain (see UniProtKB identifier P01732).

“Chimeric antigen receptor” (CAR) refers to a fusion protein engineered to contain two or more amino acid sequences (which may be naturally occurring amino acid sequences) linked together in a way that does not occur naturally or does not occur naturally in a host cell, which fusion protein can function as an antigen-specific receptor when present on a surface of a cell. CARs of the present disclosure include an extracellular portion comprising an antigen-binding domain (e.g., obtained or derived from an immunoglobulin or immunoglobulin-like molecule, such as a scFv or scTCR derived from an antibody or TCR (respectively) specific for a cancer antigen, or an antigen-binding domain derived or obtained from a killer immunoreceptor from an NK cell, a designed ankyrin repeat protein (DARPin), an engineered fibronectin type three domain (also referred-to as a monobody) such as an Adnectin™, a ligand (e.g., a cytokine, if the target is a cytokine receptor), a receptor ectodomain (e.g., a cytokine receptor, if the target is a cytokine) or the like) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016); Stone et al., Cancer Immunol. Immunother., 63(11):1163 (2014)). In certain embodiments, a CAR comprises an antigen-specific TCR binding domain (see, e.g., Walseng et al., Scientific Reports 7:10713, 2017; the TCR CAR constructs and methods of which are hereby incorporated by reference in their entirety).

In the context of a TCR, the term “variable region” or “variable domain” refers to the domain of a TCR α-chain or β-chain (or γ-chain and δ-chain for γδ TCRs), or of an antibody heavy or light chain, that is involved in binding to antigen (i.e., contains amino acids and/or other structures that contact antigen and result in binding). The variable domains of the α-chain and β-chain (Vα and Vβ, respectively) of a native TCR generally have similar structures, with each domain comprising four generally conserved framework regions (FRs) and three CDRs. Variable domains of antibody heavy (V_{H}) and light (V_{L}) chains each also generally comprise four generally conserved framework regions (FRs) and three CDRs. In both TCRs and antibodies, framework regions separate CDRs and CDRs are situated between framework regions (i.e., in primary structure).

The terms “complementarity determining region,” and “CDR,” are synonymous with “hypervariable region” or “HVR,” and refer to sequences of amino acids within TCR or antibody variable regions, which, in general, confer antigen specificity and/or binding affinity and are separated from one another in primary structure by framework sequence. In some cases, framework amino acids can also contribute to binding, e.g., may also contact the antigen or antigen-containing molecule. In general, there are three CDRs in each variable region (i.e., three CDRs in each of the TCRα-chain and β-chain variable regions; 3 CDRs in each of the antibody heavy chain and light chain variable regions). In the case of TCRs, CDR3 is thought to be the main CDR responsible for recognizing processed antigen. CDR1 and CDR2 typically mainly interact with the MHC. Variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

“Antigen” or “Ag” as used herein refers to an immunogenic molecule that can provoke an immune response. This immune response may involve antibody production, activation of specific immunologically competent cells (e.g., T cells), secretion of cytokines, or any combination thereof. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

“Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a host cell expressing a fusion protein of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof. In some embodiments, a benefit of a cellular immunotherapy of this disclosure can further include a reduction (e.g., in number or severity) or absence of a cytokine-related toxicity, such as a cytokine release syndrome.

A “therapeutically effective amount” or “effective amount” of a composition (fusion protein, host cell expressing a fusion protein, polynucleotide, vector, or the like) of this disclosure, refers to an amount of the composition sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. In the case of cancers, benefits can include, for example, a reduction in the size, area, volume, and/or density of a tumor, and/or a reduction or reversal in the rate of tumor growth or spread of cancer,

When referring to an individual active ingredient, administered alone, a therapeutically effective amount refers to the effects of that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient, such as two different antigen-binding proteins (e.g., CARs, TCRs) that specifically bind an antigen, or a fusion protein of the present disclosure.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

As used herein, “statistically significant” refers to a p-value of 0.050 or less when calculated using the Student's t-test and indicates that it is unlikely that a particular event or result being measured has arisen by chance.

As used herein, the term “adoptive immune therapy” or “adoptive immunotherapy” refers to administration of naturally occurring or genetically engineered, disease-antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

As used herein, the term “optimally aligned” in the context of two or more nucleic acids or polypeptide sequences, refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

Included in the current disclosure are variants of any of the fusion proteins or components or domains thereof described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic function(s) of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to any one of the fusion protein or component sequences described herein. In some embodiments, such conservatively substituted variants are functional variants. Such functional variants can encompass sequences with substitutions such that the activity of one or more critical active site residues or ligand binding residues is conserved.

### Fusion Proteins

In some embodiments, a fusion protein is provided that comprises: (i) an extracellular component comprising an extracellular domain of a transforming growth factor beta receptor (TGFβR) polypeptide, or a portion or variant thereof that is capable of binding to a TGFβ polypeptide; (ii) an intracellular component comprising an intracellular portion of an interleukin-2 receptor (IL-2R) polypeptide; and (iii) a transmembrane component disposed between the extracellular component of (i) and the intracellular component of (ii), wherein the fusion protein, when expressed by a host cell and bound to the TGFβ polypeptide, is capable of contributing to an IL-2 signal in the host cell.

In certain embodiments, the TGFβR polypeptide comprises a TGFβRI polypeptide or a TGFβR2 polypeptide. In certain embodiments, the IL-2R polypeptide comprises an IL-2Rβ polypeptide, an IL-2Rγ polypeptide, or both.

In some embodiments, a fusion protein is provided that comprises: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor beta (IL-2Rβ) polypeptide; and (iii) a transmembrane component disposed between the extracellular component of (i) with the intracellular component of (ii), wherein the fusion protein, when expressed by a host cell and bound to the TGFβ polypeptide, is capable of contributing to an IL-2 signal in the host cell.

In some embodiments, the extracellular component comprising an extracellular domain of a TGFβR1 or TGFβR2 polypeptide or a functional fragment thereof comprises a human or rat TGFβR1 or TGFβR2 polypeptide (e.g. uniprot accession numbers P36897, A3QNQ0, P80204, P38438, which entries, sequence information, and protein characteristics are incorporated in their entireties herein).

In some embodiments, the extracellular component comprising an extracellular domain of a TGFβR1 or TGFβR2 polypeptide, a functional fragment thereof, or a variant thereof is configured to bind one or more residues of the natural ligands for TGFβR1 or TGFβR2, including but not limited to at least one of TGF-β1, TGF-β2, TGF-β3, BMP-2, BMP-7, or ActA. In some embodiments, the one or more residues of the natural ligands comprise residues identified as important for the TGFβR1- or TGFβR2-ligand interaction (see e.g. Hart et al. Nat Struct Biol. 2002 March; 9(3):203-8, which is incorporated by reference in its entirety herein, including the crystal structures and binding interaction teachings therein). In some embodiments, the extracellular component is configured to contact at least one of residues R25, K31, W32, H34, K37, Y90, Y91, V92, G93, or R94 of TGF-β3, TGF-β2, or TGF-β1. In some embodiments, the extracellular component is configured to contact at least one of residues R25, K31, W32, H34, K37, Y90, Y91, V92, G93, or R94 relative to TGF-β3, TGF-β2, or TGF-β1 when optimally aligned. In some embodiments, the extracellular component comprises at least one of residues L27, F30, D32, S49, I50, T51, S52, 153, or E55 of human TGF-βR2. In some embodiments, the extracellular component comprises at least one of residues L27, F30, D32, S49, I50, T51, S52, 153, or E55 relative to human TGF-βR2 when optimally aligned.

In some embodiments, a fusion protein intracellular component that comprises an intracellular portion of an IL-2Rβ or IL-2Rγ polypeptide comprises one or more residues characteristic of SH2 signalling domains, one or more residues conserved between human and murine IL-2Rβ or IL-2Rγ, and/or one or more residues capable of phosphorylation (see e.g. Nelson et al. Mol Cell Biol. 1996 January; 16(1):309-17, which is incorporated by reference herein in its entirety). In some embodiments, the one or more residues comprise P4, R5, I6, P7, T8, L12, D14, L15, V16, Y19, G32, L33, E35, or L37 of the human IL-2Rγ intracellular domain. In some embodiments, the one or more residues comprise P4, R5, I6, P7, T8, L12, D14, L15, V16, Y19, G32, L33, E35, or L37 relative to SEQ ID NO: 46 when optimally aligned

In some embodiments, the intracellular component of a fusion protein comprises, consists essentially of, or consists of a membrane-proximal region of IL-2Rγ (e.g., comprising cytoplasmic residues 1-37 or 1-52 or 5-37 or 40-52; see Nelson et al., Mol. Cell Bio. 16(1):309-317 (1996)).

In some embodiments, a fusion protein is provided that comprises: (i) an extracellular component comprising an extracellular domain of a transforming growth factor beta-receptor 2 (TGFβR2) polypeptide, or a portion or variant thereof that is capable of binding to a TGFβ polypeptide; (ii) an intracellular component comprising an intracellular portion of an interleukin-2 receptor beta (IL-2Rβ) polypeptide; and (iii) a transmembrane component disposed between the extracellular component of (i) with the intracellular component of (ii), wherein the fusion protein, when expressed by a host cell and bound to the TGFβ polypeptide, is capable of contributing to an IL-2 signal in the host cell.

In some embodiments, a fusion protein is provided that comprises: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor gamma (IL-2Rγ) polypeptide; and (iii) a transmembrane component disposed between the extracellular component of (i) with the intracellular component of (ii), wherein the fusion protein, when expressed by a host cell and bound to the TGFβ polypeptide, is capable of contributing to an IL-2 signal in the host cell.

In some embodiments, a fusion protein is provided that comprises: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor gamma (IL-2Rγ) polypeptide; and (iii) a transmembrane component disposed between the extracellular component of (i) with the intracellular component of (ii), wherein the fusion protein, when expressed by a host cell and bound to the TGFβ polypeptide, is capable of contributing to an IL-2 signal in the host cell.

In any of the presently disclosed embodiments, the extracellular component can comprise or consist of an extracellular domain from TGFβRI (e.g. SEQ ID NO.:39) or from TGFβR2 (e.g. SEQ ID NO.:41).

In any of the presently disclosed embodiments, the transmembrane component can comprise or consist of a transmembrane domain from IL-2Rβ (SEQ ID NO.:43).

In any of the presently disclosed embodiments, the transmembrane component can comprise or consist of a transmembrane domain from IL-2Rγ (SEQ ID NO.:45).

In any of the presently disclosed embodiments, the transmembrane component can comprise or consist of a transmembrane domain from TGFβR1 (SEQ ID NO.:40). In some embodiments, a fusion protein comprises a transmembrane domain from TGFβR1 and, extending from the transmembrane domain into the intracellular component of the fusion protein, an intracellular overhang or overhang sequence from TGFβR1, as described for Table A. In some embodiments, the intracellular overhang sequence comprises, consists essentially of, or consists of C-H-N.

In any of the presently disclosed embodiments, the transmembrane component can comprise or consist of a transmembrane domain from TGFβR2 (SEQ ID NO.:42). In some embodiments, a fusion protein comprises a transmembrane domain from TGFβR2 and, extending from the transmembrane domain into the intracellular component of the fusion protein, an intracellular overhang or overhang sequence from TGFβR2, as described for Table A. In some embodiments, the intracellular overhang sequence comprises, consists essentially of, or consists of R-V-N.

In any of the presently disclosed embodiments, the intracellular component can comprise an intracellular domain from IL-2Rγ (SEQ ID NO.:46) or from IL-2Rβ (SEQ ID NO.:44).

In some embodiments, a fusion protein is provided that comprises: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor beta (IL-2Rβ) polypeptide, wherein the intracellular component optionally comprises an IL-2Rβ intracellular domain; and (iii) a transmembrane component disposed between the extracellular component of (i) with the intracellular component of (ii), wherein the transmembrane component optionally comprises an IL-2Rβ transmembrane domain, wherein the fusion protein, when expressed by a host cell and bound to the TGFβ polypeptide, is capable of contributing to an IL-2 signal in the host cell.

In some embodiments, a fusion protein is provided that comprises: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor beta (IL-2Rβ) polypeptide, wherein the intracellular component optionally comprises an IL-2Rβ intracellular domain; and (iii) a transmembrane component disposed between the extracellular component of (i) with the intracellular component of (ii), wherein the transmembrane component optionally comprises an IL-2Rβ transmembrane domain, wherein the fusion protein, when expressed by a host cell and bound to the TGFβ polypeptide, is capable of contributing to an IL-2 signal in the host cell.

In some embodiments, a fusion protein is provided that comprises: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor beta (IL-2Rβ) polypeptide, wherein the intracellular component optionally comprises a IL-2Rβ transmembrane domain; and (iii) a transmembrane component disposed between the extracellular component of (i) with the intracellular component of (ii), wherein the transmembrane component optionally comprises a TGFβR1 transmembrane domain (in which case the intracellular component can further comprise a TGFβR1 intracellular overhang or overhang sequence), wherein the fusion protein, when expressed by a host cell and bound to the TGFβ polypeptide, is capable of contributing to an IL-2 signal in the host cell.

In some embodiments, a fusion protein is provided that comprises: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor beta (IL-2Rβ) polypeptide, wherein the intracellular component optionally comprises a IL-2Rβ intracellular domain; and (iii) a transmembrane component disposed between the extracellular component of (i) with the intracellular component of (ii), wherein the transmembrane component optionally comprises a TGFβR2 transmembrane domain (in which case the intracellular component can further comprise a TGFβR2 intracellular overhang or overhang sequence), wherein the fusion protein, when expressed by a host cell and bound to the TGFβ polypeptide, is capable of contributing to an IL-2 signal in the host cell.

In some embodiments, a fusion protein is provided that comprises: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor gamma (IL-2Rγ) polypeptide, wherein the intracellular component optionally comprises a IL-2Rγ intracellular domain; and (iii) a transmembrane component disposed between the extracellular component of (i) with the intracellular component of (ii), wherein the transmembrane component optionally comprises an IL-2Rγ transmembrane domain, wherein the fusion protein, when expressed by a host cell and bound to the TGFβ polypeptide, is capable of contributing to an IL-2 signal in the host cell.

In some embodiments, a fusion protein is provided that comprises: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor gamma (IL-2Rγ) polypeptide, wherein the intracellular component optionally comprises a IL-2Rγ intracellular domain; and (iii) a transmembrane component disposed between the extracellular component of (i) with the intracellular component of (ii), wherein the transmembrane component optionally comprises an IL-2Rγ transmembrane domain, wherein the fusion protein, when expressed by a host cell and bound to the TGFβ polypeptide, is capable of contributing to an IL-2 signal in the host cell.

In some embodiments, a fusion protein is provided that comprises: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor gamma (IL-2Rγ) polypeptide, wherein the intracellular component optionally comprises a IL-2Rγ intracellular domain; and (iii) a transmembrane component disposed between the extracellular component of (i) with the intracellular component of (ii), wherein the transmembrane component optionally comprises an TGFβR2 transmembrane domain (in which case the intracellular component can further comprise a TGFβR2 intracellular overhang or overhang sequence), wherein the fusion protein, when expressed by a host cell and bound to the TGFβ polypeptide, is capable of contributing to an IL-2 signal in the host cell.

In some embodiments, a fusion protein is provided that comprises: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor gamma (IL-2Rγ) polypeptide, wherein the intracellular component optionally comprises a IL-2Rγ intracellular domain; and (iii) a transmembrane component disposed between the extracellular component of (i) with the intracellular component of (ii), wherein the transmembrane component optionally comprises an TGFβRI transmembrane domain (in which case the intracellular component can further comprise a TGFβRI intracellular overhang or overhang sequence), wherein the fusion protein, when expressed by a host cell and bound to the TGFβ polypeptide, is capable of contributing to an IL-2 signal in the host cell.

In certain embodiments of a fusion protein, (1)(i) the extracellular component has at least 90%, at least 91%, at least 92%, at least 93%, at least 94%, at least 95%, at least 96%, at least 97%, at least 98%, or at least 99% identity to, or comprises or consists of, the amino acid sequence set forth in SEQ ID NO.:39 or 41; (ii) the transmembrane component has at least 90%, at least 91%, at least 92%, at least 93%, at least 94%, at least 95%, at least 96%, at least 97%, at least 98%, or at least 99% identity to, or comprises or consists of, the amino acid sequence set forth in: SEQ ID NO.:40; SEQ ID NO.:42; SEQ ID NO.:43; or SEQ ID NO.:45; and (iii) the intracellular component has at least 90%, at least 91%, at least 92%, at least 93%, at least 94%, at least 95%, at least 96%, at least 97%, at least 98%, or at least 99% identity to, or comprises or consists of, the amino acid sequence set forth in SEQ ID NO.:44 or 46, and wherein, optionally, the fusion protein comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOS.:5, 6, 9, 10, 13, 14, 17, and 18; and/or (2) wherein the fusion protein comprises one or more amino acid substitutions, insertions, and/or deletions to reduce or prevent the fusion protein from forming a protein dimer with a human TGFβR1 or a human TGFβR2, wherein, optionally, the one or more amino acid substitutions, insertions, and/or deletions provide: (i) a variant of SEQ ID NO.:47, wherein the variant comprises from one to ten amino acid insertions, or from one to ten amino acid deletions; (ii) one, two, three, four, five, six, seven, eight, nine, ten, or more amino acids added to the N-terminus and/or to the C-terminus of SEQ ID NO.:47; (iii) a variant of SEQ ID NO.:48, wherein the variant comprises from one to ten amino acid insertions, or from one to ten amino acid deletions; (iv) one, two, three, four, five, six, seven, eight, nine, ten, or more added to the N-terminus and/or to the C-terminus of SEQ ID NO.:48.

In any of the presently disclosed embodiments, the IL-2 signal comprises any one or more of (i)-(vii): (i) phosphorylation of the intracellular portion of the IL-2R polypeptide by JAK1 or JAK3; (ii) phosphorylation in the host cell of any one or more of: PI3K; Akt; STAT; STAT5A; STAT5B; MEK; SHC1; MEK1; MEK2; ERK1; ERK2; and STAT3 (e.g. using an antibody specific for the phosphorylated form of the protein); (iii) STAT5-mediated transcription; (iv) recruitment of GRB2 and SOS; (v) GTP loading of RAS; (vi) activation of the Raf-ERK MAPK cascade; (vii) activation of mTORC1 and/or of HIF1α/HIF1β.

In any of the presently disclosed embodiments, when the fusion protein is expressed by a host cell (e.g. in some embodiments, the host cell expresses two fusion proteins as described herein and the fusion proteins are comprised in a TGFβ-binding complex) and the fusion protein (or complex) binds to the TGFβ polypeptide, the host cell performs one or more of the following: proliferation; growth; expression of an effector molecule (e.g., a T cell receptor); glycolytic metabolism; expression of protein (e.g., MYC, SLC7A5, or both); and biosynthesis of cholesterol.

In some embodiments, a fusion protein is provided that comprises or consists of the amino acid sequence set forth in any one of SEQ ID NOs.:5, 6, 9, 10, 13, 14, 17, and 18.

In certain embodiments, a fusion protein is provided that comprises or consists of the amino acid sequence set forth in any one of SEQ ID NOs.:7, 11, 15, and 19.

### Antigen-Binding Proteins

In some embodiments, a polynucleotide encoding a fusion protein as provided herein, or a host cell comprising or expressing the same, further encodes or expresses an antigen-binding protein. An antigen-binding protein will comprise at least an extracellular binding domain, and will typically comprise a transmembrane component and an intracellular component. In certain embodiments, the intracellular component can comprise a signaling domain, a costimulatory domain, or both. Examples of antigen-binding proteins include TCRs, CARs, scTCRs, and the like.

A “binding domain” (also referred to as a “binding region” or “binding moiety”), as used herein, refers to a molecule or portion thereof (e.g., peptide, oligopeptide, polypeptide) that possesses the ability to specifically and non-covalently associate, unite, or combine with a target (e.g. antigen). A binding domain includes any naturally occurring, synthetic, semi-synthetic, or recombinantly produced binding partner for a biological molecule, a molecular complex (i.e., complex comprising two or more biological molecules), or other target of interest. Exemplary binding domains useful in the fusion proteins include single chain immunoglobulin variable regions (e.g., scTCR, scFv, scFab, scTv), Fabs, sdAbs such as nanobodies/VHH, VNAR, receptor ectodomains, ligands (e.g., cytokines, chemokines), or (other) synthetic polypeptides selected for their specific ability to bind to a biological molecule, a molecular complex or other target of interest (e.g., DARPins, ¹⁰FNIII domains). In certain embodiments, the binding domain comprises a scFv, scTv, scTCR, or ligand. In certain embodiments, the binding domain is chimeric, human, or humanized.

In certain embodiments, the binding domain is or comprises a scFv comprising a V_{H} domain, a V_{L} domain, and a peptide linker. In particular embodiments, a scFv comprises a V_{H} domain joined to a V_{L} domain by a peptide linker, which can be in a V_{H}-linker-V_{L} orientation or in a V_{L}-linker-V_{H} orientation.

An scFv may be engineered so that the C-terminal end of the V_{L} domain is linked by a short peptide sequence to the N-terminal end of the V_{H} domain, or vice versa (i.e., (N)V_{L}(C)-linker-(N)V_{H}(C) or (N)V_{H}(C)-linker-(N)V_{L}(C). It will be appreciated that a scTCR or a scTv or a scFab may also be designed in any N-terminal to C-terminal orientation.

As used herein, “specifically binds” or “specific for” refers to an association or union of an antigen-binding protein (e.g., a T cell receptor or a chimeric antigen receptor) or a binding domain (or fusion protein comprising the same) to a target molecule with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Antigen-binding proteins or binding domains may be classified as “high-affinity” binding proteins or binding domains or as “low-affinity” binding proteins or binding domains. “High-affinity” binding proteins or binding domains refer to those binding proteins or binding domains having a Kₐ of at least 10⁷ M⁻¹, at least 10⁸ M⁻¹, at least 10⁹ M¹, at least 10¹⁰ M⁻¹, at least 10¹¹ M⁻¹, at least 10¹² M⁻¹, or at least 10¹³ M⁻¹. “Low-affinity” binding proteins or binding domains refer to those binding proteins or binding domains having a Kₐ of up to 10⁷ M⁻¹, up to 10⁶ M⁻¹, or up to 10⁵ M⁻¹. Alternatively, affinity may be defined as an equilibrium dissociation constant (K_{D}) of a particular binding interaction with units of M (e.g., 10⁻⁵ M to 10⁻¹³ M).

In further embodiments, an antigen-binding protein can specifically bind to a target antigen (e.g., a cancer antigen such as, for example, a ROR1, CD19, CD20, CD22, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, MAGE-A3, MAGE-A4, SSX-2, PRAME, HA-1, Core Binding Factor (CBF), PSA, ephrin A2, ephrin B2, an NKG2D, NY-ESO-1, TAG-72, mesothelin, NY-ESO, α-fetoprotein, CAR15-3, hCG or beta-hcG, 5T4, BCMA, FAP, Carbonic anhydrase 9, BRAF (such as BRAF^{V600E}), p2M, ETA, tyrosinase, KRAS, NRAS, MR1, or CEA antigen), optionally comprised in an antigen:MHC complex. In some embodiments, an antigen-binding protein or binding domain thereof is capable of specifically binding to an autoimmune antigen, or an antigen that is associated with an infection (e.g., viral, bacterial, fungal, or parasitic), or an antigen that is associated with a neurodegenerative disease (e.g., tau, alpha-synuclein, amyloid beta).

In certain embodiments, a binding domain of an antigen-binding protein may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof.

A variety of assays are known for identifying binding domains that specifically bind a particular target, as well as determining binding domain or antigen-binding protein affinities, such as Western blot, ELISA, analytical ultracentrifugation, spectroscopy, isothermal titration calorimetry (ITC), and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for apparent affinity or relative affinity are also known. In certain examples, apparent affinity for an antigen-binding protein is measured by assessing binding to various concentrations of tetramers, for example, by flow cytometry using labeled tetramers. In some examples, apparent K_{D} of a fusion protein is measured using 2-fold dilutions of labeled tetramers at a range of concentrations, followed by determination of binding curves by non-linear regression, apparent K_{D} being determined as the concentration of ligand that yielded half-maximal binding.

In certain embodiments, in addition to the binding domain, the extracellular component of an antigen-binding protein comprises. (i) an immunoglobulin (e.g., IgG, such as IgG1, IgG2, IgG3, or IgG4) CHI domain, or a functional variant or portion thereof; (ii) an immunoglobulin (e.g., IgG, such as IgG1, IgG2, IgG3, or IgG4) CH2 domain, or a functional variant or portion thereof; (iii) an immunoglobulin (e.g., IgG, such as IgG1, IgG2, IgG3, or IgG4) CH3 domain, or a functional variant or portion thereof; (iv) an immunoglobulin (e.g., IgG, such as IgG1, IgG2, IgG3, or IgG4) CL domain, or a functional variant or portion thereof; (v) a CD8 extracellular domain, or a functional variant or portion thereof; (vi) a CD28 extracellular domain, or a functional variant or portion thereof; (vii) a CD4 extracellular domain, or a functional variant or portion thereof (viii) an IgG (e.g., IgG1, IgG2, IgG3, or IgG4) hinge (e.g., comprising or consisting of the amino acid sequence of SEQ ID NO:71), or a functional variant or portion thereof; (ix) a type II C-lectin interdomain (stalk) region, or a functional variant or portion thereof, (x) a cluster of differentiation (CD) molecule stalk region or a functional variant thereof, (xi) a linker, optionally a glycine-serine linker comprising from about one to about ten repeats of GlyₓSer_{y}, wherein X and Y are each independently from one to ten; or (xii) any combination of (i)-(xi). In general, the one or more of (i)-(xii) will be disposed between the transmembrane domain and the binding domain. It will be appreciated that a functional variant or portion thereof of a CH1 domain, CH2 domain, CH3 domain, CL domain, CD8 extracellular domain, CD28 extracellular domain, CD4 extracellular domain, type II C-lectin interdomain (stalk) region, cluster of differentiation (CD) molecule stalk region, or IgG hinge (e.g., linkers, as discussed further below) in the context of a antigen-binding protein, is of sufficient length, shape, and/or flexibility to position the binding domain away from the surface of a host cell expressing the antigen-binding protein to enable proper contact between the host cell and a target cell, target (e.g., antigen) binding, and activation of the host cell. In certain embodiments, the extracellular domain comprises a linker disposed between (and optionally, but not necessarily, connecting) the binding domain and the transmembrane domain. In some embodiments, the linker comprises a hinge region or a portion thereof, optionally an IgG hinge amino acid sequence.

An extracellular component and an intracellular component of an antigen-binding protein of the present disclosure are connected by a transmembrane domain. In at least the context of an antigen-binding protein, a “transmembrane domain” is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In certain embodiments, the transmembrane domain of an antigen-binding protein comprises or is derived from a known transmembrane protein (e.g., a CD4 transmembrane domain, a CD8 transmembrane domain, a CD27 transmembrane domain, a CD28 transmembrane domain, or any combination thereof), and can be a functional portion or variant thereof; i.e., that retains or substantially retains a three-dimensional structure that is thermodynamically stable in a cell membrane and generally having a length from about 15 amino acids to about 30 amino acids.

In certain embodiments, the extracellular component of the antigen-binding protein further comprises a linker disposed between (and optionally, but not necessarily, connecting) the binding domain and the transmembrane domain. As used herein when referring to a component of a antigen-binding protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker of the present disclosure can position the binding domain away from the surface of a host cell expressing the antigen-binding protein to enable proper contact between the host cell and a target cell, target (e.g., antigen) binding, and activation of the host cell (Patel et al., Gene Therapy 6: 412-419, 1999). Linker length may be varied to maximize antigen recognition based on the selected target molecule, selected binding epitope, or antigen binding domain size and affinity (see, e.g., Guest et al., J. Immunother. 28:203-11, 2005; PCT Publication No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂; (Gly₃Ser)₂; Gly₂Ser; or a combination thereof, such as (Gly₃Ser)₂Gly₂Ser). In some embodiments, the extracellular domain comprises a glycine-serine linker that is not comprised in the binding domain; e.g., is disposed between the transmembrane domain and the binding domain (irrespective of whether the binding domain also comprises such a linker). For example, in certain embodiments, an antigen-binding protein comprises an extracellular domain comprising a first glycine-serine linker disposed between the transmembrane domain and the binding domain, and the binding domain may comprise a scFv or an scTCR or an scTv or an scFab that comprises a second glycine-serine linker, wherein the first and second glycine-serine linkers may be a same or a different glycine-serine linker and may be of a same or a different length.

Linkers for use in antigen-binding proteins also include immunoglobulin constant regions (i.e., CH1, CH2, CH3, or CL, of any isotype) and portions and variants thereof. In certain embodiments, the linker comprises a CH3 domain, a CH2 domain, or both. In certain embodiments, the linker comprises a CH2 domain and a CH3 domain. In further embodiments, the CH2 domain and the CH3 domain are each a same isotype. In particular embodiments, the CH2 domain and the CH3 domain are an IgG4 or IgG1 isotype. In other embodiments, the CH2 domain and the CH3 domain are each a different isotype. In specific embodiments, the CH2 comprises a N297Q mutation. Without wishing to be bound by theory, it is believed that CH2 domains with N297Q mutation do not bind FcγR (see, e.g., Sazinsky et al., PNAS 105(51):20167 (2008)). In certain embodiments, the linker comprises a human immunoglobulin constant region or a portion thereof. In certain embodiments, the linker comprises an extracellular domain from CD4, or a portion thereof. In some embodiments, the linker comprises an extracellular domain from CD8, or a portion thereof.

In any of the embodiments described herein, a linker may comprise a hinge region or a portion thereof. Hinge regions are flexible amino acid polymers of variable length and sequence (typically rich in proline and cysteine amino acids) and connect larger and less-flexible regions of immunoglobulin proteins. For example, hinge regions connect the Fc and Fab regions of antibodies and connect the constant and transmembrane regions of TCRs. In certain embodiments, the linker comprises an immunoglobulin constant region or a portion thereof and a hinge region or a portion thereof. In certain embodiments, the linker comprises a glycine-serine linker as described herein.

In certain embodiments, the intracellular component of the antigen-binding protein comprises an effector domain, or a functional portion or variant thereof.

In at least the context of an antigen-binding protein, an “effector domain” is an intracellular portion or domain of an antigen-binding protein that can directly or indirectly promote a biological or physiological response in a cell when receiving an appropriate signal. In certain embodiments, a biological or physiological response is or comprises an immune response. In certain embodiments, an effector domain is from a protein or portion thereof or protein complex that receives a signal when bound, or when the protein or portion thereof or protein complex binds directly to a target molecule and triggers a signal from the effector domain.

An effector domain may directly promote a cellular response when it contains one or more signaling domains or motifs, such as an Intracellular Tyrosine-based Activation Motif (ITAM), such as those found in costimulatory molecules. Without wishing to be bound by theory, it is believed that ITAMs are important for T cell activation following ligand engagement by a T cell receptor or by a fusion protein comprising a T cell effector domain. In certain embodiments, the intracellular component or functional portion thereof comprises an ITAM.

Exemplary effector domains that may be included in an antigen-binding protein of the present disclosure include those from CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or a functional portion or variant thereof, or any combination thereof.

In certain embodiments, the intracellular component further comprises a costimulatory domain or a functional portion or variant thereof, wherein the costimulatory domain or functional portion or variant thereof is optionally disposed between the effector domain and the transmembrane domain.

In certain embodiments, the intracellular component of the antigen-binding protein comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or a functional variant thereof, or any combination thereof. In certain embodiments, the intracellular component comprises a CD28 costimulatory domain or a functional portion or variant thereof (which may optionally include a LL→GG mutation at positions 186-187 of the native CD28 protein (see Nguyen et al., Blood 102:4320, 2003)), a 4-1BB costimulatory domain or a functional portion or variant thereof, or both.

In certain embodiments, an intracellular component of an antigen-binding protein comprises a CD3ζ endodomain or a functional (e.g., signaling) portion thereof, or a functional variant thereof. In certain embodiments, an intracellular component of an antigen-binding protein comprises a CD27 endodomain or a functional (e.g., signaling) portion thereof, or a functional variant thereof. In certain embodiments, an intracellular component of an antigen-binding fusion protein comprises a CD28 endodomain or a functional (e.g., signaling) portion thereof, or a functional variant thereof. In still further embodiments, an intracellular component of an antigen-binding protein comprises a 4-1BB endodomain or a functional (e.g., signaling) portion thereof, or a functional variant thereof. In certain embodiments, an intracellular component of an antigen-binding protein comprises an OX40 endodomain or a functional (e.g., signaling) portion thereof, or a functional variant thereof. In certain embodiments, an intracellular component of an antigen-binding protein comprises a CD2 endodomain or a functional (e.g., signaling) portion thereof, or a functional variant thereof. In certain embodiments, an intracellular component of an antigen-binding protein comprises a CD5 endodomain or a functional (e.g., signaling) portion thereof, or a functional variant thereof. In certain embodiments, an intracellular component of an antigen-binding protein comprises an ICAM-1 endodomain or a functional (e.g., signaling) portion thereof, or a functional variant thereof. In certain embodiments, an intracellular component of an antigen-binding protein comprises a LFA-1 endodomain or a functional (e.g., signaling) portion thereof, or a functional variant thereof. In certain embodiments, an intracellular component of an antigen-binding protein comprises an ICOS endodomain or a functional (e.g., signaling) portion thereof, or a functional variant thereof.

In certain embodiments, one or more of an extracellular component, a binding domain, a linker, a transmembrane domain, an intracellular component, an effector domain or functional portion or variant thereof, or a costimulatory domain or functional portion or variant thereof, of an antigen-binding protein can (or a fusion protein can) further comprise one or more junction amino acids. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a fusion protein (e.g., amino acid residues resulting from the use of a restriction enzyme site or self-cleaving peptide sequences during the construction of a polynucleotide encoding a fusion protein). For example, a transmembrane domain of a fusion protein may have one or more junction amino acids at the amino-terminal end, carboxy-terminal end, or both.

Protein tags are unique peptide sequences that are affixed or genetically fused to, or are a part of, a protein of interest and can be recognized or bound by, for example, a heterologous or non-endogenous cognate binding molecule or a substrate (e.g., receptor, ligand, antibody, carbohydrate, or metal matrix) or a fusion protein of this disclosure. Protein tags can be useful for detecting, identifying, isolating, tracking, purifying, enriching for, targeting, or biologically or chemically modifying tagged proteins of interest, particularly when a tagged protein is part of a heterogeneous population of cell proteins or cells (e.g., a biological sample like peripheral blood). In certain embodiments, a protein tag of a fusion protein or antigen-binding protein of this disclosure comprises a Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, Strep tags (e.g., Strep-Tag; Strep-Tag II; and variants thereof, including those disclosed in, for example, Schmidt and Skerra, Nature Protocols, 2:1528-1535 (2007), U.S. Pat. No. 7,981,632; and PCT Publication No. WO 2015/067768, the strep-tag peptides, step-tag-peptide-containing polypeptides, and sequences of the same, are incorporated herein by reference), or any combination thereof.

In any of the embodiments described herein, an antigen-binding protein can be or can comprise a CAR or a TCR. Methods for making fusion proteins, including CARs, are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; Brentjens et al., 2007, Clin. Cancer Res. 13:5426, and Walseng et al., Scientific Reports 7:10713, 2017, the techniques of which are herein incorporated by reference. Methods for producing engineered TCRs are described in, for example, Bowerman et al., Mol. Immunol., 46(15):3000 (2009), the techniques of which are herein incorporated by reference.

In certain embodiments, the TCR comprises a single chain TCR (scTCR), which comprises both TCR variable domains (e.g. Vα and Vβ domains), but only a single TCR constant domain (e.g. Cα or Cβ). In certain embodiments, the antigen-binding fragment of the TCR, or chimeric antigen receptor, is chimeric (e.g., comprises amino acid residues or motifs from more than one donor or species), humanized (e.g., comprises alterations in amino acid sequence from a source non-human protein so as to reduce the risk of immunogenicity in a human), or human.

Methods useful for isolating and purifying recombinantly produced soluble fusion proteins and/or antigen-binding proteins, by way of example, may include obtaining supernatants from suitable host cell/vector systems that secrete the recombinant soluble fusion protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble fusion protein described herein include batch cell culture, which is monitored and controlled to maintain appropriate culture conditions. Purification of the soluble fusion protein may be performed according to methods described herein and known in the art and that comport with laws and guidelines of domestic and foreign regulatory agencies.

Antigen-binding proteins as described herein may be functionally characterized according to any of a large number of art-accepted methodologies for assaying host cell activity. For example, in the case of a host T cell, antigen-binding proteins can be functionally characterized by determination of T cell binding, activation or induction, as well as determination of T cell responses that are target (e.g., antigen)-specific. Examples include determination of T cell proliferation, T cell cytokine release, target-specific T cell stimulation, MHC-restricted T cell stimulation, CTL activity (e.g., by detecting ⁵¹Cr or Europium release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, MA (1986); Mishell and Shigii (eds.) Selected Methods in Cellular Immunology, Freeman Publishing, San Francisco, CA (1979); Green and Reed, Science 281:1309 (1998) and references cited therein. Certain of these assays may be used to assess activity of a TGFβ-binding fusion protein of the present disclosure, or of a host cell comprising the same.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an target-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

Polynucleotides. Vectors, and Host Cells

In certain aspects, nucleic acid molecules (also referred-to as polynucleotides) are provided that encode any one or more of the fusion proteins as described herein, and optionally an antigen-binding protein. A polynucleotide encoding a desired fusion protein of this disclosure can be inserted into an appropriate vector (e.g., viral vector or non-viral plasmid vector) for introduction into a host cell of interest (e.g., an immune cell, such as a T cell). A polynucleotide can further encode additional fusion proteins as disclosed herein, and/or can further encode an antigen-binding protein, and/or can further encode a marker.

In some embodiments, a polynucleotide encodes two fusion proteins, and a sequence encoding a fusion protein comprising a TGFβRII extracellular domain or functional portion or variant thereof is disposed 5′ to a sequence encoding a fusion protein comprising a TGFβRI extracellular domain or functional portion or variant thereof, and optionally 5′ to any other polypeptide encoded by the polynucleotide. Without wishing to be bound by theory, placing a cistron encoding a TGFβRII-derived fusion protein in the first (5′-most) position in a multicistronic construct may facilitate the highest expression of this cistron out of all cistrons in the construct; increased surface expression of a TGFβRII-derived fusion protein may provide improved sensitivity to TGFβ binding and/or improved IL-2R signal transduction due to the TGFβRII having a higher affinity (as compared to TGFβRI for TGFβ).

Exemplary markers (e.g., for transduction of a cell with a polynucleotide as provided herein) include green fluorescent protein, an extracellular domain of human CD2, a truncated human EGFR (huEGFRt, (see Wang et al., Blood 118:1255, 2011), a truncated human CD19 (huCD19t); a truncated human CD34 (huCD34t); or a truncated human NGFR (huNGFRt). In certain embodiments, an encoded marker comprises EGFRt, CD19t, CD34t, or NGFRt.

In any of presently disclosed embodiments, a (fusion or antigen-binding) protein-encoding polynucleotide can further comprise a polynucleotide that encodes a marker and a polynucleotide that encodes a self-cleaving polypeptide, wherein the polynucleotide encoding the self-cleaving polypeptide is located between the polynucleotide encoding the (fusion or antigen-binding) protein and the polynucleotide encoding the marker (or between polynucleotides encoding different fusion proteins, or between polynucleotides encoding components of an antigen-binding protein (e.g. TCR chains), or between a polynucleotide encoding a fusion protein and a polynucleotide encoding an antigen-binding protein or a component thereof). For example, when the protein-encoding polynucleotide, marker-encoding polynucleotide, and self-cleaving polypeptide are expressed by a host cell, the (fusion or antigen-binding) protein and the marker will be present on the host cell surface as separate molecules. In certain embodiments, a self-cleaving polypeptide comprises a 2A peptide from porcine teschovirus-1 (P2A, Thoseaasigna virus (T2A, equine rhinitis A virus (E2A), foot-and-mouth disease virus (F2A), Theilovirus, and encephalomocarditis virus). Exemplary nucleic acid and amino acid sequences of 2A peptides are set forth in, for example, Kim et al. (PLOS One 6:e18556, 2011, which 2A nucleic acid and amino acid sequences are incorporated herein by reference in their entirety; also incorporated by reference herein are the 2A and 2A-like peptides described in Hill et al., Nucleic Acids Research 49:20 (2021), and Lima and Lanza, Virus 13(11):2160 (2021), in particular in Table 1 therein). In some embodiments, a polynucleotide encodes, N-terminal and/or C-terminal and connected to a 2A (or 2A-like) peptide, a linker sequence. An example of such a linker sequence is the amino acid sequence GSG or the amino acid sequence SGSG.

In any of the presently disclosed embodiments, a self-cleaving polypeptide encoded by a polynucleotide of this disclosure comprises a P2A, a T2A, an E2A, or a F2A. In certain embodiments, a self-cleaving polypeptide is fused to a linker (e.g. GSG), such as at the N-terminus of the self-cleaving polypeptide.

In some embodiments, a polynucleotide encodes a furin cleavage site. A furin cleavage sequence (also referred-to as a furin recognition site) can have a minimal cleavage site of R-X-X-R (SEQ ID NO.:53). In some embodiments, a furin cleavage sequence has a minimal cleavage site of R-X-K/R-R (SEQ ID NO.:23). In some embodiments, a furin cleavage sequence has a minimal cleavage site of RAKR (SEQ ID NO.:54) or RARR (SEQ ID NO.:55). In certain embodiments, a polynucleotide encoding a furin cleavage site is disposed upstream of (5′ to) a polynucleotide encoding a self-cleaving peptide (with optional linker). In particular embodiments, a polynucleotide comprises, in 5′ to 3′ direction, a polynucleotide encoding a fusion protein of the present disclosure, a polynucleotide encoding a furin cleavage site, a polynucleotide encoding (an optional linker and) a self-cleaving polypeptide. Without wishing to be bound by theory, such an arrangement can avoid, reduce, or minimize unnecessary amino acids at the C-terminal end of a fusion protein (e.g. C-terminal to an IL-2Rγ or IL-2Rγ intracellular domain), and may provide improved transduction of an IL-2 signal by the fusion protein in a host cell.

In any of the embodiments described herein, a polynucleotide of the present disclosure (e.g., a fusion protein-encoding polynucleotide or polynucleotide encoding a marker or polynucleotide encoding an antigen-binding protein) may be codon-optimized for expression in a host cell (see, e.g, Scholten et al., Clin. Immunol. 119:135-145 (2006). Codon optimization can be performed using known techniques and tools, e.g., using the GenScript® OptimumGene™ tool, or the GeneArt™/GeneOptimizer™ tools. Codon-optimized sequences include sequences that are partially codon-optimized (i.e., one or more of the codons is optimized for expression in the host cell) and those that are fully codon-optimized.

In certain embodiments, polynucleotide encoding a fusion protein or an antigen-binding protein further comprises a polynucleotide encoding a leader or signal sequence, also called a signal peptide. Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. Signal peptides can be at the N-terminal or C-terminal end of an encoded polypeptide. An exemplary leader amino acid sequence is from GM-CSF. Other leader amino acid sequences include those from TGFβRI (SEQ ID NO.:21), TGFβRII (SEQ ID NO.:22), CD8α, CD8β, murine IgG, kappa light chain, or the like. Certain signal peptides and characteristics of these are described in Owji et al., European Journal of Cell Biology 97(6):422-441 (2018), and in Ling et al. Front. Immunol. (2020) doi.org/10.3389/fimmu.2020.604318; the signal peptides of which are incorporated herein by reference.

In further aspects, expression constructs are provided, wherein the expression constructs comprise a polynucleotide of the present disclosure operably linked to an expression control sequence (e.g., a promoter). An exemplary promoter sequence includes an EF1 promoter or a MSCV U3 promoter or a MNDU3 promoter. In certain embodiments, the expression construct is comprised in a vector. An exemplary vector may comprise a polynucleotide capable of transporting another polynucleotide to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding fusion proteins as described herein) are co-administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a vector selected from lentiviral vector or a γ-retroviral vector). Viral vectors include retrovirus, adenovirus (e.g., adeno-associated viruses), parvovirus, coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types I and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing CAR transgenes are known in the art and have been previous described, for example, in: U.S. Pat. No. 8,119,772; Walchli et al., PLoS One 6:327930, 2011; Zhao el al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Construction of an expression vector that is used for genetically engineering and producing a fusion protein of interest can be accomplished by using any suitable molecular biology engineering techniques known in the art. To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the immunogen.

In certain embodiments, polynucleotides of the present disclosure are used to transfect/transduce a host cell (e.g., a T cell). A host cell encoding and/or expressing a fusion protein as disclosed herein is, in certain embodiments, useful in adoptive transfer therapy (e.g., targeting a cancer antigen or targeting an adoptively transferred cell that expresses a tag peptide). Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to fusion proteins of the present disclosure.

In certain embodiments, the host cell is a hematopoietic progenitor cell or a human immune system cell. A “hematopoietic progenitor cell”, as referred to herein, is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻ CD117⁺ phenotype or those found in the thymus (referred to as progenitor thymocytes).

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include a CD4⁺ T cell, a CD8⁺ T cell, a CD4⁻ CD8⁻ double negative T cell, a γδ T cell, a regulatory T cell, a stem cell memory T cell, a natural killer cell (e.g., a NK cell or a NK-T cell), a B cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces T cell receptors (TCRs), though it will be understood that a T cell in which expression of a native TCR is (e.g., artificially) suppressed or abrogated is still a T cell. T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}).

Effector T cells (T_{E}) refer to antigen-experienced CD8⁺ cytotoxic T lymphocytes that have decreased expression of CD62L, CCR7, CD28, and are positive for granzyme and perforin as compared to T_{CM}. Helper T cells (T_{H}) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺ CD25⁺ (Foxp3⁺) regulatory T cells and Treg17 cells, as well as Tr1, Th3, CD8⁺ CD28⁻, and Qa-1 restricted T cells.

“Cells of T cell lineage” refer to cells that show at least one phenotypic characteristic of a T cell, or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺ CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺ CD8⁺ double positive; single positive CD4⁺ or CD8⁺; TCRαβ or TCR γδ; or mature and functional or activated T cells.

In certain embodiments, the immune system cell is a CD4+ T cell, a CD8+ T cell, a CD4− CD8− double negative T cell, a γδ T cell, a natural killer cell (e.g., NK cell or NK-T cell), a dendritic cell, a B cell, or any combination thereof. In certain embodiments, the immune system cell is a CD4+ T cell. In certain embodiments, the T cell is a naïve T cell, a central memory T cell, an effector memory T cell, a stem cell memory T cell, or any combination thereof.

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In any of the foregoing embodiments, a host cell that comprises a heterologous polynucleotide encoding a fusion protein can be an immune cell which is modified to reduce or eliminate expression of one or more endogenous genes that encode a polypeptide product selected from a TGFβR1 gene locus, a TGFβR2 gene locus, a PD-1 gene locus, a CTLA4 gene locus, a LAT gene locus, a TIM-3 gene locus, a PD-L1 gene locus, a TIGIT gene locus, an A2AR gene locus, a Fas locus, a FasL gene locus, a B7-H3 gene locus, a B7-H4 gene locus, an IDO gene locus, a VISTA gene locus, a SIGLEC7 gene locus, a SIGLEC9 gene locus, a TRAC gene locus, a TRBC gene locus, a T cell receptor gene locus, a MHC (e.g. HLA) gene locus, a CBLB gene locus, a RASA2 gene locus, a UBASH3A gene locus, a CISH gene locus, or any combination thereof.

Without wishing to be bound by theory, certain endogenously expressed immune cell proteins may downregulate the immune activity of a modified immune host cell (e.g., PD-1, LAG-3, CTLA4, TIGIT, CBLB, RASA2, UBASH3A, CISH, Fas), or may compete with a fusion protein of the present disclosure for expression by the host cell and/or binding to a TGFβ and/or for may cause undesired TGFβ signaling (e.g., TGFβR1 and/or TGFβRII), or may interfere with the binding activity of an antigen-binding protein of the present disclosure and interfere with the host cell binding to a target cell that expresses an antigen, or any combination thereof. Further, endogenous proteins (e.g., immune host cell proteins, such as an HLA) expressed on a donor immune cell to be used in a cell transfer therapy may be recognized as foreign by an allogeneic recipient, which may result in elimination or suppression of the donor immune cell by the allogeneic recipient.

Accordingly, decreasing or eliminating expression or activity of such endogenous genes or proteins can improve the activity, tolerance, and persistence of the host cells in an autologous or allogeneic host setting, and can allow universal administration of the cells (e.g., to any recipient regardless of HLA type). In certain embodiments, a modified host immune cell is a donor cell (e.g., allogeneic) or an autologous cell. In certain embodiments, a modified immune host cell of this disclosure comprises a chromosomal gene knockout of one or more of a gene that encodes PD-1, LAG-3, CTLA4, TIM3, TIGIT, an HLA component (e.g., a gene that encodes an α1 macroglobulin, an a2 macroglobulin, an 3 macroglobulin, a β1 microglobulin, or a β2 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24):5697 (2012); and Torikai et al., Blood 122(8):1341 (2013) the gene editing techniques, compositions, and adoptive cell therapies of which are herein incorporated by reference in their entirety), TGFβR1, TGFβR2, LAT, A2AR, Fas, FasL, B7-H3, B7-H4, IDO, VISTA, SIGLEC7, SIGLEC9, TRAC, TRBC, CBLB, RASA2, UBASH3A, and/or CISH. As used herein, the term “chromosomal gene knockout” refers to a genetic alteration in a host cell that prevents production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais el al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337.816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Examples of gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

Alternative Cas nucleases may be used, including but not limited to, Cas 12, Cas 13, and Cas 14 nucleases, and variants thereof. For example, Cas nucleases disclosed in WO 2019/178427, which is hereby incorporated by reference in its entirety (including the Cas nucleases, CRISPR-Cas systems, and related methods disclosed therein), may be utilized.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perer et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from PD-1, LAG3, TIM3, CTLA4, TIGIT, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat el al., Nucleic Acids Res. 31:2952-62, 2003; Chevalier el al., Molec. Cell 10:895-905, 2002; Ashworth el al., Nature 441:656-659, 2006; Paques el al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (e.g., of PD-1, TIM3, LAG3, CTLA4, TIGIT, an HLA component, a TCR component, a TGFβRI, a a TGFβRII, or any combination thereof) in the host immune cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

Any of the foregoing gene-editing techniques can be used to introduce a polynucleotide of the present disclosure (e.g., encoding a fusion protein) into a host cell genome. In some embodiments, a heterologous polynucleotide is introduced into a locus encoding an endogenous TCR component, HLA component, PD-1, LAG-3, CTLA4, TIM3, or TIGIT, or a “safe harbor” locus such as Rosa26, AAVS1, CCR5, or the like.

In certain embodiments, a host cell (e.g., immune cell) of the present disclosure is engineered so that expression of a fusion protein or an antigen-binding protein is modulated (e.g., controlled) by binding of the host cell to an antigen that is not the same antigen as the antigen to which the antigen-binding protein specifically binds, or, for the fusion protein, that is not a TGFβ.

For example, a host cell can comprise (i) a polynucleotide encoding an engineered (i.e., synthetic) Notch receptor comprising (a) an extracellular component comprising a binding domain that binds to an antigen, which is a different antigen than the antigen to which the antigen-binding protein binds, (b) a Notch core domain, or a functional portion or variant thereof; and (c) an intracellular component comprising a transcriptional factor (i.e., a polypeptide capable of activating or increasing, or inhibiting, repressing or reducing, transcription of a target nucleotide sequence (e.g., a gene) or set of target nucleotide sequences); and (ii) the heterologous polynucleotide encoding an antigen-binding protein as disclosed herein and comprising an expression control sequence that can be recognized or bound by the transcriptional factor, wherein binding of the engineered Notch receptor to antigen leads to release of the transcriptional factor from the engineered Notch receptor (e.g., by protease-driven cleavage), which can, in turn, drive transcription of the fusion protein. See, e.g., Morsut et al., Cell 164:780-791 (2016) and PCT Published Application No. WO 2016/138034A1, which synthetic Notch constructs are incorporated herein by reference. Briefly, such “logic-gated” expression systems may be useful to modulate expression of an antigen-binding protein of this disclosure so that the expression occurs only, or preferentially, when the host cell encounters a first antigen (i.e., that can be bound by the synthetic Notch receptor) that is only expressed by, or is principally expressed by, or has a higher expression level on cancer cells as compared to healthy cells. Such embodiments may reduce “on-target off-tissue” recognition by a fusion protein in circumstances where the target recognized by the antigen-binding protein is expressed by healthy cells.

In other aspects, kits are provided comprising (a) a vector or an expression construct as described herein and (b) reagents for transducing the vector or the expression construct into a host cell.

### Uses

The present disclosure also provides methods for treating a disease or condition, wherein the methods comprise administering to a subject in need thereof an effective amount of a fusion protein, polynucleotide, vector, host cell, composition, or unit dose of the present disclosure. In some embodiments, the disease or condition expresses or is otherwise associated with the target (e.g., antigen) and the method comprises administering a fusion-protein-expressing host cell that further expresses an antigen-binding protein. In some embodiments.

As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre malignant cells, as well as non-neoplastic or non-malignant disorders.

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

In certain embodiments, a cancer treatable according to the presently disclosed methods and uses comprises a carcinoma, a sarcoma, a glioma, a lymphoma, a leukemia, a myeloma, or any combination thereof. In certain embodiments, cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

In certain embodiments, a cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenstrom's macroglobulinemia, CD37+ dendritic cell lymphoma, lymphoplasmacytic lymphoma, splenic marginal zone lymphoma, extranodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

In certain embodiments, the cancer comprises a solid tumor. In some embodiments, the solid tumor is a sarcoma or a carcinoma. In certain embodiments, the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma, angiosarcoma (vascular sarcoma); Kaposi's sarcoma; liposarcoma; pleomorphic sarcoma; or synovial sarcoma. In certain embodiments, the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carcinoma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma). In certain embodiments, the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In any of the presently disclosed embodiments, the host cell is an allogeneic cell, a syngeneic cell, or an autologous cell. Typically, the host cell will further express or encode an antigen-binding protein. Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a fusion protein as described herein is administered intravenously, intraperitoneally, intratumorally, into the bone marrow, into a lymph node, or into the cerebrospinal fluid so as to encounter the tagged cells to be ablated. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In any of the above embodiments, methods of the present disclosure comprise administering a host cell expressing a fusion protein of the present disclosure. Typically, the host cell will further express or encode an antigen-binding protein. The amount of cells in a composition is at least one cell (for example, one fusion protein-modified CD8⁺ T cell subpopulation; one fusion protein-modified CD4⁺ T cell subpopulation) or is more typically greater than 10² cells, for example, up to 10⁶, up to 10⁷, up to 10⁸ cells, up to 10⁹ cells, or more than 10¹⁰ cells, such as about 10¹¹ cells/m². In certain embodiments, the cells are administered in a range from about 10⁵ to about 10¹¹ cells/m², preferably in a range of about 10⁵ or about 10⁶ to about 10⁹ or about 10¹⁰ cells/m². The number of cells will depend upon the ultimate use for which the composition is intended as well the type of cells included therein. For example, cells modified to contain a fusion protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁵, 10⁶, 10⁷, 10⁸, 10⁹, 10¹⁰, or 10¹¹ cells.

Unit doses are also provided herein which comprise a host cell (e.g., a modified immune cell comprising a polynucleotide of the present disclosure) or host cell composition of this disclosure. Typically, the host cell will further express or encoden an antigen-binding protein. In certain embodiments, a unit dose comprises (i) a composition comprising at least about 30% (e.g., including 30% or more), at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio (e.g., such as a 1:1 ratio), wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% modified CD4⁺ T cells, combined with (ii) a composition comprising at least about 70% modified CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose comprises equal, or approximately equal numbers of engineered CD45RA⁻ CD3⁺ CD8⁺ and engineered CD45RA⁻ CD3⁺ CD4⁺ T_{M} cells.

Also contemplated are pharmaceutical compositions that comprise fusion proteins or cells expressing or encoding a fusion protein as disclosed herein, and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the fusion protein-expressing cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with the target (e.g., antigen). Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a desired, fusion protein-expressing T-cell composition to a subject as an adoptive immunotherapy. In certain embodiments, the host cell comprises a hematopoietic progenitor cell or a human immune cell. In certain embodiments, the immune system cell comprises a CD4⁺ T cell, a CD8⁺ T cell, a CD4⁻ CD8⁻ double-negative T cell, a γδ T cell, a natural killer cell, a dendritic cell, or any combination thereof. In certain embodiments, the immune system cell comprises a naïve T cell, a central memory T cell, a stem cell memory T cell, an effector memory T cell, or any combination thereof. In particular embodiments, the cell comprises a CD4⁺ T cell. In particular embodiments, the cell comprises a CD8⁺ T cell.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., fusion protein-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a recombinant host cell as described herein is administered to the subject, which may be administered at intervals between administrations of about two to about four weeks or more. In certain embodiments, the plurality of unit doses are administered at intervals between administrations of about two, three, four, five, six, seven, eight, or more weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

An effective amount of a pharmaceutical composition (e.g., host cell, fusion protein, unit dose, or composition) refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The level of a CTL immune response may be determined by any one of numerous immunological methods described herein and routinely practiced in the art. The level of a CTL immune response may be determined prior to and following administration of any one of the herein described fusion proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, PA), pages 1127-50, and references cited therein).

Target (e.g., antigen)-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a fusion protein or cell as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as sealed ampoules or vials. Such containers may be frozen to preserve the stability of the formulation until. In certain embodiments, a unit dose comprises a recombinant host cell as described herein at a dose of about 10⁵ cells/M² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine in the art and may be performed using samples obtained from a subject before and after treatment.

In further aspects, kits are provided that comprise (a) a host cell, (b) a composition, or (c) a unit dose as described herein.

Methods according to this disclosure may further include administering one or more additional agents to treat the disease or disorder in a combination therapy. For example, in certain embodiments, a combination therapy comprises administering a fusion protein (or an engineered host cell expressing the same) with (concurrently, simultaneously, or sequentially) an immune checkpoint inhibitor. In some embodiments, a combination therapy comprises administering fusion protein of the present disclosure (or an engineered host cell expressing the same) with an agonist of a stimulatory immune checkpoint agent. In further embodiments, a combination therapy comprises administering a fusion protein of the present disclosure (or an engineered host cell expressing the same) with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GAL9, KIR, PVR1G (CD112R), PVRL2, adenosine, A2aR, immunosuppressive cytokines (e.g., IL-10, IL-4, IL-IRA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a composition of the present disclosure (e.g., a fusion protein, polynucleotide, vector, an host cell, or pharmaceutical composition) with one or more inhibitor of any one of the following immune suppression components, singly or in any combination. In certain embodiments, a composition is used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MED10680 (formerly AMP-514), AMP-224, BMS-936558, or any combination thereof. In further embodiments, a composition is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof. In certain embodiments, a composition is used in combination with a LAG3 inhibitor, such as LAG525, IMP321, IMP701, 9H12, BMS-986016, or any combination thereof. In certain embodiments, a composition is used in combination with an inhibitor of CTLA4. In particular embodiments, a composition is used in combination with a CTLA4 specific antibody or binding fragment thereof, such as ipilimumab, tremelimumab, CTLA4-Ig fusion proteins (e.g., abatacept, belatacept), or any combination thereof. In certain embodiments, a composition is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1. In certain embodiments, a composition is used in combination with an inhibitor of CD244. In certain embodiments, a composition is used in combination with an inhibitor of BLTA, HVEM, CD160, or any combination thereof. Anti CD-160 antibodies are described in, for example, PCT Publication No. WO 2010/084158. In certain embodiments, a composition is used in combination with an inhibitor of TIM3. In certain embodiments, a composition is used in combination with an inhibitor of Gal9. In certain embodiments, a composition is used in combination with an inhibitor of adenosine signaling, such as a decoy adenosine receptor. In certain embodiments, a composition is used in combination with an inhibitor of A2aR. In certain embodiments, a composition is used in combination with an inhibitor of KIR, such as lirilumab (BMS-986015). In certain embodiments, a composition is used in combination with an inhibitor of an inhibitory cytokine (typically, a cytokine other than TGFβ) or Treg development or activity. In certain embodiments, a composition is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013: Apr. 6-10, 2013), 1-methyl-tryptophan (1-MT)-tira-pazamine, or any combination thereof. In certain embodiments, a composition is used in combination with an arginase inhibitor, such as N(omega)-Nitro-L-arginine methyl ester (L-NAME), N-omega-hydroxy-nor-l-arginine (nor-NOHA), L-NOHA, 2(S)-amino-6-boronohexanoic acid (ABH), S-(2-boronoethyl)-L-cysteine (BEC), or any combination thereof. In certain embodiments, a composition of the present disclosure (or an engineered host cell expressing the same) is used in combination with an inhibitor of VISTA, such as CA-170 (Curis, Lexington, Mass.). In certain embodiments, a composition is used in combination with an inhibitor of TIGIT such as, for example, COM902 (Compugen, Toronto, Ontario Canada), an inhibitor of CD155, such as, for example, COM701 (Compugen), or both. In certain embodiments, a composition is used in combination with an inhibitor of PVR1G, PVRL2, or both. Anti-PVRIG antibodies are described in, for example, PCT Publication No. WO 2016/134333. Anti-PVRL2 antibodies are described in, for example, PCT Publication No. WO 2017/021526. In certain embodiments, a composition is used in combination with a LAIR1 inhibitor. In certain embodiments, a composition is used in combination with an inhibitor of CEACAM-1, CEACAM-3, CEACAM-5, or any combination thereof. In certain embodiments, a composition is used in combination with an agent that increases the activity (i.e., is an agonist) of a stimulatory immune checkpoint molecule. For example, a composition of the present disclosure can be used in combination with a CD137 (4-1BB) agonist (such as, for example, urelumab), a CD134 (OX-40) agonist (such as, for example, MEDI6469, MED16383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a composition with one or more agonist of a stimulatory immune checkpoint molecule, including any of the foregoing, singly or in any combination.

In certain embodiments, a combination therapy comprises a composition and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a composition and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer or non-inflamed solid tumor in a subject are well-known to those of ordinary skill in the art.

In certain embodiments, a combination therapy method comprises administering composition and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines can be used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the fusion proteins or cells expressing the same (or vectors or polynucleotides) of this disclosure.

In certain embodiments, the subject is receiving, has received, or will receive one or more of: (i) chemotherapy; (ii) radiation therapy; (iii) an inhibitor of an immune suppression component; (iv) an agonist of a stimulatory immune checkpoint agent; (v) RNAi; (vi) a cytokine; (vii) a surgery; (viii) a monoclonal antibody and/or an antibody-drug conjugate; or (ix) any combination of (i)-(viii), in any order.

Also provided herein are uses of any of the presently disclosed fusion proteins, polynucleotides, vectors, host cells, compositions, or unit doses, for use in the treatment of a disease or disorder in a subject, wherein the disease or condition is optionally characterized by the presence of an antigen (e.g. that is bound by the binding domain of an antigen-binding protein such as expressed in a host cell)(e.g., any target as disclosed herein).

Also provided herein are uses of any of the presently disclosed fusion proteins, polynucleotides, vectors, host cells, compositions, or unit doses, for use in the manufacture of a medicament for the treatment of a disease or condition in a subject.

The present disclosure also provides the following non-limiting enumerated Embodiments.

Embodiment 1. A fusion protein comprising:
(i) an extracellular component comprising an extracellular domain of a transforming growth factor beta receptor (TGFβR) polypeptide, or a portion or variant thereof that is capable of binding to a TGFβ polypeptide;
(ii) an intracellular component comprising an intracellular portion of an interleukin-2 receptor (IL-2R) polypeptide; and
(iii) a transmembrane component disposed between the extracellular component of (i) and the intracellular component of (ii),
wherein the fusion protein, when expressed by a host cell and bound to the TGFβ polypeptide, is capable of contributing to an IL-2 signal in the host cell.

Embodiment 2. The fusion protein of Embodiment 1, wherein the TGFβR polypeptide comprises a TGFβRI polypeptide or a TGFβR2 polypeptide.

Embodiment 3. The fusion protein of Embodiment 1 or 2, wherein the IL-2R polypeptide comprises an IL-2Rβ polypeptide, an IL-2Rγ polypeptide, or both.

Embodiment 4. A fusion protein comprising:
(i) an extracellular component comprising an extracellular domain of a transforming growth factor beta receptor 1 (TGFβR1) polypeptide, or a portion or variant thereof that is capable of binding to a TGFβ polypeptide;
(ii) an intracellular component comprising an intracellular portion of an interleukin-2 receptor beta (IL-2Rβ) polypeptide; and
(iii) a transmembrane component disposed between the extracellular component of (i) with the intracellular component of (ii),
wherein the fusion protein, when expressed by a host cell and bound to the TGFβ polypeptide, is capable of contributing to an IL-2 signal in the host cell.

Embodiment 5. A fusion protein comprising:
(i) an extracellular component comprising an extracellular domain of a transforming growth factor beta-receptor 2 (TGFβR2) polypeptide, or a portion or variant thereof that is capable of binding to a TGFβ polypeptide;
(ii) an intracellular component comprising an intracellular portion of an interleukin-2 receptor beta (IL-2Rβ) polypeptide; and
(iii) a transmembrane component disposed between the extracellular component of (i) with the intracellular component of (ii),
wherein the fusion protein, when expressed by a host cell and bound to the TGFβ polypeptide, is capable of contributing to an IL-2 signal in the host cell.

Embodiment 6. A fusion protein comprising:
(i) an extracellular component comprising an extracellular domain of a transforming growth factor beta receptor 2 (TGFβR2) polypeptide, or a portion or variant thereof that is capable of binding to a TGFβ polypeptide;
(ii) an intracellular component comprising an intracellular portion of an interleukin-2 receptor gamma (IL-2Rγ) polypeptide; and
(iii) a transmembrane component disposed between the extracellular component of (i) with the intracellular component of (ii),
wherein the fusion protein, when expressed by a host cell and bound to the TGFβ polypeptide, is capable of contributing to an IL-2 signal in the host cell.

Embodiment 7. A fusion protein comprising:
(i) an extracellular component comprising an extracellular domain of a transforming growth factor beta receptor 1 (TGFβR1) polypeptide, or a portion or variant thereof that is capable of binding to a TGFβ polypeptide;
(ii) an intracellular component comprising an intracellular portion of an interleukin-2 receptor gamma (IL-2Rγ) polypeptide; and
(iii) a transmembrane component disposed between the extracellular component of (i) with the intracellular component of (ii),
wherein the fusion protein, when expressed by a host cell and bound to the TGFβ polypeptide, is capable of contributing to an IL-2 signal in the host cell.

Embodiment 8. The fusion protein of any one of Embodiments 1-7, wherein the transmembrane component comprises a transmembrane domain from IL-2Rβ.

Embodiment 9. The fusion protein of any one of Embodiments 1-8, wherein the transmembrane component comprises a transmembrane domain from IL-2Rγ.

Embodiment 10. The fusion protein of any one of Embodiments 1-9, wherein the transmembrane component comprises a transmembrane domain from TGFβR1.

Embodiment 11. The fusion protein of any one of Embodiments 1-10, wherein the transmembrane component comprises a transmembrane domain from TGFβR2.

Embodiment 12. A fusion protein comprising:
(i) an extracellular component comprising an extracellular domain of a transforming growth factor beta receptor 1 (TGFβR1) polypeptide, or a portion or variant thereof that is capable of binding to a TGFβ polypeptide;
(ii) an intracellular component comprising an intracellular portion of an interleukin-2 receptor beta (IL-2Rβ) polypeptide, wherein the intracellular component optionally comprises an IL-2Rβ intracellular domain; and
(iii) a transmembrane component disposed between the extracellular component of (i) with the intracellular component of (ii), wherein the transmembrane component optionally comprises an IL-2Rβ transmembrane domain,
wherein the fusion protein, when expressed by a host cell and bound to the TGFβ polypeptide, is capable of contributing to an IL-2 signal in the host cell.

Embodiment 13. A fusion protein comprising:
(i) an extracellular component comprising an extracellular domain of a transforming growth factor beta receptor 2 (TGFβR2) polypeptide, or a portion or variant thereof that is capable of binding to a TGFβ polypeptide;
(ii) an intracellular component comprising an intracellular portion of an interleukin-2 receptor beta (IL-2Rβ) polypeptide, wherein the intracellular component optionally comprises an IL-2Rβ intracellular domain; and
(iii) a transmembrane component disposed between the extracellular component of (i) with the intracellular component of (ii), wherein the transmembrane component optionally comprises an IL-2Rβ transmembrane domain,
wherein the fusion protein, when expressed by a host cell and bound to the TGFβ polypeptide, is capable of contributing to an IL-2 signal in the host cell.

Embodiment 14. A fusion protein comprising:
(i) an extracellular component comprising an extracellular domain of a transforming growth factor beta receptor 1 (TGFβR1) polypeptide, or a portion or variant thereof that is capable of binding to a TGFβ polypeptide;
(ii) an intracellular component comprising an intracellular portion of an interleukin-2 receptor beta (IL-2Rβ) polypeptide, wherein the intracellular component optionally comprises a IL-2Rβ transmembrane domain; and
(iii) a transmembrane component disposed between the extracellular component of (i) with the intracellular component of (ii), wherein the transmembrane component optionally comprises a TGFβR1 transmembrane domain, wherein the fusion protein, when expressed by a host cell and bound to the TGFβ polypeptide, is capable of contributing to an IL-2 signal in the host cell.

Embodiment 15. A fusion protein comprising:
(i) an extracellular component comprising an extracellular domain of a transforming growth factor beta receptor 2 (TGFβR2) polypeptide, or a portion or variant thereof that is capable of binding to a TGFβ polypeptide;
(ii) an intracellular component comprising an intracellular portion of an interleukin-2 receptor beta (IL-2Rβ) polypeptide, wherein the intracellular component optionally comprises a IL-2Rβ intracellular domain; and
(iii) a transmembrane component disposed between the extracellular component of (i) with the intracellular component of (ii), wherein the transmembrane component optionally comprises a TGFβR2 transmembrane domain,
wherein the fusion protein, when expressed by a host cell and bound to the TGFβ polypeptide, is capable of contributing to an IL-2 signal in the host cell.

Embodiment 16. A fusion protein comprising:
(i) an extracellular component comprising an extracellular domain of a transforming growth factor beta receptor 2 (TGFβR2) polypeptide, or a portion or variant thereof that is capable of binding to a TGFβ polypeptide;
(ii) an intracellular component comprising an intracellular portion of an interleukin-2 receptor gamma (IL-2Rγ) polypeptide, wherein the intracellular component optionally comprises a IL-2Rγ intracellular domain; and
(iii) a transmembrane component disposed between the extracellular component of (i) with the intracellular component of (ii), wherein the transmembrane component optionally comprises an IL-2Rγ transmembrane domain,
wherein the fusion protein, when expressed by a host cell and bound to the TGFβ polypeptide, is capable of contributing to an IL-2 signal in the host cell.

Embodiment 17. A fusion protein comprising: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor gamma (IL-2Rγ) polypeptide, wherein the intracellular component optionally comprises a IL-2Rγ intracellular domain; and (iii) a transmembrane component disposed between the extracellular component of (i) with the intracellular component of (ii), wherein the transmembrane component optionally comprises an IL-2Rγ transmembrane domain, wherein the fusion protein, when expressed by a host cell and bound to the TGFβ polypeptide, is capable of contributing to an IL-2 signal in the host cell.

Embodiment 18. A fusion protein comprising:
(i) an extracellular component comprising an extracellular domain of a transforming growth factor beta receptor 2 (TGFβR2) polypeptide, or a portion or variant thereof that is capable of binding to a TGFβ polypeptide;
(ii) an intracellular component comprising an intracellular portion of an interleukin-2 receptor gamma (IL-2Rγ) polypeptide, wherein the intracellular component optionally comprises a IL-2Rγ intracellular domain; and
(iii) a transmembrane component disposed between the extracellular component of (i) with the intracellular component of (ii), wherein the transmembrane component optionally comprises an TGFβR2 transmembrane domain,
wherein the fusion protein, when expressed by a host cell and bound to the TGFβ polypeptide, is capable of contributing to an IL-2 signal in the host cell.

Embodiment 19. A fusion protein comprising:
(i) an extracellular component comprising an extracellular domain of a transforming growth factor beta receptor 1 (TGFβR1) polypeptide, or a portion or variant thereof that is capable of binding to a TGFβ polypeptide;
(ii) an intracellular component comprising an intracellular portion of an interleukin-2 receptor gamma (IL-2Rγ) polypeptide, wherein the intracellular component optionally comprises a IL-2Rγ intracellular domain; and
(iii) a transmembrane component disposed between the extracellular component of (i) with the intracellular component of (ii), wherein the transmembrane component optionally comprises an TGFβRI transmembrane domain,
wherein the fusion protein, when expressed by a host cell and bound to the TGFβ polypeptide, is capable of contributing to an IL-2 signal in the host cell.

Embodiment 20. The fusion protein of any one of Embodiments 1-19, wherein: (1) (i) the extracellular component has at least 90%, at least 91%, at least 92%, at least 93%, at least 94%, at least 95%, at least 96%, at least 97%, at least 98%, or at least 99% identity to, or comprises or consists of, the amino acid sequence set forth in SEQ ID NO.:39 or 41; (ii) the transmembrane component has at least 90%, at least 91%, at least 92%, at least 93%, at least 94%, at least 95%, at least 96%, at least 97%, at least 98%, or at least 99% identity to, or comprises or consists of, the amino acid sequence set forth in: SEQ ID NO.:40; SEQ ID NO.:42; SEQ ID NO.:43; or SEQ ID NO.:45; and (iii) the intracellular component has at least 90%, at least 91%, at least 92%, at least 93%, at least 94%, at least 95%, at least 96%, at least 97%, at least 98%, or at least 99% identity to, or comprises or consists of, the amino acid sequence set forth in SEQ ID NO.:44 or 46, and wherein, optionally, the fusion protein comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOS.:5, 6, 9, 10, 13, 14, 17, and 18; and/or (2) wherein the fusion protein comprises one or more amino acid substitutions, insertions, and/or deletions to reduce or prevent the fusion protein from forming a protein dimer with a human TGFβRI or a human TGFβR2, wherein, optionally, the one or more amino acid substitutions, insertions, and/or deletions provide: (i) a variant of SEQ ID NO.:47, wherein the variant comprises from one to ten amino acid insertions, or from one to ten amino acid deletions; (ii) one, two, three, four, five, six, seven, eight, nine, ten, or more amino acids added to the N-terminus and/or to the C-terminus of SEQ ID NO.:47; (iii) a variant of SEQ ID NO.:48, wherein the variant comprises from one to ten amino acid insertions, or from one to ten amino acid deletions; (iv) one, two, three, four, five, six, seven, eight, nine, ten, or more added to the N-terminus and/or to the C-terminus of SEQ ID NO.:48.

Embodiment 21. The fusion protein of any one Embodiments 1-20, wherein the IL-2 signal comprises, results in, provides, or promotes any one or more of (i)-(vii): (i) phosphorylation of the intracellular portion of the IL-2R polypeptide by JAK1 or JAK3; (ii) phosphorylation in the host cell of any one or more of: PI3K; Akt; STAT; STAT5A; STAT5B; MEK; SHC1; MEK1; MEK2; ERK1; ERK2; and STAT3; (iii) STAT5-mediated transcription; (iv) recruitment of GRB2 and SOS; (v) GTP loading of RAS; (vi) activation of the Raf-ERK MAPK cascade; (vii) activation of mTORC1 and/or HIF1α/HIF1β.

Embodiment 22. The fusion protein of any one Embodiments 1-21, wherein, when the fusion protein is expressed by a host cell and the fusion protein binds to the TGFβ polypeptide, the host cell performs one or more of the following: proliferation; growth; expression of an effector molecule (e.g., a T cell receptor); glycolytic metabolism; expression of protein (e.g., MYC, SLC7A5, or both); and biosynthesis of cholesterol.

Embodiment 23. A fusion protein comprising or consisting of the amino acid sequence set forth in any one of SEQ ID NOs.:5, 6, 9, 10, 13, 14, 17, and 18.

Embodiment 24. A fusion protein comprising or consisting of the amino acid sequence set forth in any one of SEQ ID NOs.:7, 11, 15, and 19.

Embodiment 25. A polynucleotide encoding the fusion protein of any one of Embodiments 1-24.

Embodiment 26. A polynucleotide encoding the fusion protein of any one of Embodiments 1-24, wherein the encoded fusion protein further comprises, N-terminal to and connected to the extracellular component, a signal peptide.

Embodiment 27. The polynucleotide of Embodiment 26, wherein the signal peptide comprises or consists of the amino acid sequence set forth in SEQ ID NO.:21 or 22.

Embodiment 28. A polynucleotide encoding (1) a first fusion protein of any one of Embodiments 1-24 and (2) a second fusion protein of any one of Embodiments 1-24.

Embodiment 29. The polynucleotide of Embodiment 28, wherein an amino acid sequence of the first fusion protein is different than an amino acid sequence of the second fusion protein,
wherein, optionally:
(a) the first fusion protein comprises an extracellular domain of a TGFβR1 polypeptide, or a portion or variant thereof that is capable of binding to a TGFβ polypeptide, and the second fusion protein comprises an extracellular domain of a TGFβR2 polypeptide, or a portion or variant thereof that is capable of binding to a TGFβ polypeptide; and/or
(b) the first fusion protein comprises an intracellular portion of an IL-2Rγ polypeptide, preferably an IL-2Rγ intracellular domain, and the second fusion protein comprises an intracellular portion of an IL-2Rβ polypeptide, preferably an IL-2Rβ intracellular domain, or (c) the first fusion protein comprises an intracellular portion of an IL-2Rβ polypeptide, preferably an IL-2Rβ intracellular domain, and the second fusion protein comprises an intracellular portion of an IL-2Rγ polypeptide, preferably an IL-2Rγ intracellular domain; and/or
(d) the first fusion protein comprises an IL-2Rβ transmembrane domain, an IL-2Rγ transmembrane domain, a TGFβR1 transmembrane domain, or a TGFβR2 transmembrane domain, and the second fusion protein comprises an IL-2Rβ transmembrane domain, an IL-2Rγ transmembrane domain, a TGFβR1 transmembrane domain, or a TGFβR2 transmembrane domain.

Embodiment 30. The polynucleotide of Embodiment 28 or 29, further comprising, disposed between a nucleotide sequence encoding the first fusion protein and a nucleotide sequence encoding the second fusion protein:
(i) a nucleotide sequence encoding a furin cleavage site;
(ii) a nucleotide sequence encoding a self-cleaving polypeptide (e.g., P2A, T2A, E2A, F2A); or
(iii) both (i) and (ii), wherein, optionally, (i) is disposed 5′ to (i.e. upstream of) (ii).

Embodiment 31. The polynucleotide of any one of Embodiments 25-30, which is codon optimized for expression in a host cell, wherein the host cell is optionally a human host cell, further optionally a human immune system cell, still further optionally a human T cell (e.g. a CD4+ T cell, a CD8+ T cell, a CD4−CD8− double negative T cell, a γδ T cell, or any combination thereof), a human NK cell, or a human NK-T cell.

Embodiment 32. The polynucleotide of Embodiment 31, which is codon optimized for expression in a T cell (e.g. a CD4+ T cell, a CD8+ T cell, a CD4−CD8− double negative T cell, a γδ T cell, or any combination thereof), a NK cell, or a NK-T cell, wherein the cell is preferably human.

Embodiment 33. The polynucleotide of any one of Embodiments 25-32, wherein the polynucleotide has at least 50%, at least 55%, at least 60%, at least 65%, at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identity to, or comprises or consists of, the amino acid sequence set forth in any one of SEQ ID NOs.:8, 12, 16, and 20.

Embodiment 34. The polynucleotide of any one of Embodiments 25-33, further comprising a nucleotide sequence that encodes an antigen-binding protein.

Embodiment 35. The polynucleotide of Embodiment 34, wherein the antigen-binding protein comprises a TCR, an antigen-binding fragment of a TCR (e.g. a scTv), a scTCR, a CAR, or any combination thereof.

Embodiment 36. The polynucleotide of any one of Embodiments 25-35, further comprising a nucleotide sequence encoding a transduction marker.

Embodiment 37. The polynucleotide of Embodiment 36, wherein the encoded transduction marker comprises EGFRt, CD19t, CD34t, or NGFRt.

Embodiment 38. The polynucleotide of any one of Embodiments 25-37, further comprising a nucleotide sequence encoding a guide RNA, where, optionally, the guide RNA is specific for a TGFβRI gene locus, a TGFβR2 gene locus, a PD-1 gene locus, a CTLA4 gene locus, a LAT gene locus, a TIM-3 gene locus, a PD-L1 gene locus, a TIGIT gene locus, an A2AR gene locus, a Fas locus, a FasL gene locus, a B7-H3 gene locus, a B7-H4 gene locus, an IDO gene locus, a VISTA gene locus, a SIGLEC7 gene locus, a SIGLEC9 gene locus, a TRAC gene locus, a TRBC gene locus, a T cell receptor gene locus, a MHC (e.g. HLA) gene locus, a CBLB gene locus, a RASA2 gene locus, a UBASH3A gene locus, a CISH gene locus, or any combination thereof.

Embodiment 39. The polynucleotide of any one of Embodiments 25-38, further comprising a nucleotide sequence encoding a Cas nuclease, wherein, optionally, the Cas nuclease comprises a Cas9 or a functional variant thereof, a Cas12 or a functional variant thereof, a Cas13 or a functional variant thereof, or a Cas14 or a functional variant thereof.

Embodiment 40. A vector comprising the polynucleotide of any one of Embodiments 25-39, optionally wherein the polynucleotide is operably linked to an expression control sequence.

Embodiment 41. The vector of Embodiment 40, wherein the vector is capable of delivering the polynucleotide to a hematopoietic progenitor cell or a human immune system cell.

Embodiment 42. The vector of Embodiment 41, wherein the human immune system cell comprises a CD4+ T cell, a CD8+ T cell, a CD4−CD8− double negative T cell, a γδ T cell, a natural killer cell, a natural killer T cell, a dendritic cell, or any combination thereof.

Embodiment 43. The vector of Embodiment 42, wherein the T cell comprises a naïve T cell, a central memory T cell, a stem cell memory T cell, an effector memory T cell, or any combination thereof.

Embodiment 44. The vector of any one of Embodiments 40-43, wherein the vector is a viral vector.

Embodiment 45. The vector of Embodiment 44, wherein the viral vector is a lentiviral vector or a γ-retroviral vector.

Embodiment 46. A host cell expressing the fusion protein of any one of Embodiments 1-24.

Embodiment 47. A host cell comprising the polynucleotide of any one of Embodiments 25-39.

Embodiment 48. A host cell comprising the vector of any one of Embodiments 40-45.

Embodiment 49. The host cell of any one of Embodiments 46-48, expressing or encoding (1) a first fusion protein of any one of Embodiments 1-24 and (2) a second fusion protein of any one of Embodiments 1-24.

Embodiment 50. The host cell of Embodiment 49, wherein an amino acid sequence of the first fusion protein is different than an amino acid sequence of the second fusion protein, wherein, optionally:
(a) the first fusion protein comprises an extracellular domain of a TGFβR1 polypeptide, or a portion or variant thereof that is capable of binding to a TGFβ polypeptide, and the second fusion protein comprises an extracellular domain of a TGFβR2 polypeptide, or a portion or variant thereof that is capable of binding to a TGFβ polypeptide; and/or
(b) the first fusion protein comprises an intracellular portion of an IL-2Rγ polypeptide, preferably an IL-2Rγ intracellular domain, and the second fusion protein comprises an intracellular portion of an IL-2Rβ polypeptide, preferably an IL-2Rβ intracellular domain, or (c) the first fusion protein comprises an intracellular portion of an IL-2Rβ polypeptide, preferably an IL-2Rβ intracellular domain, and the second fusion protein comprises an intracellular portion of an IL-2Rγ polypeptide, preferably an IL-2Rγ intracellular domain; and/or
(d) the first fusion protein comprises an IL-2Rβ transmembrane domain, an IL-2Rγ transmembrane domain, a TGFβRI transmembrane domain, or a TGFβR2 transmembrane domain, and the second fusion protein comprises an IL-2Rβ transmembrane domain, an IL-2Rγ transmembrane domain, a TGFβRI transmembrane domain, or a TGFβR2 transmembrane domain.

Embodiment 51. The host cell of Embodiment 49 or 50, wherein:
(i) the first fusion protein comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.:5, 10, 13, and 18; and
(ii) the second fusion protein comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 6, 9, 14, and 17.

Embodiment 52. The host cell of Embodiment 51, wherein the first fusion protein and the second protein comprise an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NOs: (i) 5 and 6, respectively; (ii) 5 and 9, respectively; (iii) 5 and 14, respectively; (iv) 5 and 17, respectively; (v) 10 and 6, respectively; (vi) 10 and 9, respectively; (vii) 10 and 14, respectively; (viii) 10 and 17, respectively; (ix) 13 and 6, respectively; (x) 13 and 9, respectively; (xi) 13 and 14, respectively; (xii) 13 and 17, respectively; (xiii) 18 and 6, respectively; (xiv) 18 and 9, respectively; (xv) 18 and 14, respectively; or (xvi) 18 and 17, respectively.

Embodiment 53. The host cell of any one of Embodiments 50-52, wherein the host cell encodes an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.:7, 11, 15, and 19.

Embodiment 54. The host cell of any one of Embodiments 50-53, wherein the host cell expresses at its cell surface a protein complex comprising the first fusion protein and the second fusion protein, wherein the protein complex is capable of binding to a TGFβ polypeptide, which is optionally comprised in a TGFβ dimer.

Embodiment 55. The host cell of any one of Embodiments 46-54, wherein the host cell comprises a hematopoietic progenitor cell or a human immune system cell.

Embodiment 56. The host cell of any one of Embodiments 46-55, wherein the host cell comprises a CD4+ T cell, a CD8+ T cell, a CD4− CD8− double negative T cell, a γδ T cell, a natural killer cell, a natural killer T cell, a dendritic cell, or any combination thereof.

Embodiment 57. The host cell of Embodiment any one of Embodiments 46-56, wherein the host cell comprises a T cell.

Embodiment 58. The host cell of Embodiment 57, wherein the T cell comprises a naïve T cell, a central memory T cell, a stem cell memory T cell, an effector memory T cell, or any combination thereof.

Embodiment 59. The host cell of any one of Embodiments 46-58, comprising a chromosomal gene knockout or a mutation of: a TGFβR1 gene locus, a TGFβR2 gene locus, a PD-1 gene locus, a CTLA4 gene locus, a LAT gene locus, a TIM-3 gene locus, a PD-L1 gene locus, a TIGIT gene locus, an A2AR gene locus, a Fas locus, a FasL gene locus, a B7-H3 gene locus, a B7-H4 gene locus, an IDO gene locus, a VISTA gene locus, a SIGLEC7 gene locus, a SIGLEC9 gene locus, a TRAC gene locus, a TRBC gene locus, a T cell receptor gene locus, a MHC (e.g. HLA) gene locus, a CBLB gene locus, a RASA2 gene locus, a UBASH3A gene locus, a CISH gene locus, or any combination thereof.

Embodiment 60. The host cell of any one of Embodiments 46-59, wherein the host cell is modified (e.g., having a chromosomal knockout mutation and/or a chromosomal missense mutation and/or a chromosomal splice junction mutation; encoding an inhibitory nucleic acid such as an siRNA or an antisense oligonucleotide) to have reduced protein expression (including null expression), of an endogenous TGFβR1, an endogenous TGFβR2, or both, as compared to the unmodified host cell.

Embodiment 61. The host cell of any one of Embodiments 46-60, further comprising a polynucleotide encoding an antigen-binding protein.

Embodiment 62. The host cell of any one of Embodiments 46-61, which expresses a/the antigen-binding protein.

Embodiment 63. The host cell of Embodiment 61 or 62, wherein the antigen-binding protein comprises a TCR (optionally wherein the TCR is endogenous to the host cell), an antigen-binding fragment of a TCR (e.g. a scTv), a scTCR, a CAR, or any combination thereof.

Embodiment 64. The host cell of any one of Embodiments 61-63, wherein the antigen-binding protein binds to: a cancer antigen; an autoimmune antigen; a viral antigen; a bacterial antigen; a fungal antigen; a parasitic antigen; or any combination thereof.

Embodiment 65. The host cell of Embodiment 64, wherein the cancer comprises a solid tumor, a hematological malignancy, or both.

Embodiment 66. The host cell of any one of Embodiments 64-65, wherein the antigen is selected from a WT1, mesothelin, KRAS, ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, NY-ESO, 5T4, BCMA, FAP, Carbonic anhydrase 9, BRAF, α-fetoprotein, MAGE-A3, MAGE-A4, SSX-2, PRAME, HA-1, p2M, ETA, tyrosinase, NRAS, or CEA antigen.

Embodiment 67. The fusion protein of any one of Embodiments 1-24, the polynucleotide of any one of Embodiments 25-39, the vector of any one of Embodiments 40-45, or the host cell of any one of Embodiments 46-66, wherein the TGFβ polypeptide comprises a TGFB1, a TGFB2, a TGFB3, or any combination thereof, optionally comprised in a TGFB polypeptide dimer.

Embodiment 68. The host cell of any one of Embodiments 46-67, wherein the host cell comprises an increased level of phosphorylated STAT5 when the host cell is in the presence of a TGFβ, as compared to the level of phosphorylated STAT5 when the host cell is not in the presence of the TGFβ.

Embodiment 69. The host cell of any one of Embodiments 46-67, wherein the host cell comprises an IL-2R signal when the host cell is in the presence of a TGFβ polypeptide (e.g., a TGFβ polypeptide dimer).

Embodiment 70. The host cell of any one of Embodiments 46-69, wherein the host cell performs one or more of the following when in the presence of a TGFβ polypeptide (e.g., a TGFβ polypeptide dimer): proliferation; growth; expression of an effector molecule (e.g., a T cell receptor); glycolytic metabolism; expression of protein (e.g., MYC, SLC7A5, or both); and biosynthesis of cholesterol.

Embodiment 71. The host cell of any one of Embodiments 46-70, wherein the host cell has a reduced level of phosphorylated SMAD2/SMAD3 when the host cell is in the presence of a TGFβ polypeptide (e.g., a TGFβ polypeptide dimer), as compared to a reference cell,
wherein the reference cell does not encode or express the fusion protein(s), and, optionally, is not modified to have reduced protein expression (including null expression), of an endogenous TGFβR1, an endogenous TGFβR2, or both, as compared to the reference cell without the modification.

Embodiment 72. The host cell of any one of Embodiments 46-71, wherein the host cell comprises an increased level of phosphorylated STAT5 when the host cell is in the presence of a TGFβ, as compared to the level of phosphorylated STAT5 comprised in a reference cell not encoding or expressing the fusion protein(s), when the reference cell is in the presence of the TGFβ.

Embodiment 73. The host cell of any one of Embodiments 46-72, wherein the host cell comprises an increased level of phosphorylated STAT5 when the host cell is in the presence of an IL-2, as compared to the level of phosphorylated STAT5 comprised in a reference cell that does not encode or express the fusion protein(s), respectively, when the reference cell is in the presence of the IL-2.

Embodiment 74. The host cell of any one of Embodiments 61-73, wherein the host cell, when in the presence of a TGFβ polypeptide (optionally comprised in a TGFβ polypeptide dimer), is capable of killing antigen-expressing cells at a level that is about the same as the level of killing by a reference cell when the reference cell is in the presence of an IL-2 polypeptide.

Embodiment 75. A composition comprising: (i) the fusion protein of any one of Embodiments 1-24 and 67; and/or (ii) the polynucleotide of any one of Embodiments 25-39 and 67; and/or (iii) the vector of any one of Embodiments 40-45 and 67; and/or (iv) the host cell of any one of Embodiments 46-74, and a pharmaceutically acceptable carrier, excipient, or diluent.

Embodiment 76. The composition of Embodiment 75, comprising (i) a composition comprising at least about 30% CD4+T host cells, combined with (ii) a composition comprising at least about 30% CD8+T host cells, in about a 1:1 ratio.

Embodiment 77. A method of treating a disease or condition in a subject, the method comprising administering to the subject an effective amount of: (i) the fusion protein of any one of Embodiments 1-24 and 67; and/or (ii) the polynucleotide of any one of Embodiments 25-39 and 67; and/or (iii) the vector of any one of Embodiments 40-45 and 67; and/or (iv) the host cell of any one of Embodiments 46-74; and/or (v) the composition of Embodiment 75 or 76.

Embodiment 78. The fusion protein of any one of Embodiments 1-24 and 67, and/or the polynucleotide of any one of Embodiments 25-39 and 67, and/or the vector of any one of Embodiments 40-45 and 67, and/or the host cell of any one of Embodiments 46-74, and/or the composition of Embodiment 75 or 76, for use in a method of treating a disease or condition in a subject.

Embodiment 79. The fusion protein of any one of Embodiments 1-24 and 67, and/or the polynucleotide of any one of Embodiments 25-39 and 67, and/or the vector of any one of Embodiments 40-45 and 67, and/or the host cell of any one of Embodiments 46-74, and/or the composition of Embodiment 75 or 76, for use in a the preparation of a medicament for treating a disease or condition in a subject.

Embodiment 80. The method of Embodiment 77 or the fusion protein, polynucleotide, vector, host cell, or composition for use of Embodiment 78 or 79, wherein the disease or condition is associated with or characterized by expression of an antigen, wherein the antigen is specifically bound by the antigen-binding protein.

Embodiment 81. The method of Embodiment 77 or 80, or the fusion protein, polynucleotide, vector, host cell, or composition for use of any one of Embodiments 78-80, wherein the disease or condition comprises or is a hyperproliferative disease, a proliferative disease, an autoimmune disease, a neurodegenerative disease, or an infection.

Embodiment 82. The method of Embodiment 77, 80, or 81 or the fusion protein, polynucleotide, vector, host cell, or composition for use of any one of Embodiments 78-81, wherein the disease or condition is a cancer.

Embodiment 83. The method of Embodiment 82 or the fusion protein, polynucleotide, vector, host cell, or composition for use of Embodiment 82, wherein the cancer comprises a carcinoma, a sarcoma, a glioma, a lymphoma, a leukemia, a myeloma, or any combination thereof.

Embodiment 84. The method of Embodiment 82 or 83, or the fusion protein, polynucleotide, vector, host cell, or composition for use of Embodiment 82 or 83, wherein the cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

Embodiment 85. The method of any one of Embodiments 82-84, or the fusion protein, polynucleotide, vector, host cell, or composition for use of any one of Embodiments 82-84, wherein the cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenstrom's macroglobulinemia, CD37⁺ dendritic cell lymphoma, lymphoplasmacytic lymphoma, splenic marginal zone lymphoma, extranodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

Embodiment 86. The method of Embodiment 82 or the fusion protein, polynucleotide, vector, host cell, or composition for use of Embodiment 82, wherein the cancer comprises a solid tumor.

Embodiment 87. The method of Embodiment 86 or the fusion protein, polynucleotide, vector, host cell, or composition for use of Embodiment 86, wherein the solid tumor is a sarcoma or a carcinoma.

Embodiment 88. The method of Embodiment 87 or the fusion protein, polynucleotide, vector, host cell, or composition for use of Embodiment 87, wherein the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

Embodiment 89. The method of any one of Embodiments 86-88 or the fusion protein, polynucleotide, vector, host cell, or composition for use of any one of Embodiments 86-88, wherein the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypemephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma).

Embodiment 90. The method of any one of Embodiments 86-89, or the fusion protein, polynucleotide, vector, host cell, or composition for use of any one of Embodiments 86-89, wherein the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

Embodiment 91. The method of any one of Embodiments 77 and 80-90, or the fusion protein, polynucleotide, vector, host cell, or composition for use of any one of Embodiments 78-90, wherein the host cell is an allogeneic cell, a syngeneic cell, or an autologous cell.

Embodiment 92. The method of any one of Embodiments 77 and 80-91, or the fusion protein, polynucleotide, vector, host cell, or composition for use of any one of Embodiments 78-91, wherein the method comprises administering a plurality of doses of the fusion protein, polynucleotide, vector, host cell, or composition to the subject.

Embodiment 93. The method of Embodiment 92 or the fusion protein, polynucleotide, vector, host cell, or composition for use of Embodiment 92, wherein the plurality of doses are administered at intervals between administrations of about two, three, four, five, six, seven, eight, or more weeks.

Embodiment 94. The method of Embodiment 92 or 93, or the fusion protein, polynucleotide, vector, host cell, or composition for use of Embodiment 92 or 93, wherein a dose of the host cell comprises about 10⁵ cells/m² to about 10¹¹ cells/m².

Embodiment 95. The method of any one of Embodiments 77 and 80-94, or the fusion protein, polynucleotide, vector, host cell, or composition for use of any one of Embodiments 78-94, wherein the subject is receiving, has received, or will receive one or more of: (i) chemotherapy; (ii) radiation therapy; (iii) an inhibitor of an immune suppression component; (iv) an agonist of a stimulatory immune checkpoint agent; (v) RNAi; (vi) a cytokine; (vii) a surgery; (viii) a monoclonal antibody and/or an antibody-drug conjugate; or (ix) any combination of (i)-(viii), in any order.

Embodiment 1a. A fusion protein comprising: (i) an extracellular component comprising an extracellular domain of a transforming growth factor beta receptor (TGFβR) polypeptide, or a portion or variant thereof that is capable of binding to a TGFβ polypeptide; (ii) an intracellular component comprising an intracellular portion of an interleukin-2 receptor (IL-2R) polypeptide; and (iii) a transmembrane component disposed between the extracellular component of (i) and the intracellular component of (ii), wherein the fusion protein, when expressed by a host cell and bound to the TGFβ polypeptide, is capable of contributing to an IL-2 signal in the host cell.

Embodiment 2a. The fusion protein of Embodiment Ia, wherein the TGFβR polypeptide comprises a TGFβRI polypeptide or a TGFβR2 polypeptide.

Embodiment 3a. The fusion protein of Embodiment 1a or 2a, wherein the IL-2R polypeptide comprises an IL-2Rβ polypeptide, an IL-2Rγ polypeptide, or both.

Embodiment 4a. A fusion protein comprising: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor beta (IL-2Rβ) polypeptide; and (iii) a transmembrane component disposed between the extracellular component of (i) with the intracellular component of (ii), wherein the fusion protein, when expressed by a host cell and bound to the TGFβ polypeptide, is capable of contributing to an IL-2 signal in the host cell.

Embodiment 5a. A fusion protein comprising: (i) an extracellular component comprising an extracellular domain of a transforming growth factor beta-receptor 2 (TGFβR2) polypeptide, or a portion or variant thereof that is capable of binding to a TGFβ polypeptide; (ii) an intracellular component comprising an intracellular portion of an interleukin-2 receptor beta (IL-2Rβ) polypeptide; and (iii) a transmembrane component disposed between the extracellular component of (i) with the intracellular component of (ii), wherein the fusion protein, when expressed by a host cell and bound to the TGFβ polypeptide, is capable of contributing to an IL-2 signal in the host cell.

Embodiment 6a. A fusion protein comprising: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor gamma (IL-2Rγ) polypeptide; and (iii) a transmembrane component disposed between the extracellular component of (i) with the intracellular component of (ii), wherein the fusion protein, when expressed by a host cell and bound to the TGFβ polypeptide, is capable of contributing to an IL-2 signal in the host cell.

Embodiment 7a. A fusion protein comprising: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor gamma (IL-2Rγ) polypeptide; and (iii) a transmembrane component disposed between the extracellular component of (i) with the intracellular component of (ii), wherein the fusion protein, when expressed by a host cell and bound to the TGFβ polypeptide, is capable of contributing to an IL-2 signal in the host cell.

Embodiment 8a. The fusion protein of any one of Embodiments 1a-7a, wherein the transmembrane component comprises a transmembrane domain from IL-2Rβ.

Embodiment 9a. The fusion protein of any one of Embodiments 1a-8a, wherein the transmembrane component comprises a transmembrane domain from IL-2Rγ.

Embodiment 10a. The fusion protein of any one of Embodiments 1a-9a, wherein the transmembrane component comprises a transmembrane domain from TGFβRI and wherein, optionally, the intracellular component of the fusion protein further comprises, disposed N-terminal to the IL-2R intracellular portion, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal amino acid(s) from TGFβR1 intracellular domain, or a variant thereof comprising one, two, three, four, or five, optionally conservative, amino acid substitutions, wherein, further optionally, the intracellular component of the fusion protein comprises the amino acids C-H-N disposed N-terminal to the IL-2R intracellular portion.

Embodiment 11a. The fusion protein of any one of Embodiments 1a-10a, wherein the transmembrane component comprises a transmembrane domain from TGFβR2 and wherein, optionally, the intracellular component of the fusion protein further comprises, disposed N-terminal to the IL-2R intracellular portion, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal amino acid(s) from TGFβR2 intracellular domain, or a variant thereof comprising one, two, three, four, or five, optionally conservative, amino acid substitutions, wherein, further optionally, the intracellular component of the fusion protein comprises the amino acids R-V-N disposed N-terminal to the IL-2R intracellular portion.

Embodiment 12a. A fusion protein comprising: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor beta (IL-2Rβ) polypeptide, wherein the intracellular component optionally comprises an IL-2Rβ intracellular domain; and (iii) a transmembrane component disposed between the extracellular component of (i) with the intracellular component of (ii), wherein the transmembrane component optionally comprises an IL-2Rβ transmembrane domain, wherein the fusion protein, when expressed by a host cell and bound to the TGFβ polypeptide, is capable of contributing to an IL-2 signal in the host cell.

Embodiment 13a. A fusion protein comprising: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor beta (IL-2Rβ) polypeptide, wherein the intracellular component optionally comprises an IL-2Rβ intracellular domain; and (iii) a transmembrane component disposed between the extracellular component of (i) with the intracellular component of (ii), wherein the transmembrane component optionally comprises an IL-2Rβ transmembrane domain, wherein the fusion protein, when expressed by a host cell and bound to the TGFβ polypeptide, is capable of contributing to an IL-2 signal in the host cell.

Embodiment 14a. A fusion protein comprising: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor beta (IL-2Rβ) polypeptide, wherein the intracellular component optionally comprises a IL-2Rβ intracellular domain; and (iii) a transmembrane component disposed between the extracellular component of (i) with the intracellular component of (ii), wherein the transmembrane component optionally comprises a TGFβR1 transmembrane domain, wherein the fusion protein, when expressed by a host cell and bound to the TGFβ polypeptide, is capable of contributing to an IL-2 signal in the host cell.

Embodiment 15a. A fusion protein comprising: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor beta (IL-2Rβ) polypeptide, wherein the intracellular component optionally comprises a IL-2Rβ intracellular domain; and (iii) a transmembrane component disposed between the extracellular component of (i) with the intracellular component of (ii), wherein the transmembrane component optionally comprises a TGFβR2 transmembrane domain, wherein the fusion protein, when expressed by a host cell and bound to the TGFβ polypeptide, is capable of contributing to an IL-2 signal in the host cell.

Embodiment 16a. A fusion protein comprising: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor gamma (IL-2Rγ) polypeptide, wherein the intracellular component optionally comprises a IL-2Rγ intracellular domain; and (iii) a transmembrane component disposed between the extracellular component of (i) with the intracellular component of (ii), wherein the transmembrane component optionally comprises an IL-2Rγ transmembrane domain, wherein the fusion protein, when expressed by a host cell and bound to the TGFβ polypeptide, is capable of contributing to an IL-2 signal in the host cell.

Embodiment 17a. A fusion protein comprising: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor gamma (IL-2Rγ) polypeptide, wherein the intracellular component optionally comprises a IL-2Rγ intracellular domain; and (iii) a transmembrane component disposed between the extracellular component of (i) with the intracellular component of (ii), wherein the transmembrane component optionally comprises an IL-2Rγ transmembrane domain, wherein the fusion protein, when expressed by a host cell and bound to the TGFβ polypeptide, is capable of contributing to an IL-2 signal in the host cell.

Embodiment 18a. A fusion protein comprising: (i) an extracellular component comprising an extracellular domain of a transforming growth factor beta receptor 2 (TGFβR2) polypeptide, or a portion or variant thereof that is capable of binding to a TGFβ polypeptide; (ii) an intracellular component comprising an intracellular portion of an interleukin-2 receptor gamma (IL-2Rγ) polypeptide, wherein the intracellular component optionally comprises a IL-2Rγ intracellular domain; and (iii) a transmembrane component disposed between the extracellular component of (i) with the intracellular component of (ii), wherein the transmembrane component optionally comprises an TGFβR2 transmembrane domain, wherein the fusion protein, when expressed by a host cell and bound to the TGFβ polypeptide, is capable of contributing to an IL-2 signal in the host cell.

Embodiment 19a. A fusion protein comprising: (i) an extracellular component comprising an extracellular domain of a transforming growth factor beta receptor 1 (TGFβR1) polypeptide, or a portion or variant thereof that is capable of binding to a TGFβ polypeptide; (ii) an intracellular component comprising an intracellular portion of an interleukin-2 receptor gamma (IL-2Rγ) polypeptide, wherein the intracellular component optionally comprises a IL-2Rγ intracellular domain; and (iii) a transmembrane component disposed between the extracellular component of (i) with the intracellular component of (ii), wherein the transmembrane component optionally comprises an TGFβR1 transmembrane domain, wherein the fusion protein, when expressed by a host cell and bound to the TGFβ polypeptide, is capable of contributing to an IL-2 signal in the host cell.

Embodiment 20a. The fusion protein of any one of Embodiments 1a-19a, wherein: (1) (i) the extracellular component has at least 90%, at least 91%, at least 92%, at least 93%, at least 94%, at least 95%, at least 96%, at least 97%, at least 98%, or at least 99% identity to, or comprises or consists of, the amino acid sequence set forth in SEQ ID NO.:39 or 41; (ii) the transmembrane component has at least 90%, at least 91%, at least 92%, at least 93%, at least 94%, at least 95%, at least 96%, at least 97%, at least 98%, or at least 99% identity to, or comprises or consists of, the amino acid sequence set forth in: SEQ ID NO.:40; SEQ ID NO.:42; SEQ ID NO.:43; or SEQ ID NO.:45; and (iii) the intracellular component has at least 90%, at least 91%, at least 92%, at least 93%, at least 94%, at least 95%, at least 96%, at least 97%, at least 98%, or at least 99% identity to, or comprises or consists of, the amino acid sequence set forth in SEQ ID NO.:44 or 46, and wherein, optionally, the fusion protein comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOS.:5, 6, 9, 10, 13, 14, 17, and 18; and/or (2) wherein the fusion protein comprises one or more amino acid substitutions, insertions, and/or deletions to reduce or prevent the fusion protein from forming a protein dimer with a human TGFβRI or a human TGFβR2, wherein, optionally, the one or more amino acid substitutions, insertions, and/or deletions provide: (i) a variant of SEQ ID NO.:47, wherein the variant comprises from one to ten amino acid insertions, or from one to ten amino acid deletions; (ii) one, two, three, four, five, six, seven, eight, nine, ten, or more amino acids added to the N-terminus and/or to the C-terminus of SEQ ID NO.:47; (iii) a variant of SEQ ID NO.:48, wherein the variant comprises from one to ten amino acid insertions, or from one to ten amino acid deletions; (iv) one, two, three, four, five, six, seven, eight, nine, ten, or more added to the N-terminus and/or to the C-terminus of SEQ ID NO.:48.

Embodiment 21a. The fusion protein of any one of Embodiments 1a-20a, wherein the transmembrane component comprises a transmembrane domain from the TGFβR polypeptide and the intracellular component comprises, extending from the transmembrane domain and N-terminal to the intracellular portion of the IL-2R polypeptide, a TGFβR intracellular overhang or intracellular overhang sequence e.g. as described for Table A, wherein, optionally: (i) the transmembrane component comprises a TGFβR1 transmembrane domain and the intracellular component comprises, extending from the TGFβR1 transmembrane domain and N-terminal to the intracellular portion of the IL-2R polypeptide, a TGFβR1 intracellular overhang or intracellular overhang sequence e.g. as described for Table A, optionally comprising, consisting essentially of, or consisting of the amino acid sequence C-H-N; or (ii) the transmembrane component comprises a TGFβR2 transmembrane domain and the intracellular component comprises, extending from the TGFβR2 transmembrane domain and N-terminal to the intracellular portion of the IL-2R polypeptide, a TGFβR2 intracellular overhang or intracellular overhang sequence e.g. as described for Table A, optionally comprising, consisting essentially of, or consisting of the amino acid sequence R-V-N.

Embodiment 22a. The fusion protein of any one of Embodiments 1a-21a, wherein the transmembrane component and a portion of the intracellular component together comprise the amino acid sequence of SEQ ID NO.:56 or SEQ ID NO.:60.

Embodiment 23a. The fusion protein of any one Embodiments 1a-22a, wherein the IL-2 signal comprises, results in, provides, or promotes any one or more of (i)-(vii): (i) phosphorylation of the intracellular portion of the IL-2R polypeptide by JAK1 or JAK3; (ii) phosphorylation in the host cell of any one or more of: PI3K; Akt; STAT; STAT5A; STAT5B; MEK; SHC1; MEK1; MEK2; ERK1; ERK2; and STAT3; (iii) STAT5-mediated transcription; (iv) recruitment of GRB2 and SOS; (v) GTP loading of RAS; (vi) activation of the Raf-ERK MAPK cascade; (vii) activation of mTORC1 and/or HIF1α/HIF1β.

Embodiment 24a. The fusion protein of any one Embodiments 1a-23a, wherein, when the fusion protein is expressed by a host cell and the fusion protein binds to the TGFβ polypeptide, the host cell performs one or more of the following: proliferation; growth: expression of an effector molecule (e.g., a T cell receptor); glycolytic metabolism; expression of protein (e.g., MYC, SLC7A5, or both); and biosynthesis of cholesterol.

Embodiment 25a. A fusion protein comprising: (i) an extracellular component comprising an extracellular domain from human TGFβR1; (ii) a transmembrane component comprising a transmembrane domain from human TGFβRI; and (iii) an intracellular component comprising a cytoplasmic/signaling domain from human IL-2Rβ, wherein, optionally, the intracellular component further comprises a human TGFβR1 intracellular overhang or intracellular overhang sequence e.g. as described for Table A, optionally comprising, consisting essentially of, or consisting of the amino acid sequence C-H-N, disposed N-terminal to the human IL-2Rβ cytoplasmic/signaling domain.

Embodiment 26a. A fusion protein comprising: (i) an extracellular component comprising an extracellular domain from human TGFβR1; (ii) a transmembrane component comprising a transmembrane domain from human TGFβRI; and (iii) an intracellular component comprising a cytoplasmic/signaling domain from human IL-2Rγ, wherein, optionally, the intracellular component further comprises a human TGFβR1 intracellular overhang or intracellular overhang sequence e.g. as described for Table A, optionally comprising, consisting essentially of, or consisting of the amino acid sequence C-H-N, disposed N-terminal to the human IL-2Rγ cytoplasmic/signaling domain.

Embodiment 27a. A fusion protein comprising: (i) an extracellular component comprising an extracellular domain from human TGFβR1; (ii) a transmembrane component comprising a transmembrane domain from human IL-2Rβ and (iii) an intracellular component comprising a cytoplasmic/signaling domain from human IL-2Rβ.

Embodiment 28a. A fusion protein comprising: (i) an extracellular component comprising an extracellular domain from human TGFβR1; (ii) a transmembrane component comprising a transmembrane domain from human IL-2Rγ; and (iii) an intracellular component comprising a cytoplasmic/signaling domain from IL-2Rγ.

Embodiment 29a. A fusion protein comprising: (i) an extracellular component comprising an extracellular domain from human TGFβRII; (ii) a transmembrane component comprising a transmembrane domain from human TGFβRII; and (iii) an intracellular component comprising a cytoplasmic/signaling domain from human IL-2Rβ, wherein, optionally, the intracellular component further comprises a human TGFβRII intracellular overhang or intracellular overhang sequence e.g. as described for Table A, optionally comprising, consisting essentially of, or consisting of the amino acid sequence R-V-N, disposed N-terminal to the IL-2Rβ cytoplasmic/signaling domain.

Embodiment 30a. A fusion protein comprising: (i) an extracellular component comprising an extracellular domain from human TGFβRII; (ii) a transmembrane component comprising a transmembrane domain from human TGFβRU; and (iii) an intracellular component comprising a cytoplasmic/signaling domain from human IL-2Rγ, wherein, optionally, the intracellular component further comprises a human TGFβRII intracellular overhang or intracellular overhang sequence e.g. as described for Table A, optionally comprising, consisting essentially of, or consisting of the amino acid sequence R-V-N, disposed N-terminal to the IL-2Rγ cytoplasmic/signaling domain.

Embodiment 31a. A fusion protein comprising: (i) an extracellular component comprising an extracellular domain from human TGFβRII; (ii) a transmembrane component comprising a transmembrane domain from human IL-2Rβ and (iii) an intracellular component comprising a cytoplasmic/signaling domain from human IL-2Rβ.

Embodiment 32a. A fusion protein comprising: (i) an extracellular component comprising an extracellular domain from human TGFβRII; (ii) a transmembrane component comprising a transmembrane domain from human IL-2Rγ; and (iii) an intracellular component comprising a cytoplasmic/signaling domain from IL-2Rγ.

Embodiment 33a. A fusion protein comprising or consisting of the amino acid sequence set forth in any one of SEQ ID NOs.:5, 6, 9, 10, 13, 14, 17, and 18.

Embodiment 34a. A fusion protein comprising or consisting of the amino acid sequence set forth in any one of SEQ ID NOs.:7, 11, 15, and 19.

Embodiment 35a. A polynucleotide encoding the fusion protein of any one of Embodiments 1a-34a.

Embodiment 36a. A polynucleotide encoding the fusion protein of any one of Embodiments 1a-34a, wherein the/an encoded fusion protein further comprises, N-terminal to and connected to the extracellular component, a signal peptide.

Embodiment 37a. The polynucleotide of Embodiment 36a, wherein the signal peptide comprises or consists of the amino acid sequence set forth in SEQ ID NO.:21 or 22.

Embodiment 38a. A polynucleotide encoding (1) a first fusion protein of any one of Embodiments 1-34a and (2) a second fusion protein of any one of Embodiments 1-34a, optionally wherein the first fusion protein and the second fusion protein comprise an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NOs: (i) 5 and 6, respectively; (ii) 5 and 9, respectively; (iii) 5 and 14, respectively; (iv) 5 and 17, respectively; (v) 10 and 6, respectively; (vi) 10 and 9, respectively; (vii) 10 and 14, respectively; (viii) 10 and 17, respectively; (ix) 13 and 6, respectively; (x) 13 and 9, respectively; (xi) 13 and 14, respectively; (xii) 13 and 17, respectively; (xiii) 18 and 6, respectively; (xiv) 18 and 9, respectively; (xv) 18 and 14, respectively; or (xvi) 18 and 17, respectively.

Embodiment 39a. The polynucleotide of Embodiment 38a, wherein an amino acid sequence of the first fusion protein is different than an amino acid sequence of the second fusion protein, wherein, optionally, the first and second fusion protein are according to Table B, and/or: (a) the first fusion protein comprises an extracellular domain of a TGFβR1 polypeptide, or a portion or variant thereof that is capable of binding to a TGFβ polypeptide, and the second fusion protein comprises an extracellular domain of a TGFβR2 polypeptide, or a portion or variant thereof that is capable of binding to a TGFβ polypeptide; and/or (b) the first fusion protein comprises an intracellular portion of an IL-2Rγ polypeptide, preferably an IL-2Rγ intracellular domain, and the second fusion protein comprises an intracellular portion of an IL-2Rβ polypeptide, preferably an IL-2Rβ intracellular domain, or (c) the first fusion protein comprises an intracellular portion of an IL-2Rβ polypeptide, preferably an IL-2R1p intracellular domain, and the second fusion protein comprises an intracellular portion of an IL-2Rγ polypeptide, preferably an IL-2Rγ intracellular domain; and/or (d) the first fusion protein comprises an IL-2Rβ transmembrane domain, an IL-2Rγ transmembrane domain, a TGFβRI transmembrane domain, or a TGFβR2 transmembrane domain, and the second fusion protein comprises an IL-2Rβ transmembrane domain, an IL-2Rγ transmembrane domain, a TGFβRI transmembrane domain, or a TGFβR2 transmembrane domain.

Embodiment 40a. The polynucleotide of Embodiment 39a, wherein: (i) the first fusion protein comprises an extracellular component comprising an extracellular domain of a transforming growth factor beta receptor 1 (TGFβR1) polypeptide, or a portion or variant thereof that is capable of binding to a TGFβ polypeptide, and the second fusion protein comprises an extracellular component comprising an extracellular domain of a transforming growth factor beta receptor 2 (TGFβR2) polypeptide, or a portion or variant thereof that is capable of binding to a TGFβ polypeptide; or (ii) the first fusion protein comprises an extracellular component comprising an extracellular domain of a transforming growth factor beta receptor 2 (TGFβR2) polypeptide, or a portion or variant thereof that is capable of binding to a TGFβ polypeptide, and the second fusion protein comprises an extracellular component comprising an extracellular domain of a transforming growth factor beta receptor 1 (TGFβR1) polypeptide, or a portion or variant thereof that is capable of binding to a TGFβ polypeptide.

Embodiment 41a. A polynucleotide encoding: (1) a first fusion protein comprising (1a) an extracellular component comprising an extracellular domain from (e.g. human) TGFβR1, (1b) a transmembrane component comprising a transmembrane domain from (e.g. human) IL-2Rβ, and (1c) an intracellular component comprising a cytoplasmic/signaling domain from (e.g. human) IL-2Rβ; and (2) a second fusion protein comprising (2a) an extracellular component comprising an extracellular domain from (e.g. human) TGFβR2, (2b) a transmembrane component comprising a transmembrane domain from (e.g. human) IL-2Rγ, and (2c) an intracellular component comprising a cytoplasmic/signaling domain from (e.g. human) IL-2Rγ,
wherein, optionally, polynucleotide further encodes an antigen-binding protein such as a TCR or a CAR.

Embodiment 42a. A polynucleotide encoding: (1) a first fusion protein comprising (1a) an extracellular component comprising an extracellular domain from (e.g. human) TGFβR2, (1b) a transmembrane component comprising a transmembrane domain from (e.g. human) TGFβR2, and (1c) an intracellular component comprising a cytoplasmic/signaling domain from (e.g. human) IL-2Rβ; and (2) a second fusion protein comprising (2a) an extracellular component comprising an extracellular domain from (e.g. human) TGFβR1, (2b) a transmembrane component comprising a transmembrane domain from (e.g. human) TGFβR1, and (2c) an intracellular component comprising a cytoplasmic/signaling domain from (e.g. human) IL-2Rγ, optionally wherein (i) the intracellular component further comprises the amino acids C-H-N disposed N-terminal to the IL-2Rγ cytoplasmic/signaling domain and/or (ii) the polynucleotide further encodes an antigen-binding protein such as a TCR or a CAR.

Embodiment 43a. The polynucleotide of any one of Embodiments 38a-42a, further comprising, disposed between a nucleotide sequence encoding the first fusion protein and a nucleotide sequence encoding the second fusion protein: (i) a nucleotide sequence encoding a furin cleavage site; (ii) a nucleotide sequence encoding a self-cleaving polypeptide (e.g., P2A, T2A, E2A, F2A); or (iii) both (i) and (ii), wherein, optionally, (i) is disposed 5′ to (i.e. upstream of) (ii).

Embodiment 44a. The polynucleotide of Embodiment 43a, comprising, in 5′ to 3′ direction: a nucleotide sequence encoding the first fusion protein; a nucleotide sequence encoding a furin cleavage site; a nucleotide encoding a self-cleaving polypeptide with an optional N-terminal linker (e.g., G-S-G); and a nucleotide sequence encoding the second fusion protein.

Embodiment 45a. The polynucleotide of Embodiment 44a, wherein:
(i) the first fusion protein comprises (1a) an extracellular component comprising an extracellular domain from TGFβR1, or a portion or variant thereof that is capable of binding to a TGFβ polypeptide, (1b) a transmembrane component comprising a transmembrane domain from IL-2Rβ, and (1c) an intracellular component comprising an intracellular domain from IL-2Rβ, and the second fusion protein comprises (2a) an extracellular component comprising an extracellular domain from TGFβR2, or a portion or variant thereof that is capable of binding to a TGFβ polypeptide, (2b) a transmembrane component comprising a transmembrane domain from IL-2Rγ, and (2c) an intracellular component comprising an intracellular domain from IL-2Rγ; (ii) the first fusion protein comprises (1a) an extracellular component comprising an extracellular domain from TGFβR2, or a portion or variant thereof that is capable of binding to a TGFβ polypeptide, (1b) a transmembrane component comprising a transmembrane domain from IL-2Rβ, and (1c) an intracellular component comprising an intracellular domain from IL-2Rβ, and the second fusion protein comprises (2a) an extracellular component comprising an extracellular domain from TGFβR1, or a portion or variant thereof that is capable of binding to a TGFβ polypeptide, (2b) a transmembrane component comprising a transmembrane domain from IL-2Rγ, and (2c) an intracellular component comprising an intracellular domain from IL-2Rγ; (iii) the first fusion protein comprises (1a) an extracellular component comprising an extracellular domain from TGFβR1, or a portion or variant thereof that is capable of binding to a TGFβ polypeptide, (1b) a transmembrane component comprising a transmembrane domain from TGFβR1, and (1c) an intracellular component comprising (1)(c)(1) an optional TGFβRI intracellular overhang or overhang sequence, e.g. as described for Table A and optionally comprising, consisting essentially or, or consisting of the amino acids C-H-N, and (1)(c)(2) an intracellular domain from IL-2Rβ, and the second fusion protein comprises (2a) an extracellular component comprising an extracellular domain from TGFβR2, or a portion or variant thereof that is capable of binding to a TGFβ polypeptide, (2b) a transmembrane component comprising a transmembrane domain from TGFβR2, and (2c) an intracellular component comprising (2)(c)(1) an optional TGFβR2 intracellular overhang or overhang sequence, e.g. as described for Table A and optionally comprising, consisting essentially or, or consisting of the amino acids R-V-N, and (2)(c)(2) an intracellular domain from IL-2Rγ; or (iv) the first fusion protein comprises (1a) an extracellular component comprising an extracellular domain from TGFβR2, or a portion or variant thereof that is capable of binding to a TGFβ polypeptide, (1b) a transmembrane component comprising a transmembrane domain from TGFβR2, and (1c) an intracellular component comprising (1)(c)(1) an optional TGFβR2 intracellular overhang or overhang sequence, e.g. as described for Table A and optionally comprising, consisting essentially or, or consisting of the amino acids R-V-N and (1)(c)(2) an intracellular domain from IL-2Rβ, and the second fusion protein comprises (2a) an extracellular component comprising an extracellular domain from TGFβR1, or a portion or variant thereof that is capable of binding to a TGFβ polypeptide, (2b) a transmembrane component comprising a transmembrane domain from TGFβR1, and (2c) an intracellular component comprising (2)(c)(1) an optional an optional TGFβR1 intracellular overhang or overhang sequence, e.g. as described for Table A and optionally comprising, consisting essentially or, or consisting of the amino acids C-H-N and (2)(c)(2) an intracellular domain from IL-2Rγ.

Embodiment 46a. The polynucleotide of any one of Embodiments 38a-45a, which is codon optimized for expression in a host cell, wherein the host cell is optionally a human host cell, further optionally a human immune system cell, still further optionally a human T cell (e.g. a CD4+ T cell, a CD8+ T cell, a CD4−CD8− double negative T cell, a γδ T cell, or any combination thereof), a human NK cell, or a human NK-T cell.

Embodiment 47a. The polynucleotide of Embodiment 46a, which is codon optimized for expression in a T cell (e.g. a CD4+ T cell, a CD8+ T cell, a CD4−CD8− double negative T cell, a γδ T cell, or any combination thereof), a NK cell, or a NK-T cell, wherein the cell is preferably human.

Embodiment 48a. The polynucleotide of any one of Embodiments 38a-47a, wherein the polynucleotide has at least 50%, at least 55%, at least 60%, at least 65%, at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identity to, or comprises or consists of, the amino acid sequence set forth in any one of SEQ ID NOs.:8, 12, 16, and 20.

Embodiment 49a. The polynucleotide of any one of Embodiments 38a-48a, further comprising a nucleotide sequence that encodes an antigen-binding protein.

Embodiment 50a. The polynucleotide of Embodiment 49a, wherein the antigen-binding protein comprises a TCR, an antigen-binding fragment of a TCR (e.g. a scTv), a scTCR, a CAR, or any combination thereof.

Embodiment 51a. The polynucleotide of Embodiment 50a, wherein the antigen-binding protein comprises an αβ TCR, and wherein the polynucleotide comprises, in 5′ to 3′ direction: (1) a nucleotide sequence encoding the TCRβ chain; a nucleotide sequence encoding a furin cleavage site; a nucleotide sequence encoding a self-cleaving polypeptide with an optional N-terminal linker (e.g, G-S-G); a nucleotide sequence encoding the first fusion protein; a nucleotide sequence encoding a furin cleavage site; a nucleotide encoding a self-cleaving polypeptide with an optional N-terminal linker (e.g., G-S-G); a nucleotide sequence encoding the second fusion protein; a nucleotide sequence encoding a furin cleavage site; a nucleotide sequence encoding a self-cleaving polypeptide with an optional N-terminal linker (e.g, G-S-G); and a nucleotide sequence encoding the TCRα chain; or (2) a nucleotide sequence encoding the TCRα chain; a nucleotide sequence encoding a furin cleavage site; a nucleotide sequence encoding a self-cleaving polypeptide with an optional N-terminal linker (e.g, G-S-G); a nucleotide sequence encoding the first fusion protein; a nucleotide sequence encoding a furin cleavage site; a nucleotide encoding a self-cleaving polypeptide with an optional N-terminal linker (e.g., G-S-G); a nucleotide sequence encoding the second fusion protein; a nucleotide sequence encoding a furin cleavage site; a nucleotide sequence encoding a self-cleaving polypeptide with an optional N-terminal linker (e.g, G-S-G); and a nucleotide sequence encoding the TCRβ chain.

Embodiment 52a. The polynucleotide of any one of Embodiments 35a-51a, further comprising a nucleotide sequence encoding a transduction marker.

Embodiment 53a. The polynucleotide of Embodiment 52a, wherein the encoded transduction marker comprises EGFRt, CD19t, CD34t, or NGFRt.

Embodiment 54a. The polynucleotide of any one of Embodiments 35a-53a, further comprising a nucleotide sequence encoding a guide RNA, where, optionally, the guide RNA is specific for a TGFβRI gene locus, a TGFβR2 gene locus, a PD-1 gene locus, a CTLA4 gene locus, a LAT gene locus, a TIM-3 gene locus, a PD-L1 gene locus, a TIGIT gene locus, an A2AR gene locus, a Fas locus, a FasL gene locus, a B7-H3 gene locus, a B7-H4 gene locus, an IDO gene locus, a VISTA gene locus, a SIGLEC7 gene locus, a SIGLEC9 gene locus, a TRAC gene locus, a TRBC gene locus, a T cell receptor gene locus, a MHC (e.g. HLA) gene locus, a CBLB gene locus, a RASA2 gene locus, a UBASH3A gene locus, a CISH gene locus, or any combination thereof.

Embodiment 55a. The polynucleotide of any one of Embodiments 35a-54a, further comprising a nucleotide sequence encoding a Cas nuclease, wherein, optionally, the Cas nuclease comprises a Cas9 or a functional variant thereof, a Cas12 or a functional variant thereof, a Cas13 or a functional variant thereof, or a Cas14 or a functional variant thereof.

Embodiment 56a. A vector comprising the polynucleotide of any one of Embodiments 35a-55a, optionally wherein the polynucleotide is operably linked to an expression control sequence.

Embodiment 57a. The vector of Embodiment 56a, wherein the vector is capable of delivering the polynucleotide to a hematopoietic progenitor cell or a human immune system cell.

Embodiment 58a. The vector of Embodiment 57a, wherein the human immune system cell comprises a T cell, such as a CD4+ T cell, a CD8+ T cell, a CD4−CD8− double negative T cell, or a γδ T cell, a natural killer cell, a natural killer T cell, a dendritic cell, or any combination thereof.

Embodiment 59a. The vector of Embodiment 58a, wherein the T cell comprises a naïve T cell, a central memory T cell, a stem cell memory T cell, an effector memory T cell, or any combination thereof.

Embodiment 60a. The vector of any one of Embodiments 56a-59a, wherein the vector is a viral vector.

Embodiment 61a. The vector of Embodiment 60a, wherein the viral vector is a lentiviral vector or a γ-retroviral vector.

Embodiment 62a. A host cell expressing the fusion protein of any one of Embodiments 1a-34a.

Embodiment 63a. A host cell comprising the polynucleotide of any one of Embodiments 35a-55a.

Embodiment 64a. A host cell comprising the vector of any one of Embodiments 56a-61a.

Embodiment 65a. The host cell of any one of Embodiments 62a-64a, expressing or encoding (1) a first fusion protein of any one of Embodiments 1a-34a and (2) a second fusion protein of any one of Embodiments 1a-34a.

Embodiment 66a. The host cell of Embodiment 65a, wherein an amino acid sequence of the first fusion protein is different than an amino acid sequence of the second fusion protein, wherein, optionally, the first and second fusion protein are according to Table B, and/or:
(a) the first fusion protein comprises an extracellular domain of a TGFβRI polypeptide, or a portion or variant thereof that is capable of binding to a TGFβ polypeptide, and the second fusion protein comprises an extracellular domain of a TGFβR2 polypeptide, or a portion or variant thereof that is capable of binding to a TGFβ polypeptide; and/or
(b) the first fusion protein comprises an intracellular portion of an IL-2Rγ polypeptide, preferably an IL-2Rγ intracellular domain, and the second fusion protein comprises an intracellular portion of an IL-2Rβ polypeptide, preferably an IL-2R$ intracellular domain, or (c) the first fusion protein comprises an intracellular portion of an IL-2Rβ polypeptide, preferably an IL-2Rβ intracellular domain, and the second fusion protein comprises an intracellular portion of an IL-2Rγ polypeptide, preferably an IL-2Rγ intracellular domain; and/or
(d) the first fusion protein comprises an IL-2Rβ transmembrane domain, an IL-2Rγ transmembrane domain, a TGFβRI transmembrane domain, or a TGFβR2 transmembrane domain, and the second fusion protein comprises an IL-2Rβ transmembrane domain, an IL-2Rγ transmembrane domain, a TGFβRI transmembrane domain, or a TGFβR2 transmembrane domain.

Embodiment 67a. The host cell of Embodiment 66a, wherein:
(i) the first fusion protein comprises an extracellular component comprising an extracellular domain of a transforming growth factor beta receptor 1 (TGFβR1) polypeptide, or a portion or variant thereof that is capable of binding to a TGFβ polypeptide, and the second fusion protein comprises an extracellular component comprising an extracellular domain of a transforming growth factor beta receptor 2 (TGFβR2) polypeptide, or a portion or variant thereof that is capable of binding to a TGFβ polypeptide; or
(ii) the first fusion protein comprises an extracellular component comprising an extracellular domain of a transforming growth factor beta receptor 2 (TGFβR2) polypeptide, or a portion or variant thereof that is capable of binding to a TGFβ polypeptide, and the second fusion protein comprises an extracellular component comprising an extracellular domain of a transforming growth factor beta receptor 1 (TGFβR1) polypeptide, or a portion or variant thereof that is capable of binding to a TGFβ polypeptide.

Embodiment 68a. The host cell of any one of Embodiments 65a-67a, wherein: (1) the first fusion protein comprises (1a) an extracellular component comprising an extracellular domain from (e.g. human) TGFβR1, (1b) a transmembrane component comprising a transmembrane domain from (e.g. human) IL-2Rβ, and (1c) an intracellular component comprising a cytoplasmic/signaling domain from (e.g. human) IL-2Rβ; and (2) the second fusion protein comprises (2a) an extracellular component comprising an extracellular domain from (e.g. human) TGFβR2, (2b) a transmembrane component comprising a transmembrane domain from (e.g. human) IL-2Rγ, and (2c) an intracellular component comprising a cytoplasmic/signaling domain from (e.g. human) IL-2Rγ,
wherein, optionally, polynucleotide further encodes an antigen-binding protein such as a TCR or a CAR.

Embodiment 69a. The host cell of any one of Embodiments 65a-67a, wherein: (1) the first fusion protein comprises (1a) an extracellular component comprising an extracellular domain from (e.g. human) TGFβR2, (1b) a transmembrane component comprising a transmembrane domain from (e.g. human) TGFβR2, and (1c) an intracellular component comprising a cytoplasmic/signaling domain from (e.g. human) IL-2Rβ, and an optional TGFβR2 intracellular overhang or overhang sequence, e.g. as described for Table A and optionally comprising, consisting essentially or, or consisting of the amino acids R-V-N; and (2) the second fusion protein comprises (2a) an extracellular component comprising an extracellular domain from (e.g. human) TGFβR1, (2b) a transmembrane component comprising a transmembrane domain from (e.g. human) TGFβR1, and (2c) an intracellular component comprising a cytoplasmic/signaling domain from (e.g. human) IL-2R7, optionally wherein (i) the intracellular component further comprises a TGFβRI intracellular overhang or overhang sequence, e.g. as described for Table A and optionally comprising, consisting essentially or, or consisting of the amino acids C-H-N, disposed N-terminal to the IL-2Rγ cytoplasmic/signaling domain and/or (ii) the polynucleotide further encodes an antigen-binding protein such as a TCR or a CAR.

Embodiment 70a. The host cell of any one of Embodiments 65a-69a, wherein:
(i) the first fusion protein comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.:5, 10, 13, and 18; and
(ii) the second fusion protein comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 6, 9, 14, and 17.

Embodiment 71a. The host cell of Embodiment 70a, wherein the first fusion protein and the second fusion protein comprise an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NOs: (i) 5 and 6, respectively; (ii) 5 and 9, respectively; (iii) 5 and 14, respectively; (iv) 5 and 17, respectively; (v) 10 and 6, respectively; (vi) 10 and 9, respectively; (vii) 10 and 14, respectively; (viii) 10 and 17, respectively; (ix) 13 and 6, respectively; (x) 13 and 9, respectively; (xi) 13 and 14, respectively; (xii) 13 and 17, respectively; (xiii) 18 and 6, respectively; (xiv) 18 and 9, respectively; (xv) 18 and 14, respectively; or (xvi) 18 and 17, respectively.

Embodiment 72a. The host cell of any one of Embodiments 66a-71a, wherein the host cell encodes an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.:7, 11, 15, and 19.

Embodiment 73a. The host cell of any one of Embodiments 66a-72a, wherein the host cell expresses at its cell surface a protein complex comprising the first fusion protein and the second fusion protein, wherein the protein complex is capable of binding to a TGFβ polypeptide, which is optionally comprised in a TGFβ dimer.

Embodiment 74a. The host cell of any one of Embodiments 62a-73a, wherein the host cell comprises a hematopoietic progenitor cell or a human immune system cell.

Embodiment 75a. The host cell of any one of Embodiments 62a-74a, wherein the host cell comprises a T cell, such as a CD4+ T cell, a CD8+ T cell, a CD4− CD8− double negative T cell, or a γδ T cell, a natural killer cell, a natural killer T cell, a dendritic cell, or any combination thereof.

Embodiment 76a. The host cell of Embodiment any one of Embodiments 62a-75a, wherein the host cell comprises a T cell, wherein, optionally, the T cell comprises a naïve T cell, a central memory T cell, a stem cell memory T cell, an effector memory T cell, or any combination thereof.

Embodiment 77a. The host cell of any one of Embodiments 62a-76a, comprising a chromosomal gene knockout or a mutation of: a TGFβRI gene locus, a TGFβR2 gene locus, a PD-1 gene locus, a CTLA4 gene locus, a LAT gene locus, a TIM-3 gene locus, a PD-Li gene locus, a TIGIT gene locus, an A2AR gene locus, a Fas locus, a FasL gene locus, a B7-H3 gene locus, a B7-H4 gene locus, an IDO gene locus, a VISTA gene locus, a SIGLEC7 gene locus, a SIGLEC9 gene locus, a TRAC gene locus, a TRBC gene locus, a T cell receptor gene locus, a MHC (e.g. HLA) gene locus, a CBLB gene locus, a RASA2 gene locus, a UBASH3A gene locus, a CISH gene locus, or any combination thereof.

Embodiment 78a. The host cell of any one of Embodiments 62a-77a, wherein the host cell is modified (e.g., having a chromosomal knockout mutation and/or a chromosomal missense mutation and/or a chromosomal splice junction mutation; encoding an inhibitory nucleic acid such as an siRNA or an antisense oligonucleotide) to have reduced protein expression (including null expression), of an endogenous TGFβR1, an endogenous TGFβR2, or both, as compared to the unmodified host cell.

Embodiment 79a. The host cell of any one of Embodiments 62a-78a, comprising a polynucleotide encoding an antigen-binding protein.

Embodiment 80a. The host cell of any one of Embodiments 62a-79a, which expresses a/the antigen-binding protein.

Embodiment 81a. The host cell of Embodiment 79a or 80a, wherein the antigen-binding protein comprises a TCR (optionally wherein the TCR is endogenous to the host cell), an antigen-binding fragment of a TCR (e.g. a scTv), a scTCR, a CAR, or any combination thereof.

Embodiment 82a. The host cell of any one of Embodiments 79a-81a, wherein the antigen-binding protein binds to: a cancer antigen; an autoimmune antigen; a viral antigen; a bacterial antigen; a fungal antigen; a parasitic antigen; or any combination thereof.

Embodiment 83a. The host cell of Embodiment 82a, wherein the cancer comprises a solid tumor, a hematological malignancy, or both.

Embodiment 84a. The host cell of any one of Embodiments 82a-83a, wherein the antigen is selected from a WT1, mesothelin, KRAS, ROR1, EGFR, EGFRvIII, EGP-2, EGP-40, GD2, GD3, HPV E6, HPV E7, Her2, Li-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA 125, c-MET, FcRH5, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, NY-ESO, 5T4, BCMA, FAP, Carbonic anhydrase 9, BRAF, α-fetoprotein, MAGE-A3, MAGE-A4, SSX-2, PRAME, HA-1, p2M, ETA, tyrosinase, NRAS, or CEA antigen, optionally in complex with a MHC (e.g. HLA) molecule.

Embodiment 85a. The fusion protein of any one of Embodiments 1a-34a, the polynucleotide of any one of Embodiments 35a-55a, the vector of any one of Embodiments 56a-61a, or the host cell of any one of Embodiments 62a-84a, wherein the TGFβ polypeptide comprises a TGFB1, a TGFB2, a TGFB3, or any combination thereof, optionally comprised in a TGFβ polypeptide dimer.

Embodiment 86a. The host cell of any one of Embodiments 62a-85a, wherein the host cell comprises an increased level of phosphorylated STAT5 when the host cell is in the presence of a TGFβ (e.g., a TGFβ polypeptide dimer), as compared to the level of phosphorylated STAT5 when the host cell is not in the presence of the TGFβ.

Embodiment 87a. The host cell of any one of Embodiments 62a-86a, wherein the host cell comprises an IL-2R signal when the host cell is in the presence of a TGFβ polypeptide (e.g., a TGFβ polypeptide dimer).

Embodiment 88a. The host cell of any one of Embodiments 62a-87a, wherein the host cell performs one or more of the following when in the presence of a TGFβ polypeptide (e.g., a TGFβ polypeptide dimer): proliferation; growth; expression of an effector molecule (e.g., a T cell receptor); glycolytic metabolism; expression of protein (e.g., MYC, SLC7A5, or both); and biosynthesis of cholesterol.

Embodiment 89a. The host cell of any one of Embodiments 62a-88a, wherein the host cell has a reduced level of phosphorylated SMAD2/SMAD3 when the host cell is in the presence of a TGFβ polypeptide (e.g., a TGFβ polypeptide dimer), as compared to a reference cell,
wherein the reference cell does not encode or express the fusion protein(s), and, optionally, is not modified to have reduced protein expression (including null expression), of an endogenous TGFβR1, an endogenous TGFβR2, or both, as compared to the reference cell without the modification.

Embodiment 90a. The host cell of any one of Embodiments 62a-89a, wherein the host cell comprises an increased level of phosphorylated STAT5 when the host cell is in the presence of a TGFβ (e.g., a TGFβ polypeptide dimer), as compared to the level of phosphorylated STAT5 comprised in a reference cell not encoding or expressing the fusion protein(s), when the reference cell is in the presence of the TGFβ.

Embodiment 91a. The host cell of any one of Embodiments 62a-90a, wherein the host cell comprises an increased level of phosphorylated STAT5 when the host cell is in the presence of an IL-2, as compared to the level of phosphorylated STAT5 comprised in a reference cell that does not encode or express the fusion protein(s), respectively, when the reference cell is in the presence of the IL-2.

Embodiment 92a. The host cell of any one of Embodiments 62a-91a, wherein the host cell, when in the presence of a TGFβ polypeptide (optionally comprised in a TGFβ polypeptide dimer) and optionally not in the presence of an IL-2 polypeptide, is capable of killing antigen-expressing cells at a level that is about the same as the level of killing by a reference cell when the reference cell is in the presence of an IL-2 polypeptide.

Embodiment 93a. The host cell of any one of Embodiments 62a-92a, which is capable of localizing to a tumor in a host subject comprising the tumor, and optionally has greater localization at/in the tumor as compared to a reference host cell not expressing the fusion protein(s).

Embodiment 94a. A composition comprising: (i) the fusion protein of any one of Embodiments 1a-34a and 85a; and/or (ii) the polynucleotide of any one of Embodiments 35a-55a and 85a; and/or (iii) the vector of any one of Embodiments 56a-61a and 85a; and/or (iv) the host cell of any one of Embodiments 62a-93a, and a pharmaceutically acceptable carrier, excipient, or diluent.

Embodiment 95a. The composition of Embodiment 94a, comprising (i) a composition comprising at least about 30% CD4+T host cells, combined with (ii) a composition comprising at least about 30% CD8+T host cells, in about a 1:1 ratio.

Embodiment 96a. A method of treating a disease or condition in a subject, the method comprising administering to the subject an effective amount of: (i) the fusion protein of any one of Embodiments 1a-34a and 85a; and/or (ii) the polynucleotide of any one of Embodiments 35a-55a and 85a; and/or (iii) the vector of any one of Embodiments 56a-61a and 85a; and/or (iv) the host cell of any one of Embodiments 62a-93a; and/or (v) the composition of Embodiment 94a or 95a.

Embodiment 97a. The fusion protein of any one of Embodiments 1a-34a and 85a, and/or the polynucleotide of any one of Embodiments 35a-55a and 85a, and/or the vector of any one of Embodiments 56a-61a and 85a, and/or the host cell of any one of Embodiments 62a-93a, and/or the composition of Embodiment 94a or 95a, for use in a method of treating a disease or condition in a subject.

Embodiment 98a. The fusion protein of any one of Embodiments 1a-34a and 85a, and/or the polynucleotide of any one of Embodiments 35a-55a and 85a, and/or the vector of any one of Embodiments 56a-61a and 85a, and/or the host cell of any one of Embodiments 62a-93a, and/or the composition of Embodiment 94a or 95a, for use in a the preparation of a medicament for treating a disease or condition in a subject.

Embodiment 99a. The method of Embodiment 96a or the fusion protein, polynucleotide, vector, host cell, or composition for use of Embodiment 97a or 98a, wherein the disease or condition is associated with or characterized by expression of an antigen, wherein the antigen is specifically bound by the antigen-binding protein.

Embodiment 100a. The method of Embodiment 96a or 99a, or the fusion protein, polynucleotide, vector, host cell, or composition for use of any one of Embodiments 97a-99a, wherein the disease or condition comprises or is a hyperproliferative disease, a proliferative disease, an autoimmune disease, a neurodegenerative disease, or an infection.

Embodiment 101a. The method of Embodiment 96a, 99a, or 100a or the fusion protein, polynucleotide, vector, host cell, or composition for use of any one of Embodiments 97a-100a, wherein the disease or condition is a cancer, such as a solid tumor or a hematological malignancy.

Embodiment 102a. The method of Embodiment 101a or the fusion protein, polynucleotide, vector, host cell, or composition for use of Embodiment 101a, wherein the cancer comprises a carcinoma, a sarcoma, a glioma, a lymphoma, a leukemia, a myeloma, or any combination thereof.

Embodiment 103a. The method of Embodiment 101a or 102a, or the fusion protein, polynucleotide, vector, host cell, or composition for use of Embodiment 10 1a or 102a, wherein the cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

Embodiment 104a. The method of any one of Embodiments 101a-103a, or the fusion protein, polynucleotide, vector, host cell, or composition for use of any one of Embodiments 101a-103a, wherein the cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenstrom's macroglobulinemia, CD37 dendritic cell lymphoma, lymphoplasmacytic lymphoma, splenic marginal zone lymphoma, extranodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

Embodiment 105a. The method of Embodiment 101a or the fusion protein, polynucleotide, vector, host cell, or composition for use of Embodiment 101a, wherein the cancer comprises a solid tumor.

Embodiment 106a. The method of Embodiment 105a or the fusion protein, polynucleotide, vector, host cell, or composition for use of Embodiment 105a, wherein the solid tumor is a sarcoma or a carcinoma.

Embodiment 107a. The method of Embodiment 106a or the fusion protein, polynucleotide, vector, host cell, or composition for use of Embodiment 106a, wherein the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

Embodiment 108a. The method of any one of Embodiments 105a-107a or the fusion protein, polynucleotide, vector, host cell, or composition for use of any one of Embodiments 105a-107a, wherein the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma).

Embodiment 109a. The method of any one of Embodiments 105a-108a, or the fusion protein, polynucleotide, vector, host cell, or composition for use of any one of Embodiments 105a-108a, wherein the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

Embodiment 110a. The method of any one of Embodiments 96a and 99a-108a, or the fusion protein, polynucleotide, vector, host cell, or composition for use of any one of Embodiments 97a-108a, wherein the host cell is an allogeneic cell, a syngeneic cell, or an autologous cell.

Embodiment 111a. The method of any one of Embodiments 96a and 99a-110a, or the fusion protein, polynucleotide, vector, host cell, or composition for use of any one of Embodiments 97a-110a, wherein the method comprises administering a plurality of doses of the fusion protein, polynucleotide, vector, host cell, or composition to the subject.

Embodiment 112a. The method of Embodiment 110a or the fusion protein, polynucleotide, vector, host cell, or composition for use of Embodiment 1 1a, wherein the plurality of doses are administered at intervals between administrations of about two, three, four, five, six, seven, eight, or more weeks.

Embodiment 113a. The method of Embodiment 111a or 112a, or the fusion protein, polynucleotide, vector, host cell, or composition for use of Embodiment 111a or 112a, wherein a dose of the host cell comprises about 10⁵ cells/m² to about 10¹¹ cells/m².

Embodiment 114a. The method of any one of Embodiments 96a and 99a-113a, or the fusion protein, polynucleotide, vector, host cell, or composition for use of any one of Embodiments 97a-113a, wherein the subject is receiving, has received, or will receive one or more of: chemotherapy; (ii) radiation therapy; (iii) an inhibitor of an immune suppression component; (iv) an agonist of a stimulatory immune checkpoint agent; (v) RNAi; (vi) a cytokine; (vii) a surgery; (viii) a monoclonal antibody and/or an antibody-drug conjugate; or (ix) any combination of (i)-(viii), in any order.

Embodiment 115a. The method of any one of Embodiments 96a and 99a-114a, or the fusion protein, polynucleotide, vector, host cell, or composition for use of any one of Embodiments 97a-114a, wherein the subject is receiving, has received, or will receive: (i) IL-2; (ii) TGFβ; or (iii) both (i) and (ii).

Embodiment 116a. A method comprising introducing the polynucleotide of any one of Embodiments 35a-55a or the vector of any one of Embodiments 56a-61a into a host cell, wherein, optionally, the host cell comprises a T cell.

Embodiment 117a. The method of Embodiment 116a, further comprising incubating the host cell with TGFβ and, optionally, IL-2.

TABLE of
Sequences (Sequence Listing)

| SEQ ID | | |
|---|---|---|
| NO. | Description | Sequence |
| 1 | Human TGF-beta | MEAAVAAPRP RLLLLVLAAA AAAAAALLPG ATALQCFCHL |
| | receptor type-1, | CTKDNFTCVT DGLCFVSVTE TTDKVIHNSM CIAEIDLIPR |
| | isoform 1 | DRPFVCAPSS KTGSVTTTYC CNQDHCNKIE LPTTVKSSPG |
| | (UniProt KB | LGPVELAAVI AGPVCFVCIS LMLMVYICHN RTVIHHRVPN |
| | P36897-1), with | EEDPSLDRPF ISEGTTLKDL IYDMTTSGSG SGLPLLVQRT |
| | signal peptide | IARTIVLQES IGKGRFGEVW RGKWRGEEVA VKIFSSREER |
| | | SWFREAEIYQ TVMLRHENIL GFIAADNKDN GTWTQLWLVS |
| | | DYHEHGSLFD YLNRYTVTVE GMIKLALSTA SGLAHLHMEI |
| | | VGTQGKPAIA HRDLKSKNIL VKKNGTCCIA DLGLAVRHDS |
| | | ATDTIDIAPN HRVGTKRYMA PEVLDDSINM KHFESFKRAD |
| | | IYAMGLVFWE IARRCSIGGI HEDYQLPYYD LVPSDPSVEE |
| | | MRKVVCEQKL RPNIPNRWQS CEALRVMAKI MRECWYANGA |
| | | ARLTALRIKK TLSQLSQQEG IKM |
| 2 | Human TGF-beta | MGRGLLRGLW PLHIVLWTRI ASTIPPHVQK SVNNDMIVTD |
| | receptor type-2, | NNGAVKFPQL CKFCDVRFST CDNQKSCMSN CSITSICEKP |
| | isoform 1 | QEVCVAVWRK NDENITLETV CHDPKLPYHD FILEDAASPK |
| | (UniProt KB | CIMKEKKKPG ETFFMCSCSS DECNDNIIFS EEYNTSNPDL |
| | P37173-1), with | LLVIFQVTGI SLIPPLGVAI SVIIIFYCYR VNRQQKLSST |
| | signal peptide | WETGKTRKLM EFSEHCAIIL EDDRSDISST CANNINHNTE |
| | | LLPIELDTLV GKGRFAEVYK AKLKQNTSEQ FETVAVKIFP |
| | | YEEYASWKTE KDIFSDINLK HENILQFLTA EERKTELGKQ |
| | | YWLITAFHAK GNLQEYLTRH VISWEDLRKL GSSLARGIAH |
| | | LHSDHTPCGR PKMPIVHRDL KSSNILVKND LTCCLCDFGL |
| | | SLRLDPTLSV DDLANSGQVG TARYMAPEVL ESRMNLENVE |
| | | SFKQTDVYSM ALVLWEMTSR CNAVGEVKDY EPPFGSKVRE |
| | | HPCVESMKDN VLRDRGRPEI PSFWLNHQGI QMVCETLTEC |
| | | WDHDPEARLT AQCVÅERFSE LEHLDRLSGR SCSEEKIPED |
| | | GSLNTTK |
| 3 | Human | MAAPALSWRL PLLILLLPLA TSWASAAVNG TSQFTCFYNS |
| | Interleukin-2 | RANISCVWSQ DGALQDTSCQ VHAWPDRRRW NQTCELLPVS |
| | receptor subunit | QASWACNLIL GAPDSQKLTT VDIVTLRVLC REGVRWRVMA |
| | beta (UniProt KB | IQDFKPFENL RLMAPISLQV VHVETHRQNI SWEISQASHY |
| | P14784), with | FERHLEFEAR TLSPGHTWEE APLLTLKQKQ EWICLETLTP |
| | signal peptide | DTQYEFQVRV KPLQGEFTTW SPWSQPLAFR TKPAALGKDT |
| | | IPWLGHLLVG LSGAFGFIIL VYLLINCRNT GPWLKKVLKC |
| | | NTPDPSKFFS QLSSEHGGDV QKWLSSPFPS SSFSPGGLAP |
| | | EISPLEVLER DKVTQLLLQQ DKVPEPASLS SNHSLTSCFT |
| | | NQGYFFFHLP DALEIEACQV YFTYDPYSEE DPDEGVAGAP |
| | | TGSSPQPLQP LSGEDDAYCT FPSRDDLLLF SPSLLGGPSP |
| | | PSTAPGGSGA GEERMPPSLQ ERVPRDWDPQ PLGPPTPGVP |
| | | DLVDFQPPPE LVLREAGEEV PDAGPREGVS FPWSRPPGQG |
| | | EFRALNARLP LNTDAYLSLQ ELQGQDPTHL V |
| 4 | Human | MLKPSLPFTS LLFLQLPLLG VGLNTTILTP NGNEDTTADF |
| | Interleukin-2 | FLTTMPTDSL SVSTLPLPEV QCFVFNVEYM NCTWNSSSEP |
| | receptor subunit | QPTNLTLHYW YKNSDNDKVQ KCSHYLFSEE ITSGCQLQKK |
| | gamma, isoform | EIHLYQTFVV QLQDPREPRR QATQMLKLQN LVIPWAPENL |
| | 1 (UniProt KB | TLHKLSESQL ELNWNNRFLN HCLEHLVQYR TDWDHSWTEQ |
| | P31785-1), with | SVDYRHKFSL PSVDGQKRYT FRVRSRFNPL CGSAQHWSEW |
| | signal peptide | SHPIHWGSNT SKENPFLFAL EAVVISVGSM GLIISLLCVY |
| | | FWLERTMPRI PTLKNLEDLV TEYHGNFSAW SGVSKGLAES |
| | | LQPDYSERLC LVSEIPPKGG ALGEGPGASP CNQHSPYWAP |
| | | PCYTLKPET |
| 5 | TGFβR2_IL-2Rβ | TIPPHVQKSVNNDMIVTDNNGAVKFPQLCKFCDVRFSTCDNQKSC |
| | chimeric protein | MSNCSITSICEKPQEVCVAVWRKNDENITLETVCHDPKLPYHDFILED |
| | (with IL-2Rβ | AASPKCIMKEKKKPGETFFMCSCSSDECNDNIIFSEEYNTSNPDIPWL |
| | transmembrane | GHLLVGLSGAFGFIILVYLLINCRNTGPWLKKVLKCNTPDPSKFFSQLS |
| | domain) | SEHGGDVQKWLSSPFPSSSFSPGGLAPEISPLEVLERDKVTQLLLQQ |
| | | DKVPEPASLSSNHSLTSCFTNQGYFFFHLPDALEIEACQVYFTYDPYS |
| | | EEDPDEGVAGAPTGSSPQPLQPLSGEDDAYCTFPSRDDLLLFSPSLL |
| | | GGPSPPSTAPGGSGAGEERMPPSLQERVPRDWDPQPLGPPTPGVP |
| | | DLVDFQPPPELVLREAGEEVPDAGPREGVSFPWSRPPGQGEFRALN |
| | | ARLPLNTDAYLSLQELQGQDPTHLV |
| 6 | TGFBR1_IL-2Rγ | LQCFCHLCTKDNFTCVTDGLCFVSVTETTDKVIHNSMCIAEIDLIPRD |
| | chimeric protein | RPFVCAPSSKTGSVTTTYCCNQDHCNKIELPTTVKSSPGLGPVEPFLF |
| | (with IL-2Rγ | ALEAVVISVGSMGLIISLLCVYFWLERTMPRIPTLKNLEDLVTEYHGN |
| | transmembrane | FSAWSGVSKGLAESLQPDYSERLCLVSEIPPKGGALGEGPGASPCNQ |
| | domain) | HSPYWAPPCYTLKPET |
| 7 | TGFβR2_IL-2Rβ | MGRGLLRGLWPLHIVLWTRIASTIPPHVQKSVNNDMIVTDNNGAV |
| | linked to | KFPQLCKFCDVRFSTCDNQKSCMSNCSITSICEKPQEVCVAVWRKN |
| | TGFβR1_IL-2Rγ | DENITLETVCHDPKLPYHDFILEDAASPKCIMKEKKKPGETFFMCSCS |
| | by furin-linker- | SDECNDNIIFSEEYNTSNPDIPWLGHLLVGLSGAFGFIILVYLLINCRNT |
| | P2A cleavage | GPWLKKVLKCNTPDPSKFFSQLSSEHGGDVQKWLSSPFPSSSFSPG |
| | sequence, with | GLAPEISPLEVLERDKVTQLLLQQDKVPEPASLSSNHSLTSCFTNQGY |
| | signal peptides | FFFHLPDALEIEACQVYFTYDPYSEEDPDEGVAGAPTGSSPQPLQPLS |
| | | GEDDAYCTFPSRDDLLLFSPSLLGGPSPPSTAPGGSGAGEERMPPSL |
| | | QERVPRDWDPQPLGPPTPGVPDLVDFQPPPELVLREAGEEVPDAG |
| | | PREGVSFPWSRPPGQGEFRALNARLPLNTDAYLSLQELQGQDPTHL |
| | | VRAKRSGSGATNFSLLKQAGDVEENPGPMEAAVAAPRPRLLLLVLA |
| | | AAAAAAAALLPGATALQCFCHLCTKDNFTCVTDGLCFVSVTETTDKV |
| | | IHNSMCIAEIDLIPRDRPFVCAPSSKTGSVTTTYCCNQDHCNKIELPTT |
| | | VKSSPGLGPVEPFLFALEAVVISVGSMGLIISLLCVYFWLERTMPRIPT |
| | | LKNLEDLVTEYHGNFSAWSGVSKGLAESLQPDYSERLCLVSEIPPKG |
| | | GALGEGPGASPCNQHSPYWAPPCYTLKPET |
| 8 | Polynucleotide | ATGGGTCGGGGGCTGCTCAGGGGCCTGTGGCCGCTGCACATCGT |
| | encoding | CCTGTGGACGCGTATCGCCAGCACGATCCCACCGCACGTTCAGA |
| | [TGFβR2_IL-2Rβ | AGTCGGTTAATAACGACATGATAGTCACTGACAACAACGGTGCA |
| | linked to | GTCAAGTTTCCACAACTGTGTAAATTTTGTGÅTGTGAGATTTTCCA |
| | TGFβR1_IL-2Rγ | CCTGTGACAACCAGAAATCCTGCATGAGCAACTGCAGCATCACCT |
| | by furin-linker- | CCATCTGTGAGAAGCCACAGGAAGTCTGTGTGGCTGTATGGAGA |
| | P2A cleavage | AAGAATGACGAGAACATAACACTAGAGACAGTTTGCCATGACCC |
| | sequence, with | CAAGCTCCCCTACCATGACTTTATTCTGGAAGATGCTGCTTCTCCA |
| | signal peptides] | AAGTGCATTATGAAGGAGAAGAAAAAGCCTGGTGAGACTTTCTT |
| | | CATGTGTTCCTGTAGCTCTGATGAGTGCAATGACAACATCATCTT |
| | | CTCAGAAGAATATAACACCAGCAATCCTGACATTCCGTGGCTCGG |
| | | CCACCTCCTCGTGGGCCTCAGCGGGGCTTTTGGCTTCATCATCTTA |
| | | GTGTACTTGCTGATCAACTGCAGGAACACCGGGCCATGGCTGAA |
| | | GAAGGTCCTGAAGTGTAACACCCCAGACCCCTCGAAGTTCTTTTC |
| | | CCAGCTGAGCTCAGAGCATGGAGGAGACGTCCAGAAGTGGCTCT |
| | | CTTCGCCCTTCCCCTCATCGTCCTTCAGCCCTGGCGGCCTGGCACC |
| | | TGAGATCTCGCCACTAGAAGTGCTGGAGAGGGACAAGGTGACG |
| | | CAGCTGCTCCTGCAGCAGGACAAGGTGCCTGAGCCCGCATCCTT |
| | | AAGCAGCAACCACTCGCTGACCAGCTGCTTCACCAACCAGGGTTA |
| | | CTTCTTCTTCCACCTCCCGGATGCCTTGGAGATAGAGGCCTGCCA |
| | | GGTGTACTTTACTTACGACCCCTACTCAGAGGAAGACCCTGATGA |
| | | GGGTGTGGCCGGGGCACCCACAGGGTCTTCCCCCCAACCCCTGC |
| | | AGCCTCTGTCAGGGGAGGACGACGCCTACTGCACCTTCCCCTCCA |
| | | GGGATGACCTGCTGCTCTTCTCCCCCAGTCTCCTCGGTGGCCCCA |
| | | GCCCCCCAAGCACTGCCCCTGGGGGCAGTGGGGCCGGTGAAGA |
| | | GAGGATGCCCCCTTCTTTGCAAGAAAGAGTCCCCAGAGACTGGG |
| | | ACCCCCAGCCCCTGGGGCCTCCCACCCCAGGAGTCCCAGACCTG |
| | | GTGGATTTTCAGCCACCCCCTGAGCTGGTGCTGCGAGAGGCTGG |
| | | GGAGGAGGTCCCTGACGCTGGCCCCAGGGAGGGAGTCAGTTTC |
| | | CCCTGGTCCAGGCCTCCTGGGCAGGGGGAGTTCAGGGCCCTTAA |
| | | TGCTCGCCTGCCCCTGAACACTGATGCCTACTTGTCCCTCCAAGA |
| | | ACTCCAGGGTCAGGACCCAACTCACTTGGTGGGGGCCAAGCGGT |
| | | CCGGATCCGGAGCCACGAACTTCTCTCTGTTAAAGCAAGCAGGA |
| | | GACGTGGAAGAAAACCCCGGTCCCATGGAGGCGGCGGTCGCTG |
| | | CTCCGCGTCCCCGGCTGCTCCTCCTTGTGCTTGCTGCAGCGGCAG |
| | | CAGCTGCAGCGGCACTTCTCCCGGGGGCGACGGCGTTACAGTGT |
| | | TTCTGCCACCTCTGTACAAAAGACAATTTTACTTGTGTGACAGAT |
| | | GGGCTCTGCTTTGTCTCTGTCACAGAGACCACCGACAAAGTGATC |
| | | CACAACAGCATGTGCATCGCGGAGATCGACCTGATTCCTCGGGA |
| | | CCGCCCCTTCGTGTGTGCACCCTCTTCAAAAACTGGGTCTGTGAC |
| | | TACAACATATTGCTGCAATCAGGACCATTGCAATAAAATAGAACT |
| | | TCCAACTACTGTAAAGTCATCACCTGGCCTTGGTCCTGTGGAACC |
| | | TTTCCTGTTTGCATTGGAAGCCGTGGTTATCTCTGTTGGCTCCATG |
| | | GGATTGATTATCAGCCTTCTCTGTGTGTATTTCTGGCTGGAACGG |
| | | ACGATGCCCCGAATTCCCACCCTGAAGAACCTAGAGGATCTTGTT |
| | | ACTGAATACCACGGGAACTTTTCGGCCTGGAGTGGTGTGTCTAA |
| | | GGGACTGGCTGAGAGTCTGCAGCCAGACTACAGTGAACGACTCT |
| | | GCCTCGTCAGTGAGATTCCCCCAAAAGGAGGGGCCCTTGGGGAG |
| | | GGGCCTGGGGCCTCCCCATGCAACCAGCATAGCCCCTACTGGGC |
| | | CCCCCCATGTTACACCCTAAAGCCTGAAACC |
| 9 | TGFβR1_IL-2Rβ | LQCFCHLCTKDNFTCVTDGLCFVSVTETTDKVIHNSMCIAEIDLIPRD |
| | chimeric protein | RPFVCAPSSKTGSVTTTYCCNQDHCNKIELPTTVKSSPGLGPVEIPWL |
| | (with IL-2Rβ | GHLLVGLSGAFGFIILVYLLINCRNTGPWLKKVLKCNTPDPSKFFSQLS |
| | transmembrane | SEHGGDVQKWLSSPFPSSSFSPGGLAPEISPLEVLERDKVTQLLLQQ |
| | domain) | DKVPEPASLSSNHSLTSCFTNQGYFFFHLPDALEIEACQVYFTYDPYS |
| | | EEDPDEGVAGAPTGSSPQPLQPLSGEDDAYCTFPSRDDLLLFSPSLL |
| | | GGPSPPSTAPGGSGAGEERMPPSLQERVPRDWDPQPLGPPTPGVP |
| | | DLVDFQPPPELVLREAGEEVPDAGPREGVSFPWSRPPGQGEFRALN |
| | | ARLPLNTDAYLSLQELQGQDPTHLV |
| 10 | TGFBR2_IL-2Rγ | TIPPHVQKSVNNDMIVTDNNGAVKFPQLCKFCDVRFSTCDNQKSC |
| | chimeric protein | MSNCSITSICEKPQEVCVAVWRKNDENITLETVCHDPKLPYHDFILED |
| | (with IL-2Rγ | AASPKCIMKEKKKPGETFFMCSCSSDECNDNIIFSEEYNTSNPDPFLF |
| | transmembrane | ALEAVVISVGSMGLIISLLCVYFWLERTMPRIPTLKNLEDLVTEYHGN |
| | domain) | FSAWSGVSKGLAESLQPDYSERLCLVSEIPPKGGALGEGPGASPCNQ |
| | | HSPYWAPPCYTLKPET |
| 11 | TGFβR1_IL-2Rβ | MEAAVAAPRPRLLLLVLAAAAAAAAALLPGATALQCFCHLCTKDNF |
| | linked to | TCVTDGLCFVSVTETTDKVIHNSMCIAEIDLIPRDRPFVCAPSSKTGSV |
| | TGFβR2_IL-2Rγ | TTTYCCNQDHCNKIELPTTVKSSPGLGPVEIPWLGHLLVGLSGAFGFI |
| | by furin-linker- | ILVYLLINCRNTGPWLKKVLKCNTPDPSKFFSQLSSEHGGDVQKWLS |
| | P2A cleavage | SPFPSSSFSPGGLAPEISPLEVLERDKVTQLLLQQDKVPEPASLSSNHS |
| | sequence, with | LTSCFTNQGYFFFHLPDALEIEACQVYFTYDPYSEEDPDEGVAGAPT |
| | signal peptides | GSSPQPLQPLSGEDDAYCTFPSRDDLLLFSPSLLGGPSPPSTAPGGSG |
| | | AGEERMPPSLQERVPRDWDPQPLGPPTPGVPDLVDFQPPPELVLR |
| | | EAGEEVPDAGPREGVSFPWSRPPGQGEFRALNARLPLNTDAYLSLQ |
| | | ELQGQDPTHLVRAKRSGSGATNFSLLKQAGDVEENPGPMGRGLLR |
| | | GLWPLHIVLWTRIASTIPPHVQKSVNNDMIVTDNNGAVKFPQLCKF |
| | | CDVRFSTCDNQKSCMSNCSITSICEKPQEVCVAVWRKNDENITLETV |
| | | CHDPKLPYHDFILEDAASPKCIMKEKKKPGETFFMCSCSSDECNDNII |
| | | FSEEYNTSNPDPFLFALEAVVISVGSMGLIISLLCVYFWLERTMPRIPT |
| | | LKNLEDLVTEYHGNFSAWSGVSKGLAESLQPDYSERLCLVSEIPPKG |
| | | GALGEGPGASPCNQHSPYWAPPCYTLKPET |
| 12 | Polynucleotide | atggaggcggcggtcgctgctccgcgtccccggctgctcctccttgtgcttgctgcagc |
| | encoding | ggcagcagctgcagcggcacttctcccgggggcgacggcgttacagtgtttctgccac |
| | [TGFβR1_IL-2Rβ | ctctgtacaaaagacaattttacttgtgtgacagatgggctctgctttgtctctgtcaca |
| | linked to | gagaccaccgacaaagtgatccacaacagcatgtgcatcgcggagatcgacctgatt |
| | TGFβR1_IL-2Rγ | cctcgggaccgccccttcgtgtgtgcaccctcttcaaaaactgggtctgtgactacaac |
| | by furin-linker- | atattgctgcaatcaggaccattgcaataaaatagaacttccaactactgtaaagtca |
| | P2A cleavage | tcacctggccttggtcctgtggaaattccgtggctcggccacctcctcgtgggcctcag |
| | sequence, with | cggggcttttggcttcatcatcttagtgtacttgctgatcaactgcaggaacaccgggc |
| | signal peptides] | catggctgaagaaggtcctgaagtgtaacaccccagacccctcgaagttcttttccca |
| | | gctgagctcagagcatggaggagacgtccagaagtggctctcttcgcccttcccctcat |
| | | cgtccttcagccctggoggcctggcacctgagatctcgccactagaagtgctggagag |
| | | ggacaaggtgacgcagctgctcctgcagcaggacaaggtgcctgagcccgcatoctt |
| | | aagcagcaaccactcgctgaccagctgcttcaccaaccagggttacttcttcttccacc |
| | | tcccggatgccttggagatagaggcctgccaggtgtactttacttacgacccctactca |
| | | gaggaagaccctgatgagggtgtggccggggcacccacagggtcttccccccaaccc |
| | | ctgcagcctctgtcaggggaggacgacgcctactgcaccttcccctccagggatgacc |
| | | tgctgctcttctcccccagtctcctcggtggccccagccccccaagcactgcccctggg |
| | | ggcagtggggccggtgaagagaggatgcccccttctttgcaagaaagagtccccaga |
| | | gactgggacccccagcccctggggoctcccaccccaggagtoccagacctggtggatt |
| | | ttcagccaccccctgagctggtgctgcgagaggctggggaggaggtccctgacgctgg |
| | | ccccagggagggagtcagtttcccctggtccaggcctcctgggcagggggagttcagg |
| | | gcccttaatgctcgcctgcccctgaacactgatgcctacttgtccctocaagaactcca |
| | | gggtcaggacccaactcacttggtgcgggccaagcggtcoggatccggagccacgaa |
| | | cttctctctgttaaagcaagcaggagacgtggaagaaaaccccggtcccatgggtog |
| | | ggggctgctcaggggcctgtggccgctgcacatcgtcctgtggacgcgtatcgccagc |
| | | acgatcccaccgcacgttcagaagtcggttaataacgacatgatagtcactgacaac |
| | | aacggtgcagtcaagtttccacaactgtgtaaattttgtgatgtgagattttccacctgt |
| | | gacaaccagaaatcctgcatgagcaactgcagcatcacctocatctgtgagaagcca |
| | | caggaagtctgtgtggctgtatggagaaagaatgacgagaacataacactagagac |
| | | agtttgccatgaccccaagctcccctaccatgactttattctggaagatgctgcttctcc |
| | | aaagtgcattatgaaggagaagaaaaagcctggtgagactttcttcatgtgttoctgt |
| | | agctctgatgagtgcaatgacaacatcatcttctcagaagaatataacaccagcaatcc |
| | | tgaccctttcctgtttgcattggaagccgtggttatctctgttggctccatgggattgat |
| | | tatcagccttctctgtgtgtatttctggctggaacggacgatgccccgaattcccaccct |
| | | gaagaacctagaggatcttgttactgaataccacgggaacttttcggcctggagtggt |
| | | gtgtctaagggactggctgagagtctgcagccagactacagtgaacgactctgcctcg |
| | | tcagtgagattcccccaaaaggaggggcccttggggaggggcctggggcctccccat |
| | | gcaaccagcatagcccctactgggcccccccatgttacaccctaaagcctgaaacc |
| 13 | TGFβR2-TM_IL- | TIPPHVQKSVNNDMIVTDNNGAVKFPQLCKFCDVRFSTCDNQKSC |
| | 2Rβ chimeric | MSNCSITSICEKPQEVCVAVWRKNDENITLETVCHDPKLPYHDFILED |
| | protein | AASPKCIMKEKKKPGETFFMCSCSSDECNDNIIFSEEYNTSNPDLLLVI |
| | | FQVTGISLLPPLGVAISVIIIFYCYNCRNTGPWLKKVLKCNTPDPSKFF |
| | | SQLSSEHGGDVQKWLSSPFPSSSFSPGGLAPEISPLEVLERDKVTQLL |
| | | LQQDKVPEPASLSSNHSLTSCFTNQGYFFFHLPDALEIEACQVYFTYD |
| | | PYSEEDPDEGVAGAPTGSSPQPLQPLSGEDDAYCTFPSRDDLLLFSP |
| | | SLLGGPSPPSTAPGGSGAGEERMPPSLQERVPRDWDPQPLGPPTP |
| | | GVPDLVDFQPPPELVLREAGEEVPDAGPREGVSFPWSRPPGQGEFR |
| | | ALNARLPLNTDAYLSLQELQGQDPTHLV |
| 14 | TGFβR1-TM-with | LQCFCHLCTKDNFTCVTDGLCFVSVTETTDKVIHNSMCIAEIDLIPRD |
| | intracellular | RPFVCAPSSKTGSVTTTYCCNQDHCNKIELPTTVKSSPGLGPVELAA |
| | overhang_IL-2Rγ | VIAGPVCFVCISLMLMVYICHNERTMPRIPTLKNLEDLVTEYHGNFS |
| | chimeric protein | AWSGVSKGLAESLQPDYSERLCLVSEIPPKGGALGEGPGASPCNQH |
| | | SPYWAPPCYTLKPET |
| 15 | TGFβR2-TM_IL- | MGRGLLRGLWPLHIVLWTRIASTIPPHVQKSVNNDMIVTDNNGAV |
| | 2Rβ linked to | KFPQLCKFCDVRFSTCDNQKSCMSNCSITSICEKPQEVCVAVWRKN |
| | TGFβR1-TM-with | DENITLETVCHDPKLPYHDFILEDAASPKCIMKEKKKPGETFFMCSCS |
| | intracellular | SDECNDNIIFSEEYNTSNPDLLLVIFQVTGISLLPPLGVAISVIIIFYCYN |
| | overhang_IL-2Rγ | CRNTGPWLKKVLKCNTPDPSKFFSQLSSEHGGDVQKWLSSPFPSSS |
| | by furin-linker- | FSPGGLAPEISPLEVLERDKVTQLLLQQDKVPEPÅSLSSNHSLTSCFTN |
| | P2A cleavage | QGYFFFHLPDALEIEACQVYFTYDPYSEEDPDEGVAGAPTGSSPQPL |
| | sequence, with | QPLSGEDDAYCTFPSRDDLLLFSPSLLGGPSPPSTAPGGSGAGEERM |
| | signal peptides | PPSLQERVPRDWDPQPLGPPTPGVPDLVDFQPPPELVLREAGEEVP |
| | | DAGPREGVSFPWSRPPGQGEFRALNARLPLNTDAYLSLQELQGQD |
| | | PTHLVRAKRSGSGATNFSLLKQAGDVEENPGPMEAAVAAPRPRLLL |
| | | LVLAAAAAAAAALLPGATALQCFCHLCTKDNFTCVTDGLCFVSVTET |
| | | TDKVIHNSMCIAEIDLIPRDRPFVCAPSSKTGSVTTTYCCNQDHCNKI |
| | | ELPTTVKSSPGLGPVELAAVIAGPVCFVCISLMLMVYICHNERTMPR |
| | | IPTLKNLEDLVTEYHGNFSAWSGVSKGLAESLQPDYSERLCLVSEIPP |
| | | KGGALGEGPGASPCNQHSPYWAPPCYTLKPET |
| 16 | Polynucleotide | ATGGGTCGGGGGCTGCTCAGGGGCCTGTGGCCGCTGCACATCGT |
| | encoding | CCTGTGGACGCGTATCGCCAGCACGATCCCACCGCACGTTCAGA |
| | [TGFβR2-TM_IL- | AGTCGGTTAATAACGACATGATAGTCACTGACAACAACGGTGCA |
| | 2Rβ linked to | GTCAAGTTTCCACAACTGTGTAAATTTTGTGATGTGAGATTTTCCA |
| | TGFβR1-TM-with | CCTGTGACAACCAGAAATCCTGCATGAGCAACTGCAGCATCACCT |
| | intracellular | CCATCTGTGAGAAGCCACAGGAÅGTCTGTGTGGCTGTATGGAGA |
| | overhang_IL-2Rγ | AAGAATGACGAGAACATAACACTAGAGACAGTTTGCCATGACCC |
| | by furin-linker- | CAAGCTCCCCTACCATGACTTTATTCTGGAAGATGCTGCTTCTCCA |
| | P2A cleavage | AAGTGCATTATGAAGGAGAAGAAAAAGCCTGGTGAGACTTTCTT |
| | sequence, with | CATGTGTTCCTGTAGCTCTGATGAGTGCAATGACAACATCATCTT |
| | signal peptides] | CTCAGAAGAATATAACACCAGCAATCCTGACTTGTTGCTAGTCAT |
| | | ATTTCAAGTGACAGGCATCAGCCTCCTGCCACCACTGGGAGTTGC |
| | | CATATCTGTCATCATCATCTTCTACTGCTACAACTGCAGGAACACC |
| | | GGGCCATGGCTGAAGAAGGTCCTGAAGTGTAACACCCCAGACCC |
| | | CTCGAAGTTCTTTTCCCAGCTGAGCTCAGAGCATGGAGGAGACG |
| | | TCCAGAAGTGGCTCTCTTCGCCCTTCCCCTCATCGTCCTTCAGCCC |
| | | TGGCGGCCTGGCACCTGAGATCTCGCCACTAGAAGTGCTGGAGA |
| | | GGGACAAGGTGACGCAGCTGCTCCTGCAGCAGGACAAGGTGCC |
| | | TGAGCCCGCATCCTTAAGCAGCAACCACTCGCTGACCAGCTGCTT |
| | | CACCAACCAGGGTTACTTCTTCTTCCACCTCCCGGATGCCTTGGA |
| | | GATAGAGGCCTGCCAGGTGTACTTTACTTACGACCCCTACTCAGA |
| | | GGAAGACCCTGATGAGGGTGTGGCCGGGGCACCCACAGGGTCT |
| | | TCCCCCCAACCCCTGCAGCCTCTGTCAGGGGAGGACGACGCCTAC |
| | | TGCACCTTCCCCTCCAGGGATGACCTGCTGCTCTTCTCCCCCAGTC |
| | | TCCTCGGTGGCCCCAGCCCCCCAAGCACTGCCCCTGGGGGCAGT |
| | | GGGGCCGGTGAAGAGAGGATGCCCCCTTCTTTGCAAGAAAGAGT |
| | | CCCCAGAGACTGGGACCCCCAGCCCCTGGGGCCTQCCACCCCAG |
| | | GAGTCCCAGACCTGGTGGATTTTCAGCCACCCCCTGAGCTGGTGC |
| | | TGCGAGAGGCTGGGGAGGAGGTCCCTGACGCTGGCCCCAGGGA |
| | | GGGAGTCAGTTTCCCCTGGTCCAGGCCTCCTGGGCAGGGGGAGT |
| | | TCAGGGCCCTTAATGCTCGCCTGCCCCTGAACACTGATGCCTACT |
| | | TGTCCCTCCAAGAACTCCAGGGTCAGGACCCAACTCACTTGGTGC |
| | | GGGCCAAGCGGTCCGGATCCGGAGCCACGAACTTCTCTCTGTTA |
| | | AAGCAAGCAGGAGACGTGGAAGAAAACCCCGGTCCCATGGAGG |
| | | CGGCGGTCGCTGCTCCGCGTCCCCGGCTGCTCCTCCTTGTGCTTG |
| | | CTGCAGCGGCAGCAGCTGCAGCGGCACTTCTCCCGGGGGCGACG |
| | | GCGTTACAGTGTTTCTGCCACCTCTGTACAAAAGACAATTTTACTT |
| | | GTGTGACAGATGGGCTCTGCTTTGTCTCTGTCACAGAGACCACCG |
| | | ACAAAGTGATCCACAACAGCATGTGCATCGCGGAGATCGACCTG |
| | | ATTCCTCGGGACCGCCCCTTCGTGTGTGCACCCTCTTCAAAAACT |
| | | GGGTCTGTGACTACAACATATTGCTGCAATCAGGACCATTGCAAT |
| | | AAAATAGAACTTCCAACTACTGTAAAGTCATCACCTGGCCTTGGT |
| | | CCTGTGGAACTGGCAGCTGTCATTGCTGGACCAGTGTGCTTCGTC |
| | | TGCATCTCACTCATGTTGATGGTCTATATCTGCCACAACGAACGG |
| | | ACGATGCCCCGAATTCCCACCCTGAAGAACCTAGAGGATCTTGTT |
| | | ACTGAATACCACGGGÅACTTTTCGGCCTGGAGTGGTGTGTCTAA |
| | | GGGACTGGCTGAGAGTCTGCAGCCAGACTACAGTGAACGACTCT |
| | | GCCTCGTCAGTGAGATTCCCCCAAAAGGAGGGGCCCTTGGGGAG |
| | | GGGCCTGGGGCCTCCCCATGCAACCAGCATAGCCCCTACTGGGC |
| | | CCCCCCATGTTACACCCTAAAGCCTGAAACC |
| 17 | TGFβR1-TM+3aa_ | LQCFCHLCTKDNFTCVTDGLCFVSVTETTDKVIHNSMCIAEIDLIPRD |
| | IL2Rβ | RPFVCAPSSKTGSVTTTYCCNQDHCNKIELPTTVKSSPGLGPVELAA |
| | chimeric protein | VIAGPVCFVCISLMLMVYICHNNCRNTGPWLKKVLKCNTPDPSKFF |
| | | SQLSSEHGGDVQKWLSSPFPSSSFSPGGLAPEISPLEVLERDKVTQLL |
| | | LQQDKVPEPASLSSNHSLTSCFTNQGYFFFHLPDALEIEACQVYFTYD |
| | | PYSEEDPDEGVAGAPTGSSPQPLQPLSGEDDAYCTFPSRDDLLLFSP |
| | | SLLGGPSPPSTAPGGSGAGEERMPPSLQERVPRDWDPQPLGPPTP |
| | | GVPDLVDFQPPPELVLREAGEEVPDAGPREGVSFPWSRPPGQGEFR |
| | | ALNARLPLNTDAYLSLQELQGQDPTHLV |
| 18 | TGFβR2-TM- | TIPPHVQKSVNNDMIVTDNNGAVKFPQLCKFCDVRFSTCDNQKSC |
| | without | MSNCSITSICEKPQEVCVAVWRKNDENITLETVCHDPKLPYHDFILED |
| | intracellular | AASPKCIMKEKKKPGETFFMCSCSSDECNDNIIFSEEYNTSNPDLLLVI |
| | overhang_IL2Rγ | FQVTGISLIPPLGVAISVIIIFYCYERTMPRIPTLKNLEDLVTEYHGNFS |
| | chimeric protein | AWSGVSKGLAESLQPDYSERLCLVSEIPPKGGALGEGPGASPCNQH |
| | | SPYWAPPCYTLKPET |
| 19 | TGFβR1-TM-with | MEAAVAAPRPRLLLLVLAAAAAAAAALLPGATALQCFCHLCTKDNF |
| | intracellular | TCVTDGLCFVSVTETTDKVIHNSMCIAEIDLIPRDRPFVCAPSSKTGSV |
| | overhang_IL-2Rβ | TTTYCCNQDHCNKIELPTTVKSSPGLGPVELAAVIAGPVCFVCISLML |
| | linked to | MVYICHNNCRNTGPWLKKVLKCNTPDPSKFFSQLSSEHGGDVQK |
| | TGFBR2-TM_IL- | WLSSPFPSSSFSPGGLAPEISPLEVLERDKVTQLLLQQDKVPEPASLSS |
| | 2Rγ by furin- | NHSLTSCFTNQGYFFFHLPDALEIEACQVYFTYDPYSEEDPDEGVAG |
| | linker-P2A | APTGSSPQPLQPLSGEDDAYCTFPSRDDLLLFSPSLLGGPSPPSTAPG |
| | cleavage | GSGAGEERMPPSLQERVPRDWDPQPLGPPTPGVPDLVDFQPPPEL |
| | sequence, with | VLREAGEEVPDAGPREGVSFPWSRPPGQGEFRALNARLPLNTDAYL |
| | signal peptides | SLQELQGQDPTHLVRAKRSGSGATNFSLLKQAGDVEENPGPMGRG |
| | | LLRGLWPLHIVLWTRIASTIPPHVQKSVNNDMIVTDNNGAVKFPQL |
| | | CKFCDVRFSTCDNQKSCMSNCSITSICEKPQEVCVAVWRKNDENITL |
| | | ETVCHDPKLPYHDFILEDAASPKCIMKEKKKPGETFFMCSCSSDECN |
| | | DNIIFSEEYNTSNPDLLLVIFQVTGISLLPPLGVAISVIIIFYCYERTMPR |
| | | IPTLKNLEDLVTEYHGNFSAWSGVSKGLAESLQPDYSERLCLVSEIPP |
| | | KGGALGEGPGASPCNQHSPYWAPPCYTLKPET |
| 20 | Polynucleotide | ATGGAGGCGGCGGTCGCTGCTCCGCGTCCCCGGCTGCTCCTCCTT |
| | encoding | GTGCTTGCTGCAGCGGCAGCAGCTGCAGCGGCACTTCTCCCGGG |
| | [TGFβR1-TM- | GGCGACGGCGTTACAGTGTTTCTGCCACCTCTGTACAAAAGACAA |
| | with intracellular | TTTTACTTGTGTGACAGATGGGCTCTGCTTTGTCTCTGTCACAGAG |
| | overhang_IL-2Rβ | ACCACCGACAAAGTGATCCACAACAGCATGTGCATCGCGGAGAT |
| | linked to | CGACCTGATTCCTCGGGACCGCCCCTTCGTGTGTGCACCCTCTTCA |
| | TGFBR2-TM_IL- | AAAACTGGGTCTGTGACTACAACATATTGCTGCAATCAGGACCAT |
| | 2Rγ by furin- | TGCAATAAAATAGAACTTCCAACTACTGTAAAGTCATCACCTGGC |
| | linker-P2A | CTTGGTCCTGTGGAACTGGCAGCTGTCATTGCTGGACCAGTGTGC |
| | cleavage | TTCGTCTGCATCTCACTCATGTTGATGGTCTATATCTGCCACAACA |
| | sequence, with | ACTGCAGGAACACCGGGCCATGGCTGAAGAAGGTCCTGAAGTGT |
| | signal peptides] | AACACCCCAGACCCCTCGAAGTTCTTTTCCCAGCTGAGCTCAGAG |
| | | CATGGAGGAGACGTCCAGAAGTGGCTCTCTTCGCCCTTCCCCTCA |
| | | TCGTCCTTCAGCCCTGGCGGCCTGGCACCTGAGATCTCGCCACTA |
| | | GAAGTGCTGGAGAGGGACAAGGTGACGCAGCTGCTCCTGCAGC |
| | | AGGACAAGGTGCCTGAGCCCGCATCCTTAAGCAGCAACCACTCG |
| | | CTGACCAGCTGCTTCACCAACCAGGGTTACTTCTTCTTCCACCTCC |
| | | CGGATGCCTTGGAGATAGAGGCCTGCCAGGTGTACTTTACTTAC |
| | | GACCCCTACTCAGAGGAAGACCCTGATGÅGGGTGTGGCCGGGG |
| | | CACCCACAGGGTCTTCCCCCCAACCCCTGCAGCCTCTGTCAGGGG |
| | | AGGACGACGCCTACTGCACCTTCCCCTCCAGGGATGACCTGCTGC |
| | | TCTTCTCCCCCAGTCTCCTCGGTGGCCCCAGCCCCCCAAGCACTGC |
| | | CCCTGGGGGCAGTGGGGCCGGTGAAGAGAGGATGCCCCCTTCTT |
| | | TGCAAGAAAGAGTCCCCAGAGACTGGGACCCCCAGCCCCTGGGG |
| | | CCTCCCACCCCAGGAGTCCCAGACCTGGTGGATTTTCAGCCACCC |
| | | CCTGAGCTGGTGCTGCGAGAGGCTGGGGAGGAGGTCCCTGACG |
| | | CTGGCCCCAGGGAGGGAGTCAGTTTCCCCTGGTCCAGGCCTCCT |
| | | GGGCAGGGGGAGTTCAGGGCCCTTAATGCTCGCCTGCCCCTGAA |
| | | CACTGATGCCTACTTGTCCCTCCAAGAACTCCAGGGTCAGGACCC |
| | | AACTCACTTGGTGCGGGCCAAGCGGTCCGGATCCGGAGCCACGA |
| | | ACTTCTCTCTGTTAAAGCAAGCAGGAGACGTGGAAGAAAACCCC |
| | | GGTCCCATGGGTCGGGGGCTGCTCAGGGGCCTGTGGCCGCTGCA |
| | | CATCGTCCTGTGGACGCGTATCGCCAGCACGATCCCACCGCACGT |
| | | TCAGAAGTCGGTTAATAACGACATGATAGTCACTGACAACAACG |
| | | GTGCAGTCAAGTTTCCACAACTGTGTAAATTTTGTGATGTGAGAT |
| | | TTTCCACCTGTGACAACCAGAAATCCTGCATGAGCAACTGCAGCA |
| | | TCACCTCCATCTGTGAGAAGCCACAGGAAGTCTGTGTGGCTGTAT |
| | | GGAGAAAGAATGACGAGAACATAACACTAGAGACAGTTTGCCAT |
| | | GACCCCAAGCTCCCCTACCATGACTTTATTCTGGAAGATGCTGCTT |
| | | CTCCAAAGTGCATTATGAAGGAGAAGAAAAAGCCTGGTGAGACT |
| | | TTCTTCATGTGTTCCTGTAGCTCTGATGAGTGCAATGACAACATCA |
| | | TCTTCTCAGAAGAATATAACACCAGCAATCCTGACTTGTTGCTAG |
| | | TCATATTTCAAGTGACAGGCATCAGCCTCCTGCCACCACTGGGAG |
| | | TTGCCATATCTGTCATCATCATCTTCTACTGCTACGAACGGACGAT |
| | | GCCCCGAATTCCCACCCTGAAGAACCTAGAGGATCTTGTTACTGA |
| | | ATACCACGGGAACTTTTCGGCCTGGAGTGGTGTGTCTAAGGGAC |
| | | TGGCTGAGAGTCTGCAGCCAGACTACAGTGAACGACTCTGCCTC |
| | | GTCAGTGAGATTCCCCCAAAAGGAGGGGCCCTTGGGGAGGGGC |
| | | CTGGGGCCTCCCCATGCAACCAGCATAGCCCCTACTGGGCCCCCC |
| | | CATGTTACACCCTAAAGCCTGAAACC |
| 21 | Signal peptide, | MEAAVAAPRPRLLLLVLAAAAAAAAALLPGATA |
| | TGFβR1 | |
| 22 | Signal peptide, | MGRGLLRGLWPLHIVLWTRIAS |
| | TGFβR2 | |
| 23 | Furin cleavage | R-X-K/R-R |
| | motif | |
| 24 | Porcine | GSGATNFSLLKQAGDVEENPGP |
| | teschovirus-1 2A | |
| | (P2A) peptide | |
| | with N-terminal | |
| | GSG linker | |
| 25 | Thoseaasigna | LEGGGEGRGSLLTCGDVEENPGPR |
| | virus 2A (T2A) | |
| | peptide | |
| 26 | Equine rhinitis A | QCTNYALLKLAGDVESNPGP |
| | virus (ERAV) 2A | |
| | (E2A) | |
| 27 | Foot-and-Mouth | GSGVKQTLNFDLLKLAGDVESNPGP |
| | disease virus 2A | |
| | (F2A) peptide | |
| | with N-terminal | |
| | GSG linker | |
| 28 | Furin cleavage | RAKRSGSGATNFSLLKQAGDVEENPGP |
| | motif-serine- | |
| | GSG-P2A | |
| 29 | Truncated NGFR | MGAGATGRAMDGPRLLLLLLLGVSLGGAKEACPTGLYTHSGECCKA |
| | (tNGFR), with | CNLGEGVAQPCGANQTVCEPCLDSVTFSDVVSATEPCKPCTECVGL |
| | signal peptide | QSMSAPCVEADDAVCRCAYGYYQDETTGRCEACRVCEAGSGLVFS |
| | | CQDKQNTVCEECPDGTYSDEANHVDPCLPCTVCEDTERQLRECTR |
| | | WADAECEEIPGRWITRSTPPEGSDSTAPSTQEPEAPPEQDLIASTVA |
| | | GVVTTVMGSSQPVVTRGTTDNLIPVYCSILAAVVVGLVAYIAFKRW |
| | | NS |
| 30 | Truncated EGFR | RKVCNGIGIGEFKDSLSINATNIKHFKNCTSISGDLHILPVAFRGDSFT |
| | (tEGFR) | HTPPLDPQELDILKTVKEITGFLLIQAWPENRTDLHAFENLEIIRGRTK |
| | | QHGQFSLAVVSLNITSLGLRSLKEISDGDVIISGNKNLCYANTINWKK |
| | | LFGTSGQKTKIISNRGENSCKATGQVCHALCSPEGCWGPEPRDCVS |
| | | CRNVSRGRECVDKCNLLEGEPREFVENSECIQCHPECLPQAMNITCT |
| | | GRGPDNCIQCAHYIDGPHCVKTCPAGVMGENNTLVWKYADAGHV |
| | | CHLCHPNCTYGCTGPGLEGCPTNGPKIPSIATGMVGALLLLLVVALGI |
| | | GLFM |
| 31 | Linker 218 amino | GSTSGSGKPGSGEGSTKG |
| | acid sequence | |
| 32 | linker amino acid | GSGKPGSGEG |
| | sequence | |
| 33 | linker amino acid | GKPGSGEG |
| | sequence | |
| 34 | linker amino acid | SGKPGSGE |
| | sequence | |
| 35 | linker amino acid | BPXXXZ, wherein each X is independently a glycine (G) or |
| | sequence | serine (S), B is a positively charged amino acid and Z is |
| | | glycin e(G) or a negatively charged amino acid |
| 36 | linker amino acid | (GₓS)_{y}, wherein x is 1-10 and y is 1-10 |
| | sequence | |
| 37 | linker amino acid | GGGGSGGGGSGGGGS |
| | sequence | |
| 38 | linker amino acid | GSTSGGGSGGGSGGGGSS |
| | sequence | |
| 39 | Human TGFβR1 | LQCFCHLCTKDNFTCVTDGLCFVSVTETTDKVIHNSMCIAEIDLIPRD |
| | extracellular | RPFVCAPSSKTGSVTTTYCCNQDHCNKIELPTTVKSSPGLGPVE |
| | domain | |
| 40 | Human TGFβR1 | LAAVIAGPVCFVCISLMLMVYI |
| | transmembrane | |
| | domain | |
| 41 | Human TGFβR2 | TIPPHVQKSVNNDMIVTDNNGAVKFPQLCKFCDVRFSTCDNQKSC |
| | extracellular | MSNCSITSICEKPQEVCVAVWRKNDENITLETVCHDPKLPYHDFILED |
| | domain | AASPKCIMKEKKKPGETFFMCSCSSDECNDNIIFSEEYNTSNPD |
| 42 | Human TGFβR2 | LLLVIFQVTGISLIPPLGVAISVILIFYCY |
| | transmembrane | |
| | domain | |
| 43 | Human IL-2Rβ | IPWLGHLLVGLSGAFGFIILVYLLI |
| | transmembrane | |
| | domain | |
| 44 | Human IL-2Rβ | NCRNTGPWLKKVLKCNTPDPSKFFSQLSSEHGGDVQKWLSSPFPSS |
| | cytoplasmic/sign | SFSPGGLAPEISPLEVLERDKVTQLLLQQDKVPEPASLSSNHSLTSCFT |
| | aling domain | NQGYFFFHLPDALEIEACQVYFTYDPYSEEDPDEGVAGAPTGSSPQP |
| | | LQPLSGEDDAYCTFPSRDDLLLFSPSLLGGPSPPSTAPGGSGAGEER |
| | | MPPSLQERVPRDWDPQPLGPPTPGVPDLVDFQPPPELVLREAGEE |
| | | VPDAGPREGVSFPWSRPPGQGEFRALNARLPLNTDAYLSLQELQG |
| | | QDPTHLV |
| 45 | Human IL-2Rγ | PFLFALEAVVISVGSMGLIISLLCVYFWL |
| | transmembrane | |
| | domain | |
| 46 | Human IL-2Rγ | ERTMPRIPTLKNLEDLVTEYHGNFSAWSGVSKGLAESLQPDYSERLC |
| | cytoplasmic/sign | LVSEIPPKGGALGEGPGASPCNQHSPYWAPPCYTLKPET |
| | aling domain | |
| 47 | Sequence | SSPGLGPVEL |
| | comprising | |
| | TGFBR1 partial | |
| | extracellular and | |
| | domain and | |
| | transmembrane | |
| | domain amino | |
| | acids | |
| 48 | Sequence | IFSEEYNTSNPDLL |
| | comprising | |
| | TGFβR1 partial | |
| | extracellular and | |
| | domain and | |
| | transmembrane | |
| | domain amino | |
| | acids | |
| 49 | sgTGFβRI guide | CATACAAACGGCCTATCTCG |
| | RNA | |
| 50 | sgTGFβRII guide | TCACCCGACTTCTGAACGTG |
| | RNA | |
| 51 | TGFBβI fwd | AGTGTTTCTGCCACCTCTGT |
| | genomic primer | |
| 52 | TGFBβI rev | TGCCTCTAAACGGAATGAGC |
| | genomic primer | |
| 53 | Furin cleavage | RXXR |
| | motif | |
| 54 | Furin cleavage | RAKR |
| | sequence | |
| 55 | Furin cleavage | RARR |
| | sequence | |
| 56 | TGFβRI | LAAVIAGPVCFVCISLMLMVYICHN |
| | transmembrane | |
| | sequence with | |
| | 3-amino acid | |
| | intracellular | |
| | overhang (CHN) | |
| 57 | sgTGFBRII guide | CTAGTCATATTTCAAGTGAC |
| | RNA | |
| 58 | MsIn-specific TCR | MTLKMDSSPGFVAVILLILGRTHGDSVTQTEGPVTVSESESLIINCTYS |
| | with murinized | ATSIAYPNLFWYVRYPGEGLQLLLKVITAGQKGSSRGFEATYNKETTS |
| | TGFbRI-IL2b- | FHLQKASVQESDSAVYYCALGLDYANKMIFGLGTILRVRPHIQNPEP |
| | furin-GSG-P2A- | AVYQLKDPRSQDSTLCLFTDFDSQINVPKTMESGTFITDKTVLDMKA |
| | TGFbRII-IL2g, | MDSKSNGAIAWSNQTSFTCQDIFKETNATYPSSDVPCDATLTEKSFE |
| | with signal | TDMNLNFQNLSVMGLRILLLKVAGFNLLMTLRLWSSRAKRSGSGAT |
| | peptides | NFSLLKQAGDVEENPGPMEAAAAAPRRPQLLIVLVAAATLLPGAKA |
| | | LQCFCHLCTKDNFTCETDGLCFVSVTETTDKVIHNSMCIAEIDLIPRD |
| | | RPFVCAPSSKTGAVTTTYCCNQDHCNKIELPTTGPFSEKQSAGLGPV |
| | | EILPMSWLRYLLLVLGCFSGFFSCVYILVKCRYLGPWLKTVLKCHIPDP |
| | | SEFFSQLSSQHGGDLQKWLSSPVPLSFFSPSGPAPEISPLEVLDGDSK |
| | | AVQLLLLQKDSAPLPSPSGHSQASCFTNQGYFFFHLPNALEIESCQVY |
| | | FTYDPCVEEEVEEDGSRLPEGSPHPPLLPLAGEQDDYCAFPPRDDLLL |
| | | FSPSLSTPNTAYGGSRAPEERSPLSLHEGLPSLASRDLMGLQRPLER |
| | | MPEGDGEGLSANSSGEQASVPEGNLHGQDQDRGQGPILTLNTDAY |
| | | LSLQELQAQDSVHLIRAKRGSGATNFSLLKQAGDVEENPGPMGRGL |
| | | LRGLWPLHIVLWTRIASTIPPHVPKSDVEMEAQKDASIHLSCNRTIHP |
| | | LKHFNSDVMASDNGGAVKLPQLCKFCDVRLSTCDNQKSCMSNCSI |
| | | TAICEKPHEVCVAVWRKNDKNITLETVCHDPKLTYHGFTLEDAASPK |
| | | CVMKEKKRAGETFFMCACNMEECNDYIIFSEEYTTSSPDPSLFALEA |
| | | VLIPVGTMGLIITLIFVYCWLERMPPIPPIKNLEDLVTEYQGNFSAWS |
| | | GVSKGLTESLQPDYSERFCHVSEIPPKGGALGEGPGGSPCSLHSPYW |
| | | PPPCYSLKPEARAKRSGSGQCTNYALLKLAGDVESNPGPMDPRLLCC |
| | | VIFCLLAATFVDTTVKQNPRYKLARVGKPVNLICSQTMNHDTMYWY |
| | | QKKPNQAPKLLLFYYDKILNREADTFEKFQSSRPNNSFCSLYIGSAGLE |
| | | YSAMYLCASSNPQDTQYFGPGTRLLVLEDLRNVTPPKVSLFEPSKAEI |
| | | ANKQKATLVCLARGFFPDHVELSWWVNGKEVHSGVSTDPQAYKES |
| | | NYSYCLSSRLRVSATFWHNPRNHFRCQVQFHGLSEEDKWPEGSPKP |
| | | VTQNISAEAWGRADCGITSASYHQGVLSATILYEILLGKATLYAVLVS |
| | | GLVLMAMVKKKNS |
| 59 | MsIn-specific TCR | MTLKMDSSPGFVAVILLILGRTHGDSVTQTEGPVTVSESESLIINCTYS |
| | with murinized | ATSIAYPNLFWYVRYPGEGLQLLLKVITAGQKGSSRGFEATYNKETTS |
| | TGFbRII-IL2b- | FHLQKASVQESDSAVYYCALGLDYANKMIFGLGTILRVRPHIQNPEP |
| | furin-GSG-P2A- | AVYQLKDPRSQDSTLCLFTDFDSQINVPKTMESGTFITDKTVLDMKA |
| | TGFbRI-IL2g, with | MDSKSNGAIAWSNQTSFTCQDIFKETNATYPSSDVPCDATLTEKSFE |
| | signal peptides | TDMNLNFQNLSVMGLRILLLKVAGFNLLMTLRLWSSRAKRSGSGAT |
| | | NFSLLKQAGDVEENPGPMGRGLLRGLWPLHIVLWTRIASTIPPHVP |
| | | KSDVEMEAQKDASIHLSCNRTIHPLKHFNSDVMASDNGGAVKLPQL |
| | | CKFCDVRLSTCDNQKSCMSNCSITAICEKPHEVCVAVWRKNDKNITL |
| | | ETVCHDPKLTYHGFTLEDAASPKCVMKEKKRAGETFFMCACNMEE |
| | | CNDYIIFSEEYTTSSPDLLLVIIQVTGVSLLPPLGIAIAVIIIFYCYKCRYLG |
| | | PWLKTVLKCHIPDPSEFFSQLSSQHGGDLQKWLSSPVPLSFFSPSGP |
| | | APEISPLEVLDGDSKAVQLLLLQKDSAPLPSPSGHSQASCFTNQGYFF |
| | | FHLPNALEIESCQVYFTYDPCVEEEVEEDGSRLPEGSPHPPLLPLAGE |
| | | QDDYCAFPPRDDLLLFSPSLSTPNTAYGGSRAPEERSPLSLHEGLPSL |
| | | ASRDLMGLQRPLERMPEGDGEGLSANSSGEQASVPEGNLHGQDQ |
| | | DRGQGPILTLNTDAYLSLQELQAQDSVHLIRAKRGSGATNFSLLKQA |
| | | GDVEENPGPMEÅAAAAPRRPQLLIVLVAÅATLLPGAKALQCFCHLC |
| | | TKDNFTCETDGLCFVSVTETTDKVIHNSMCIAEIDLIPRDRPFVCAPSS |
| | | KTGAVTTTYCCNQDHCNKIELPTTGPFSEKQSAGLGPVELAAVIAGP |
| | | VCFVCIALMLMVYIERMPPIPPIKNLEDLVTEYQGNFSAWSGVSKGL |
| | | TESLQPDYSERFCHVSEIPPKGGALGEGPGGSPCSLHSPYWPPPCYSL |
| | | KPEARAKRSGSGQCTNYALLKLAGDVESNPGPMDPRLLCCVIFCLLA |
| | | ATFVDTTVKQNPRYKLARVGKPVNLICSQTMNHDTMYWYQKKPN |
| | | QAPKLLLFYYDKILNREADTFEKFQSSRPNNSFCSLYIGSAGLEYSAMY |
| | | LCASSNPQDTQYFGPGTRLLVLEDLRNVTPPKVSLFEPSKAEIANKQK |
| | | ATLVCLARGFFPDHVELSWWVNGKEVHSGVSTDPQAYKESNYSYCL |
| | | SSRLRVSATFWHNPRNHFRCQVQFHGLSEEDKWPEGSPKPVTQNI |
| | | SAEAWGRADCGITSASYHQGVLSATILYEILLGKATLYAVLVSGLVLM |
| | | AMVKKKNS |
| 60 | TGFβRII | LLLVIFQVTGISLLPPLGVAISVIIIFYCYRVN |
| | transmembrane | |
| | sequence with 3- | |
| | amino acid | |
| | intracellular | |
| | overhang (RVN) | |

### EXAMPLES

### Example 1

### Design and Testing of Human Tgfbr/Il-2R Chimeric Proteins

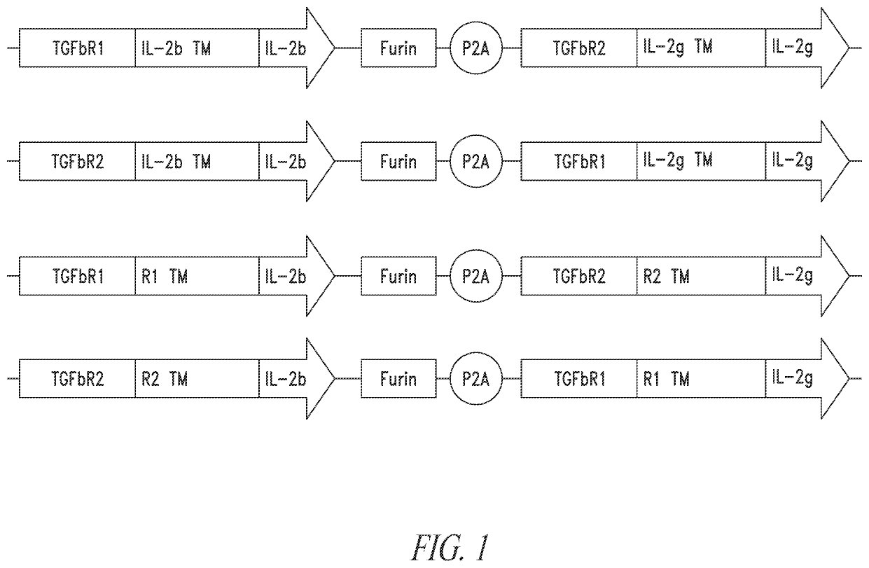

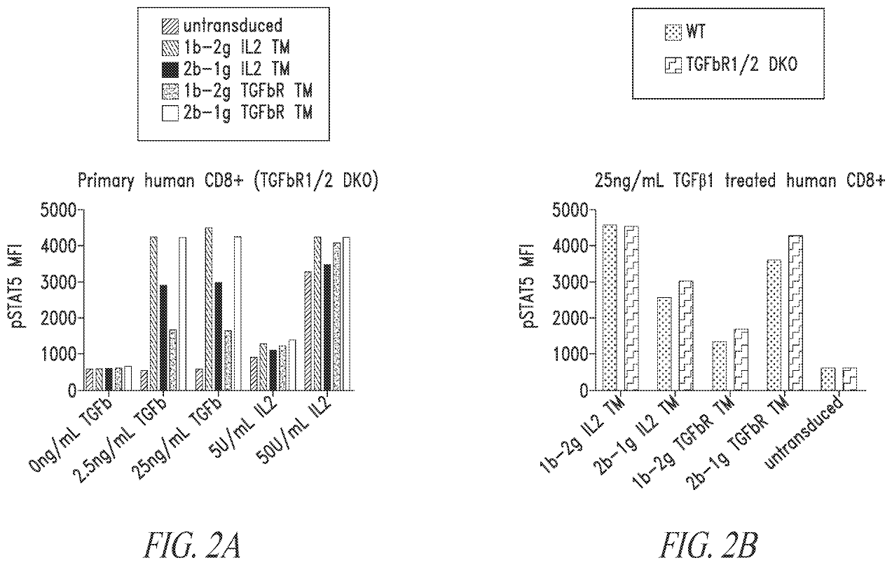

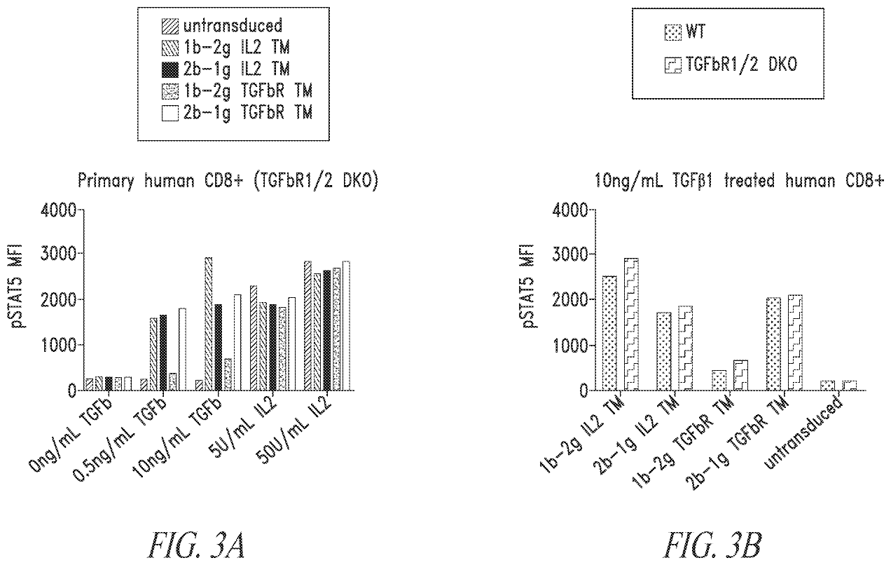

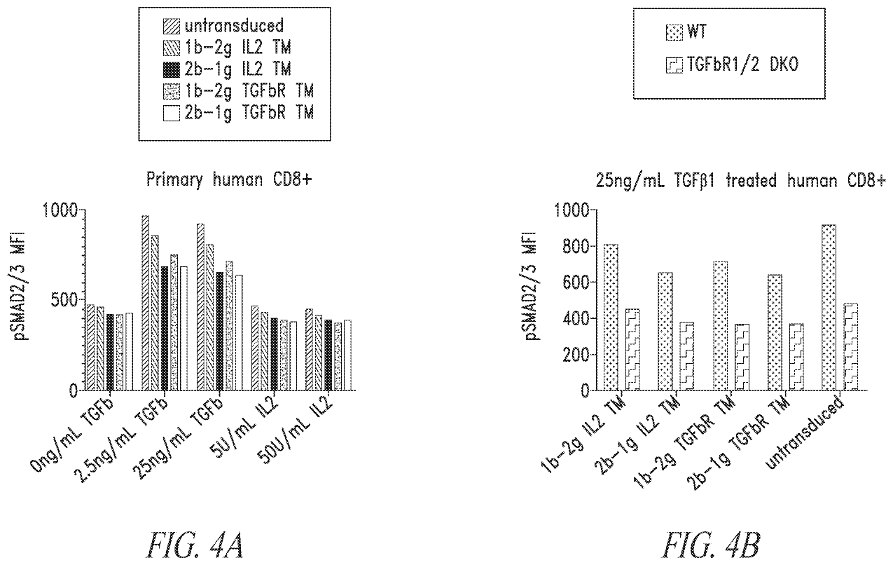

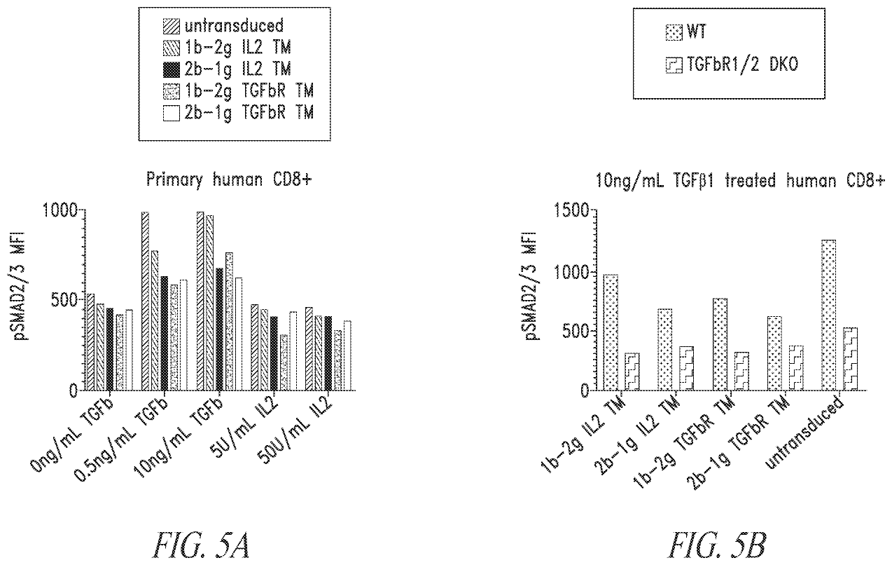

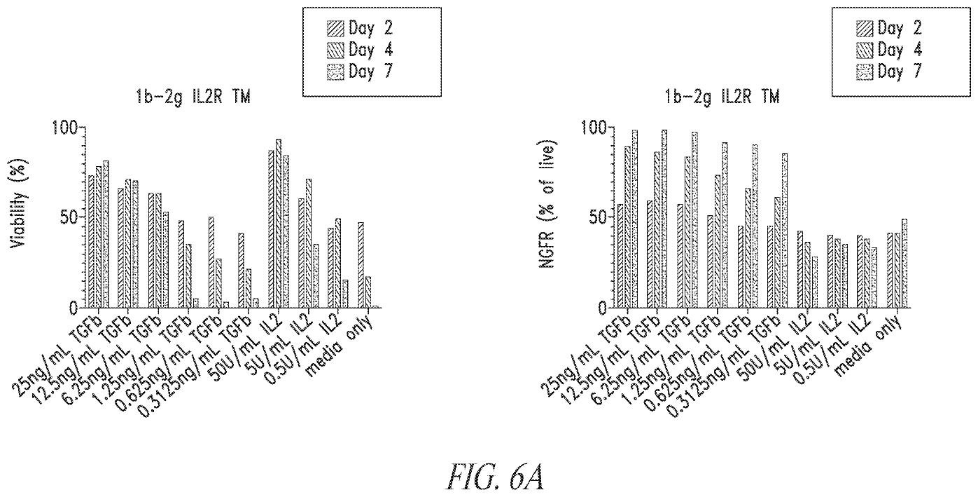

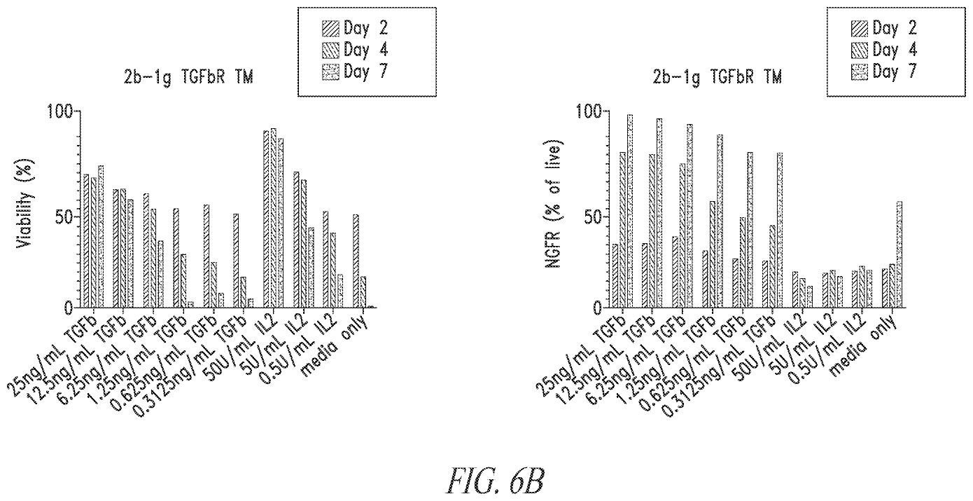

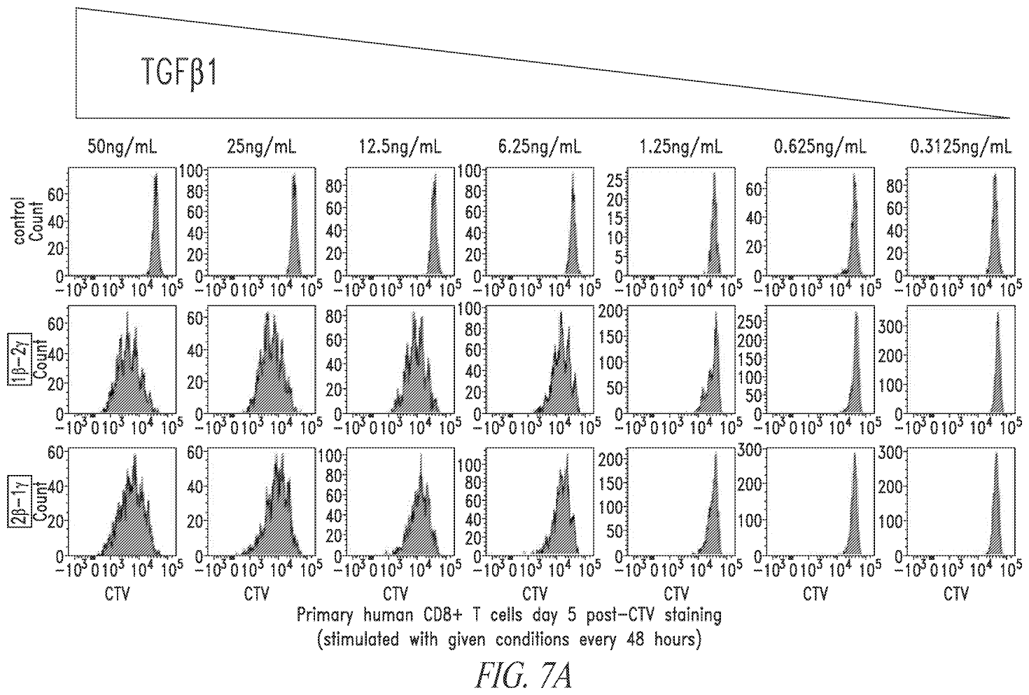

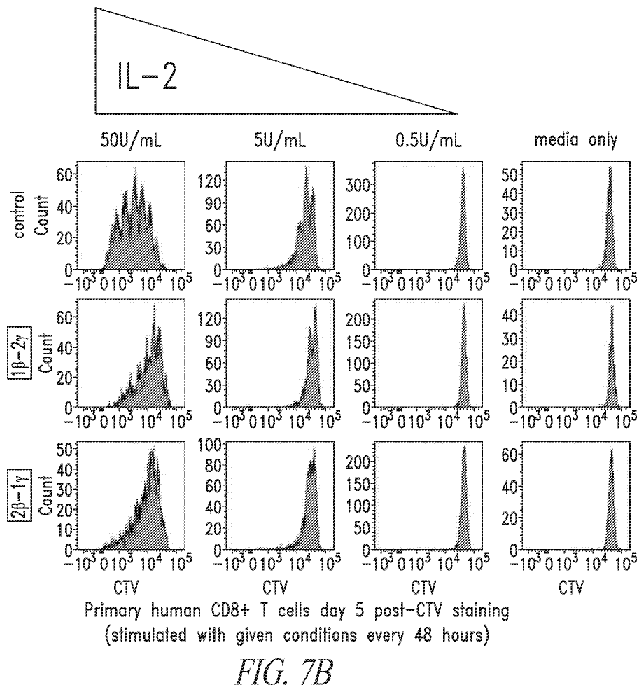

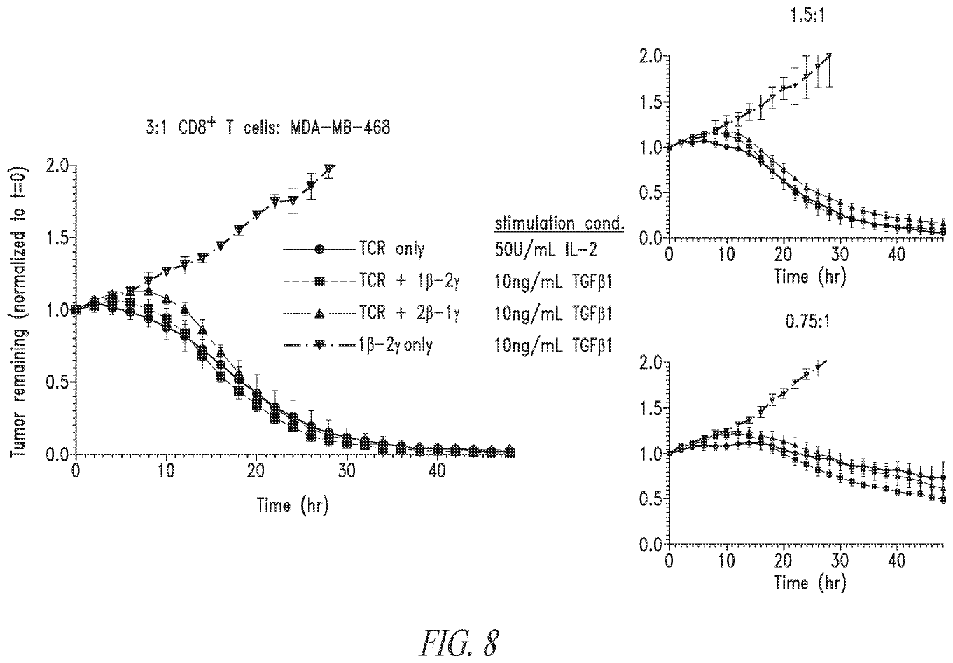

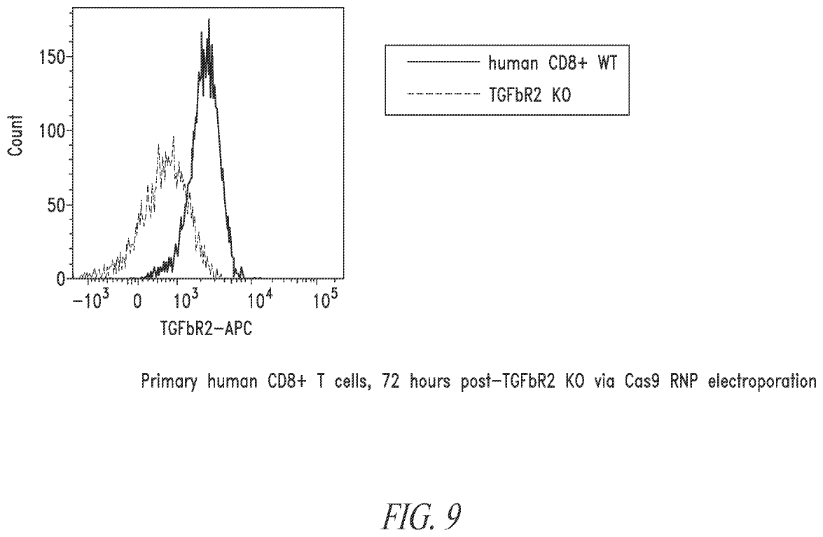

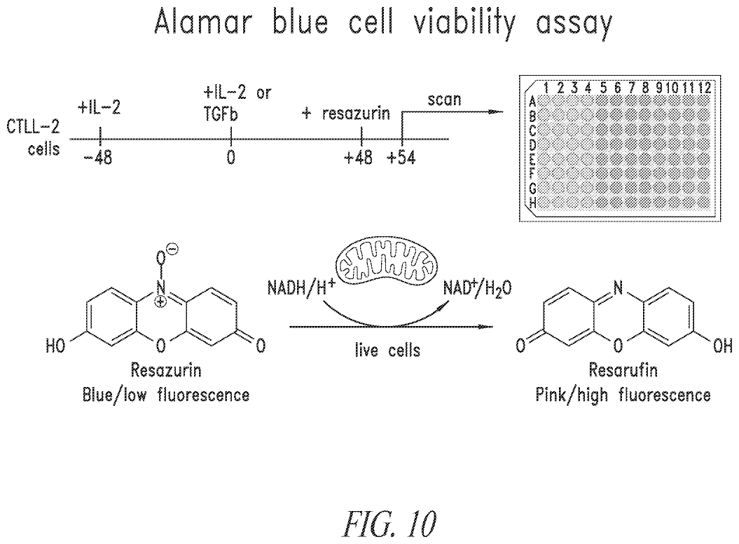

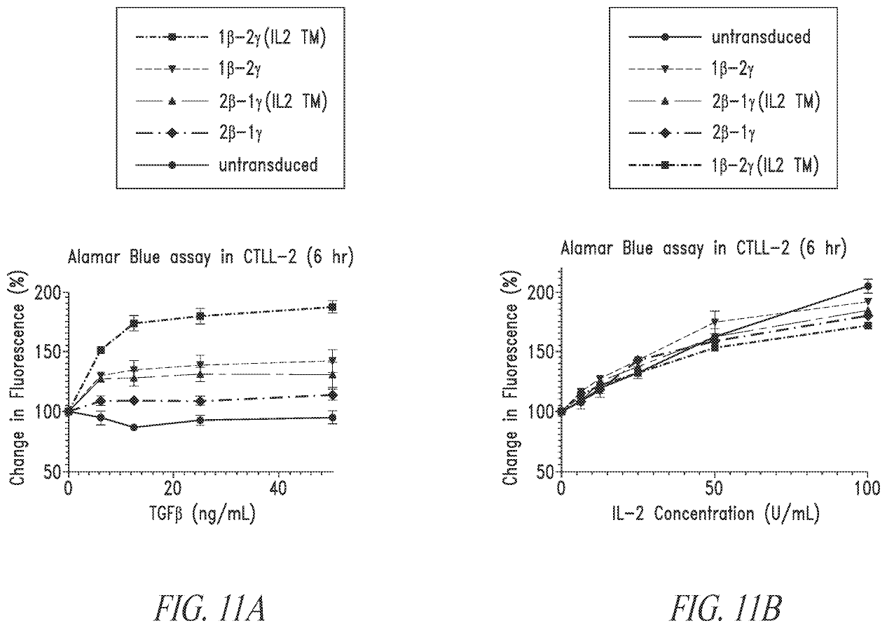

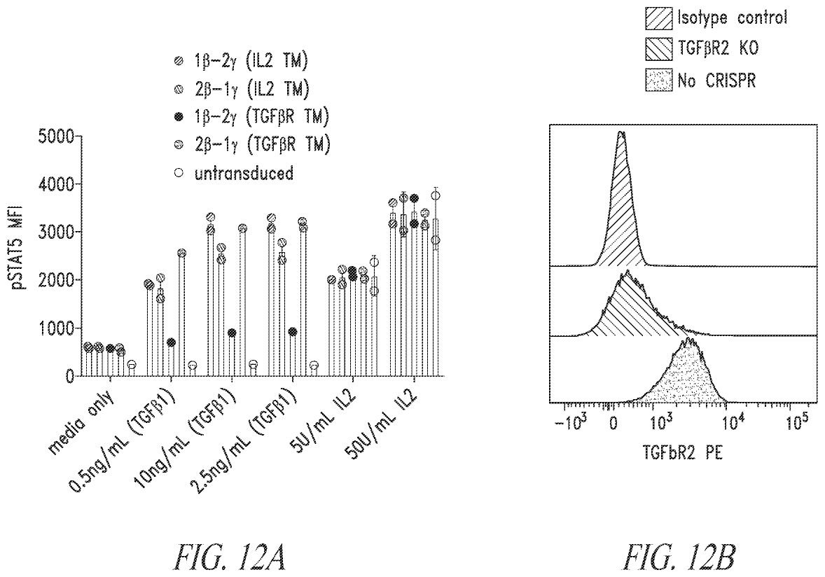

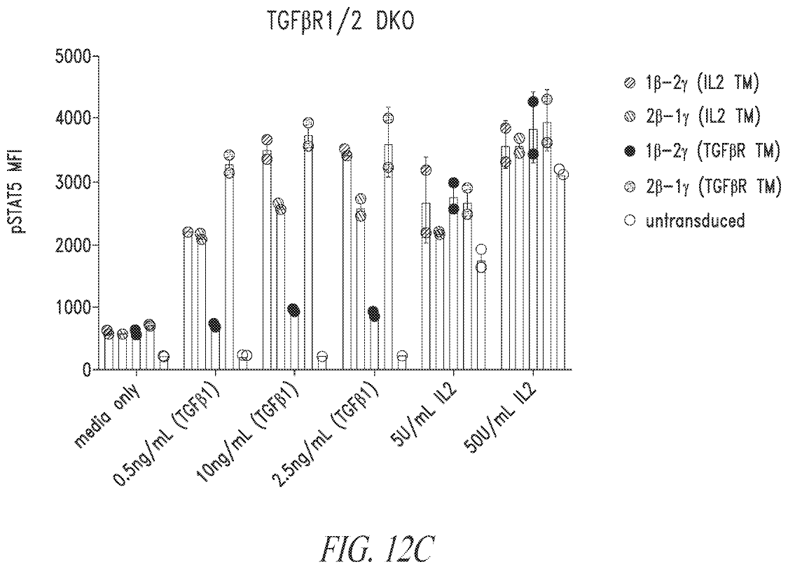

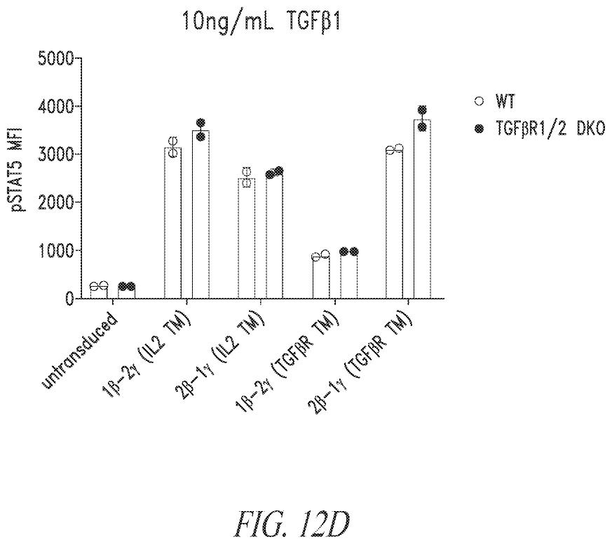

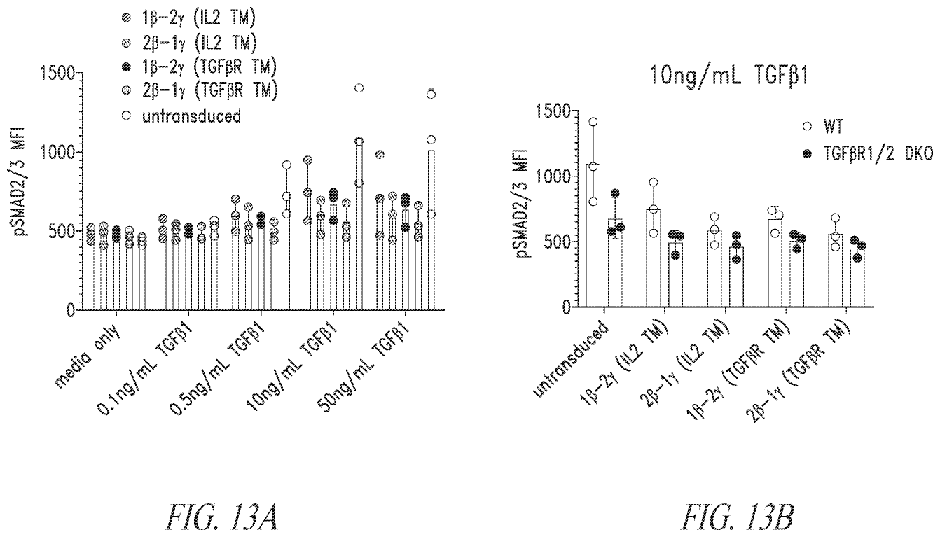

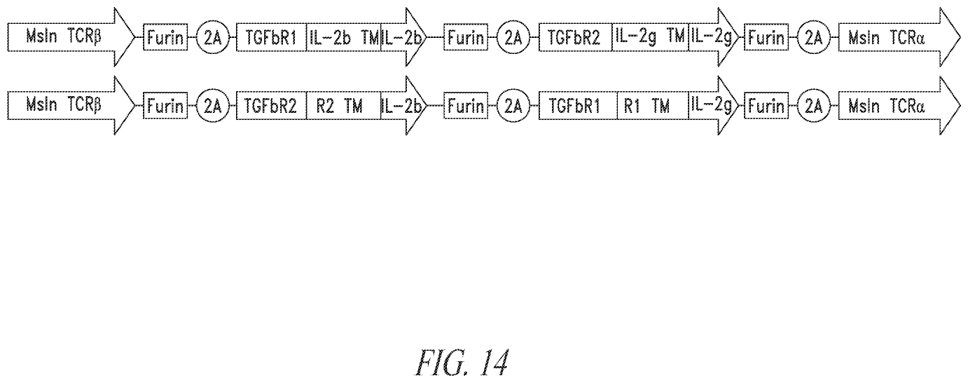

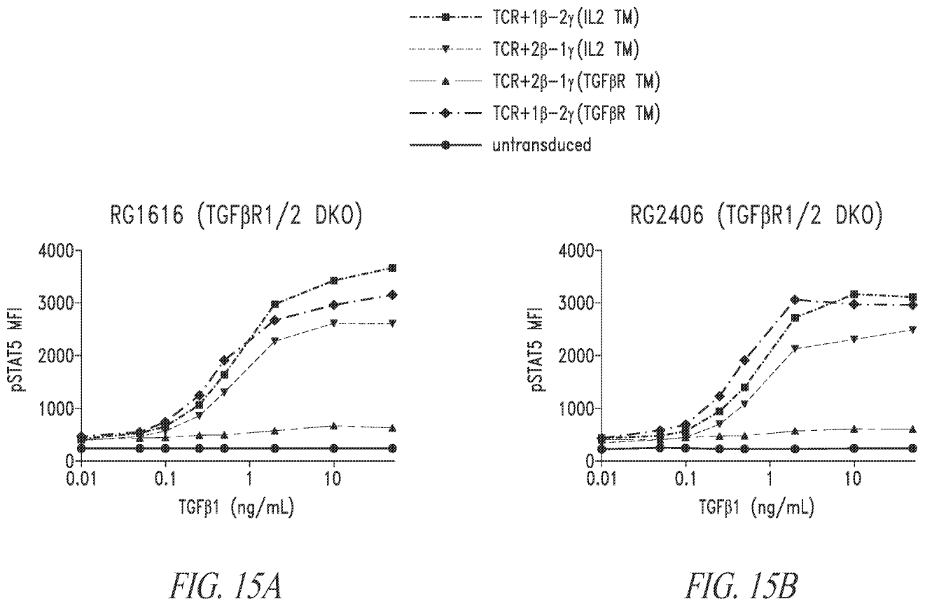

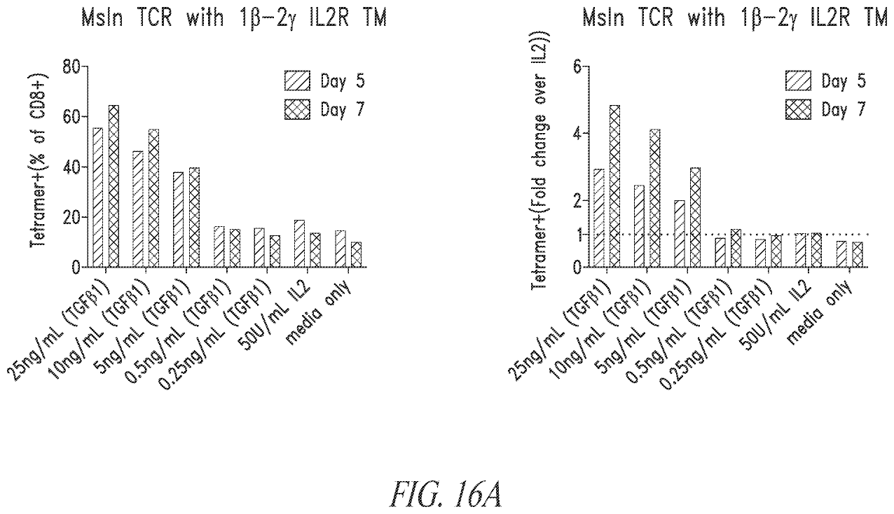

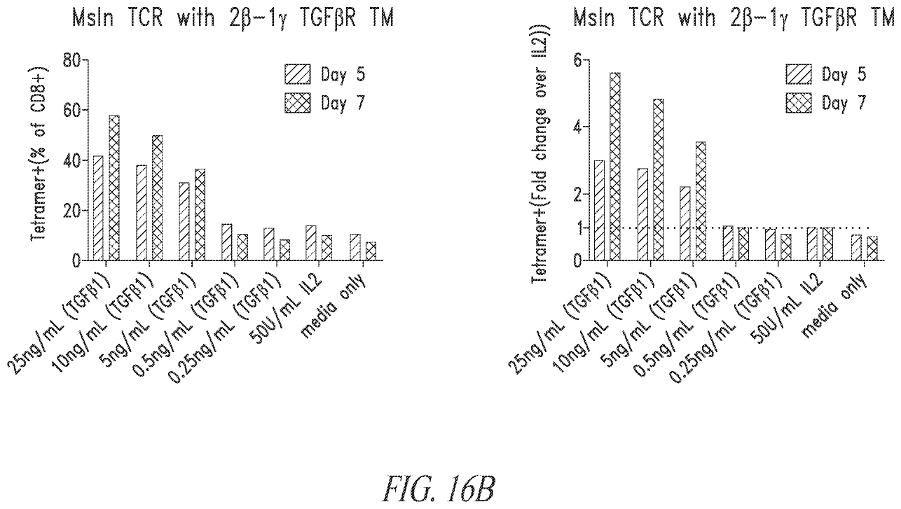

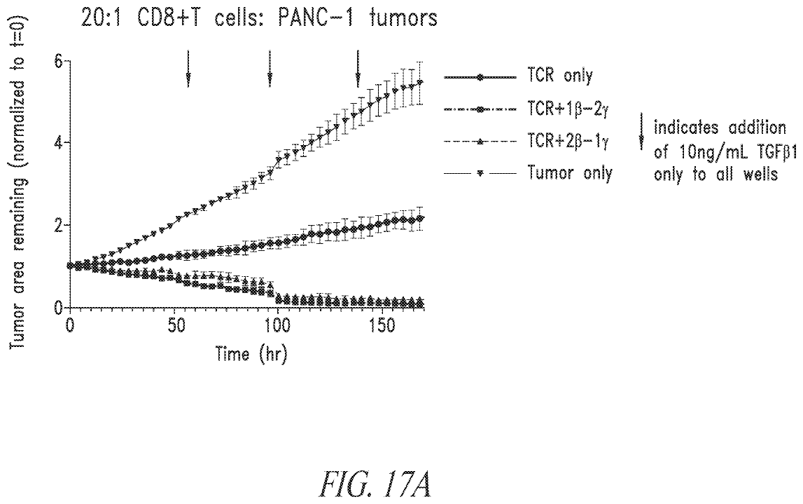

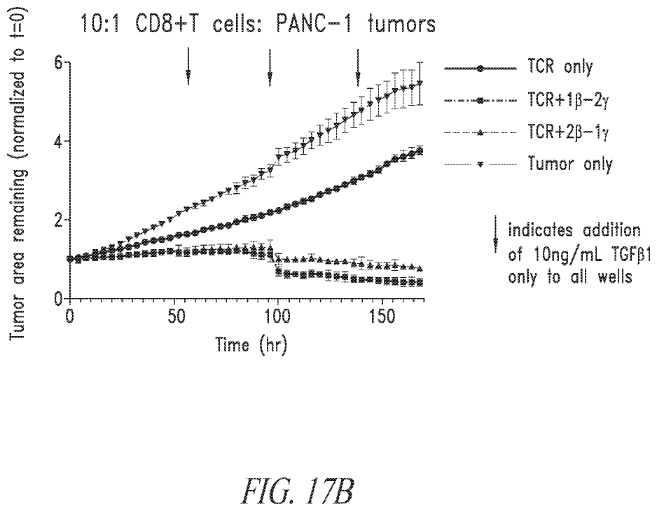

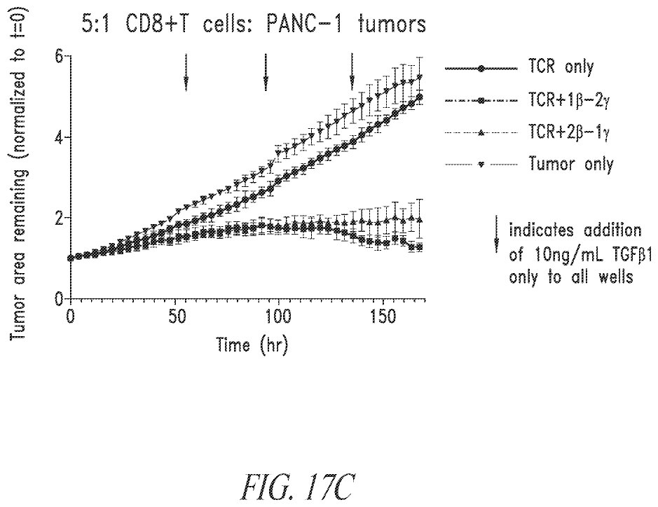

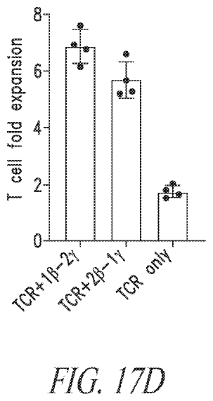

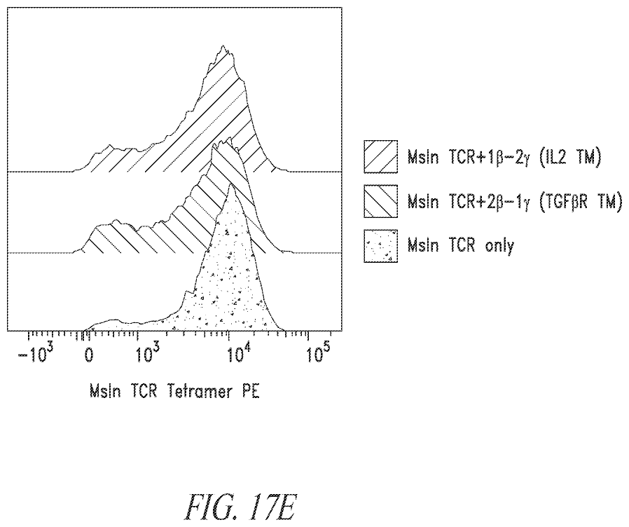

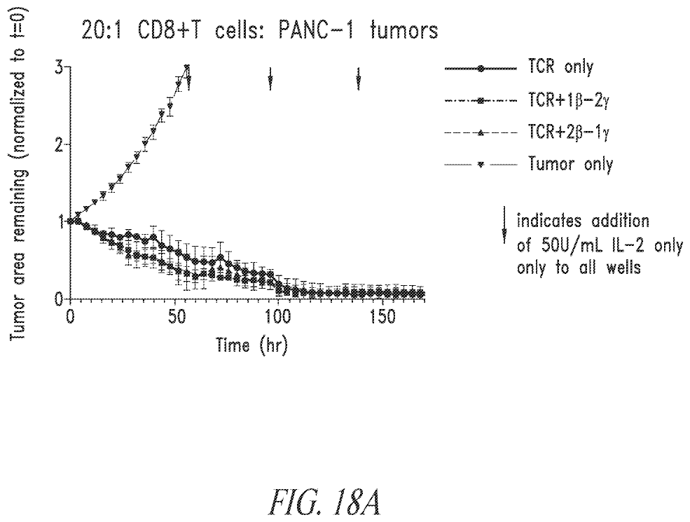

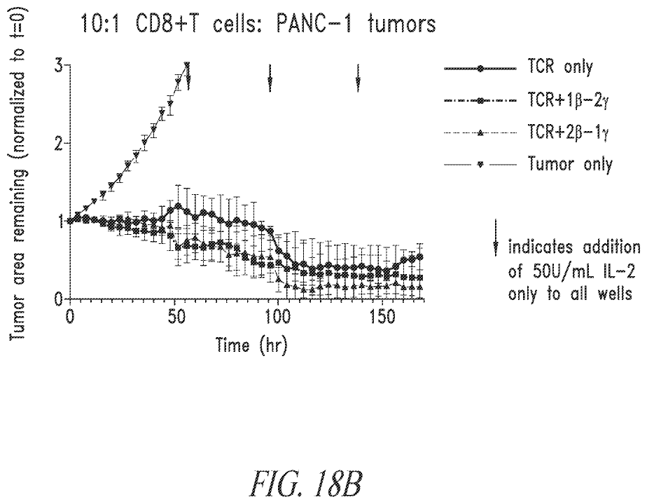

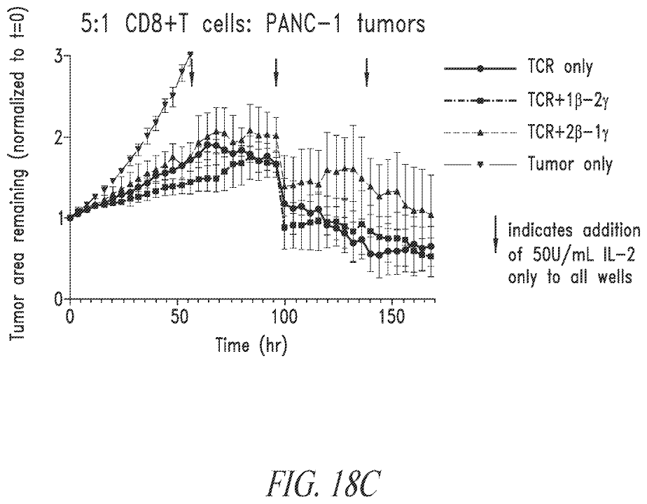

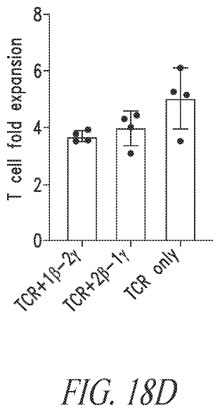

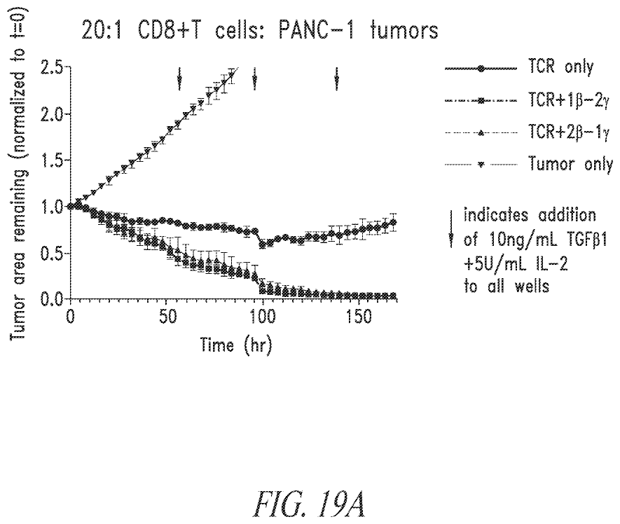

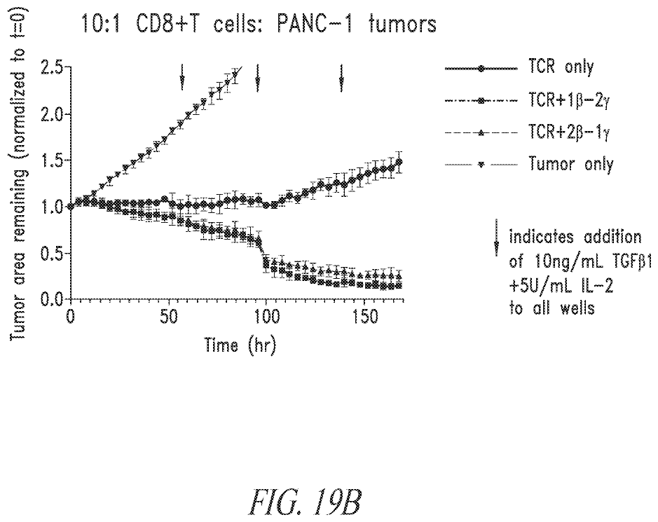

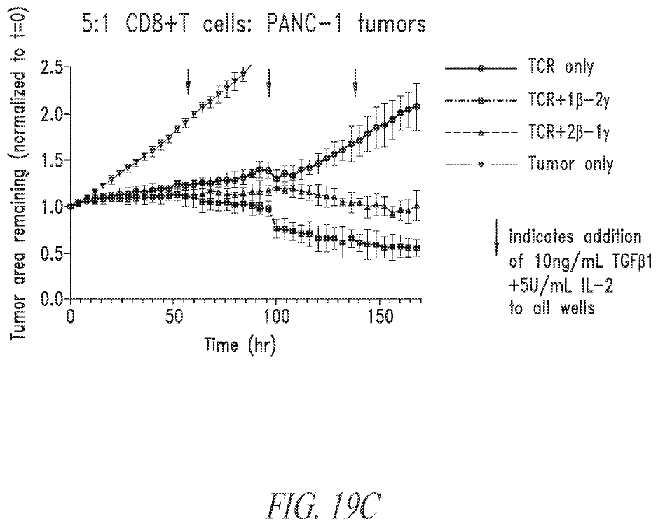

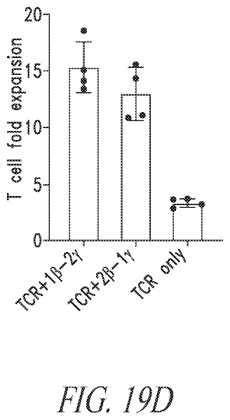

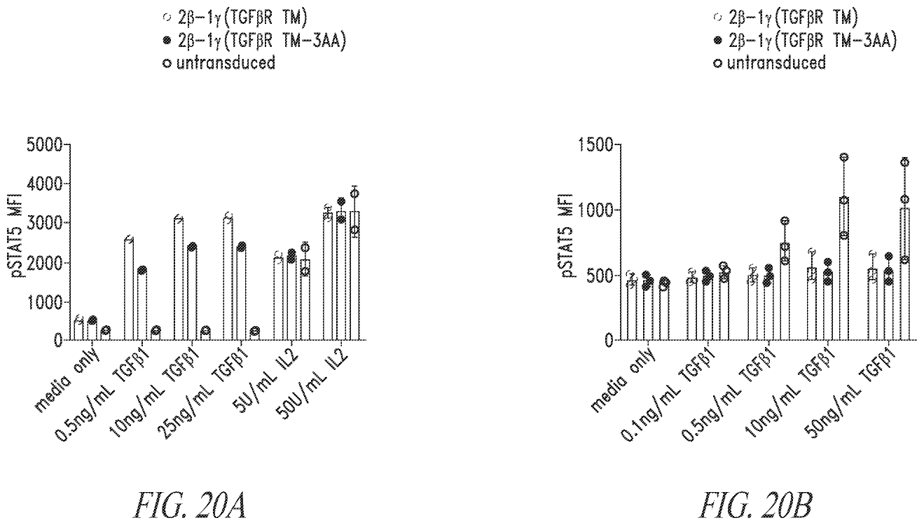

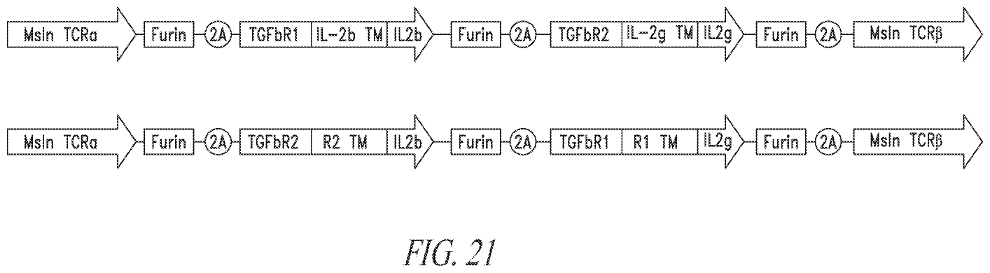

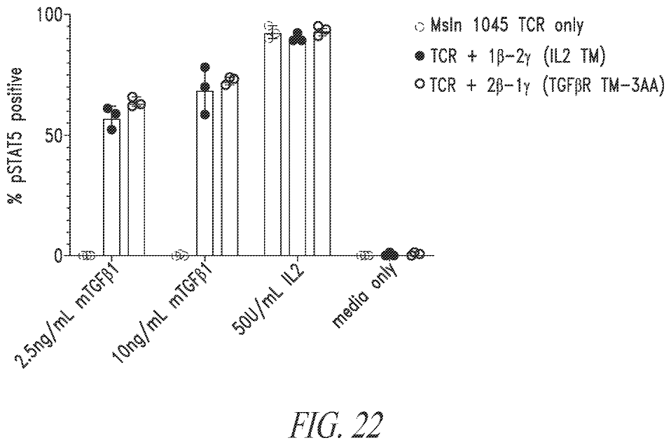

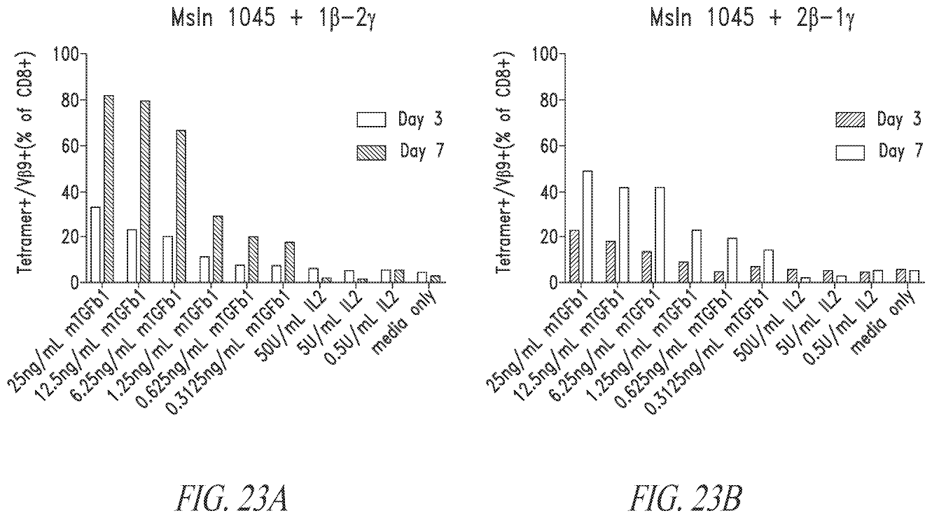

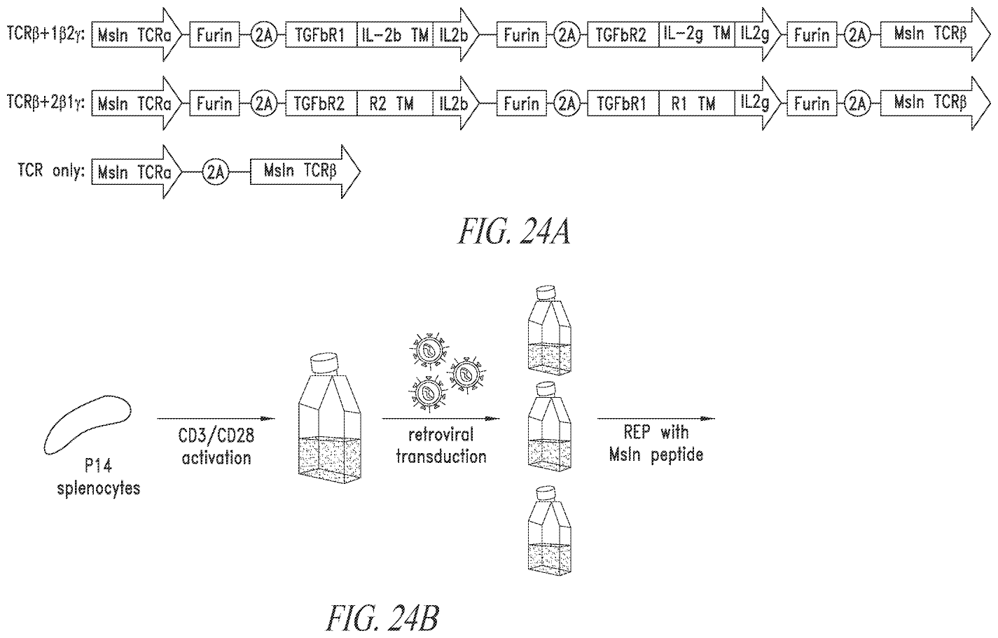

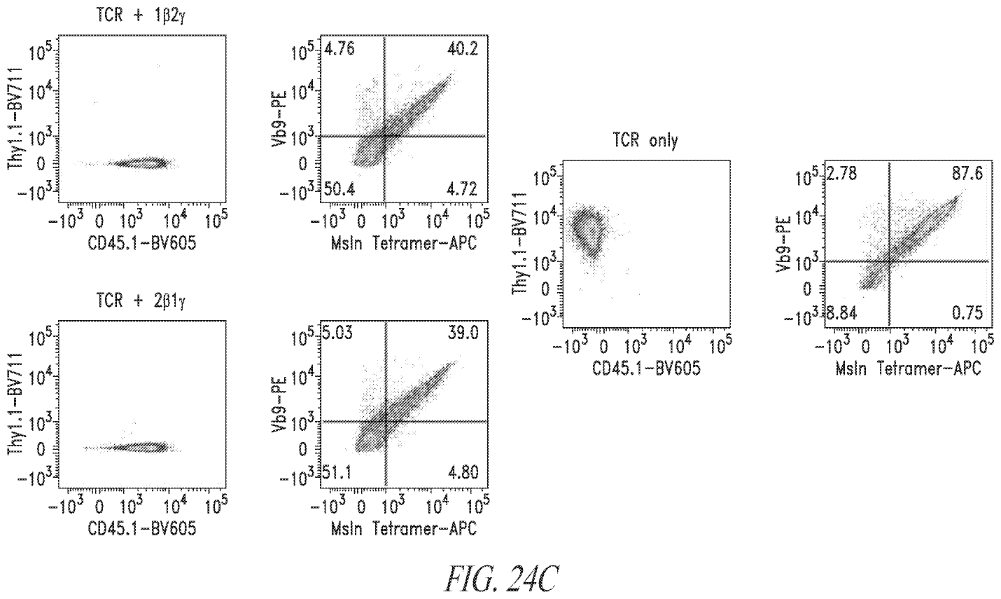

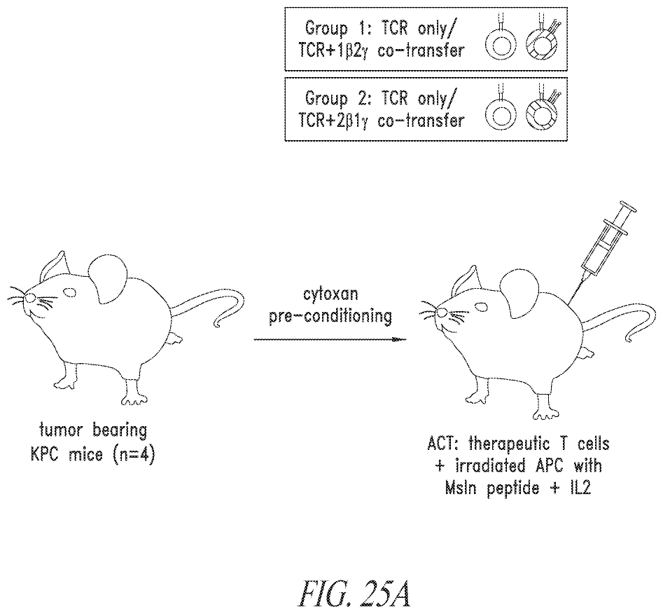

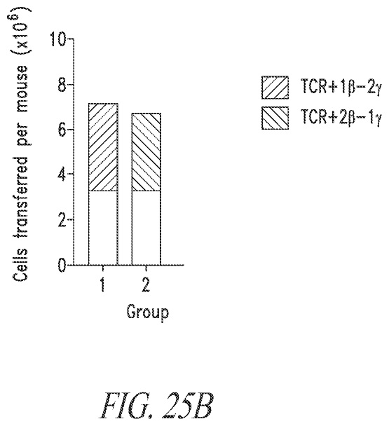

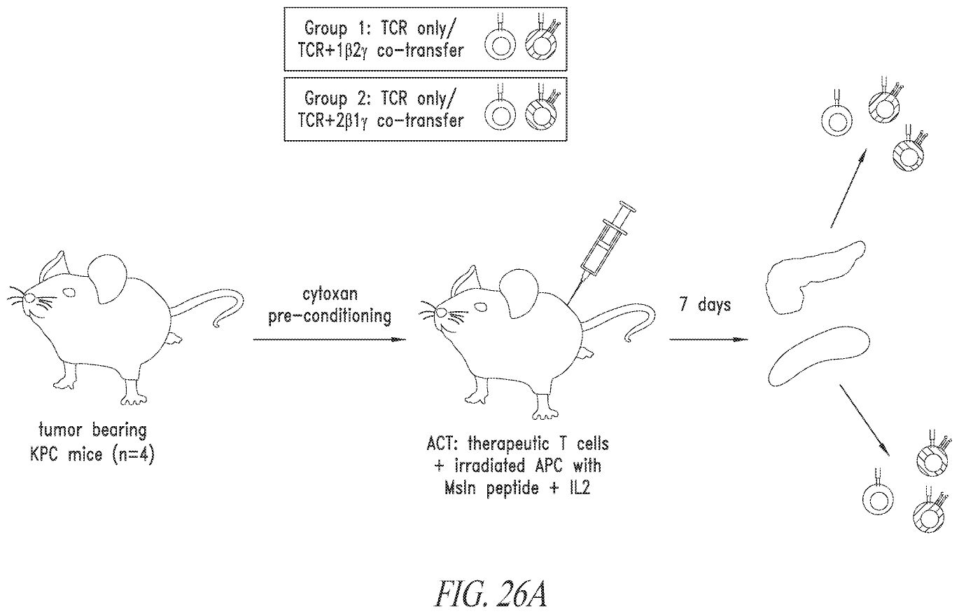

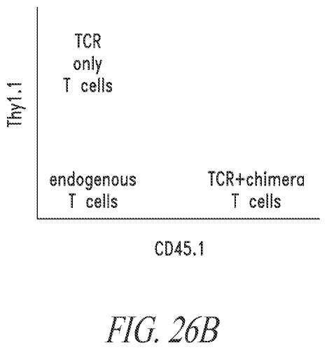

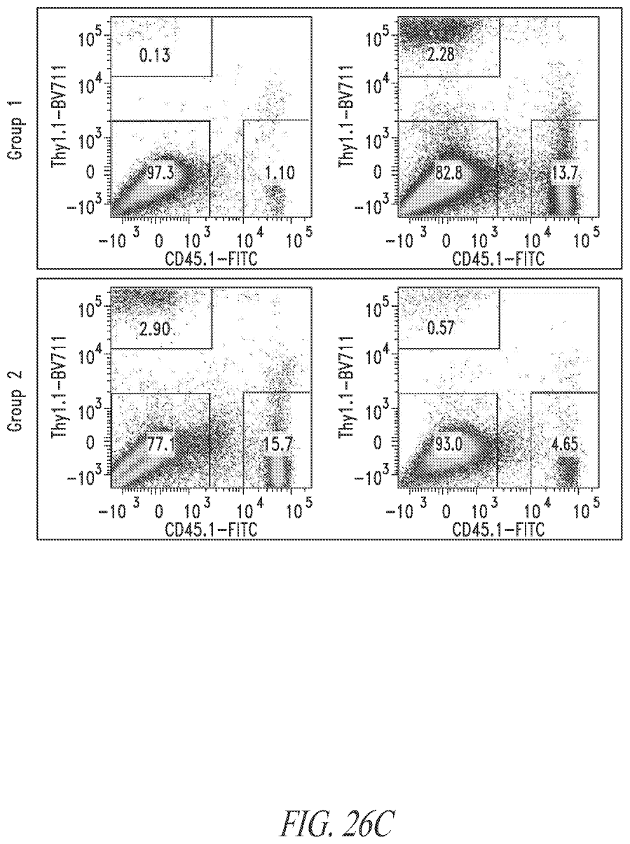

Various chimeric constructs were designed, constructed, and tested, as shown and described for FIGS. 1-20B. The data showed, for example, that: TGFβR/IL-2R chimeras repurpose TGFβ1 to transmit an IL-2 signal via pSTAT5; TGFβR/IL-2R chimeras reduce endogenous TGFβRI/II signaling via pSMAD2/3; culturing chimera-transduced T cells in TGFβ1 selects for (chimera-encoding) transgene expression; TGFβ1 promotes cell division in T cells expressing TGFβR/IL-2R chimeras; TGFβ1 dose response curve shows differences in pSTAT5 signaling mediated by TGFβR/IL-2R chimeras with a mesothelin-specific TCR; culturing TCR-transduced/chimera-transduced human T cells in TGFβ1 selects for mesothelin-HLA tetramer expression; co-expressing a mesothelin-specific TCR with TGFβR/IL-2R chimeras supports proliferation and tumor killing with TGFβ1 stimulation alone; cytotoxicity of T cells expressing the mesothelin-specific TCR and TGFβR/IL-2R chimeras is similar to that of T cells expressing the TCR only with conventional IL-2 stimulation; proliferation and cytotoxicity of T cells expressing the mesothelin-specific TCR with chimeras is enhanced by combination treatment of 10 ng/mL TGFβ1 and 5 U/mL IL-2; and addition of 3 amino acids from TGFβRI on the intracellular side of the transmembrane component of a chimeric protein promotes IL-2R signaling.

### Example 2

### Materials and Methods for Example 1

### Generation of Chimeric Constructs

Human TGFbR/IL2R chimera DNA was synthesized as gene fragments by Twist Biosciences and cloned into the pRRLSIN lentiviral backbone by Gibson Assembly using the NEBuilder HiFi DNA Assembly Kit (New England Biosciences; E2621L). Multicistronic constructs were generated by incorporation of self-cleaving viral 2A peptides, with a furin protease cleavage site 5′ of the GSG linker-2A sequence to facilitate trimming of the 2A peptide fragment from the C terminus of the polypeptide in the 5′ position. A 2A sequence followed by truncated human NGFR was added to select constructs as a transduction marker. A 20 base pair stretch of the TGFbRI sequence was codon optimized to prevent sgRNA annealing and thus enable CRISPR-Cas9 mediated knockout of the endogenous, but not transgenic, TGFbRI. The transgenic sequence for TGFbRII did not need to be codon optimized, as the sgRNA binds the endogenous sequence at an exon-intron junction. All constructs generated for these studies were sequence verified by Sanger sequencing performed by the Genomics Core Facility at the Fred Hutchinson Cancer Research Center.

### Lentiviral Packaging and Transduction

Lentivirus was generated by transfecting HEK-293T cells with the lentiviral packaging plasmids pMDLg, pMDG2, pRSV, and the transfer plasmid containing the MSCV U3 promoter and transgene of interest flanked by long terminal repeat sequences. TransIT Lenti (Mirus Bio; MIR 6603) transfection reagent was used at a ratio of 3:1 (uL TransIT:ug DNA) with a total of 2 ug DNA per well in a 6 well plate. The media was changed 24 hours post-transfection to match the media of the cells that will be transduced. After 48 hours, lentiviral supernatant was collected and filtered through a 0.45 um filter to separate out packaging cells. The desired cell population to be transduced (i.e. activated human CD8⁺ T cells) was counted and resuspended at 0.5×10⁶ cells/100 uL in human T cell media and added to 2 mL filtered lentiviral supernatant. Each well was supplemented with 2 uL polybrene and centrifuged at 2500 rpm, 30 C, for 90 minutes to enhance transduction of T cells. After centrifugation, T cells were supplemented with IL-2 to a final concentration of 50 U/mL and allowed to recover in the 37 C incubator for at least 72 hours before measuring transduction efficiency.

### Isolation of Primary Human CD8⁺ T Cells

Human healthy donor peripheral blood Leukopak apheresis products were purchased from STEMCELL Technologies (70500). Lymphocytes were washed three times with PBS supplemented with 0.5 mM EDTA, and once in RPMI 1640 base media. Total PBMCs were counted by dilution in Turk's solution. PBMCs were adjusted to a concentration of 5×10⁶ cells/mL in PBS containing 2% fetal bovine serum and 1 mM EDTA. CD8⁺ T cells were isolated using the EasySep Human CD8 T cell Isolation Kit (STEMCELL Technologies; 17953) according to the manufacturer's recommendations. T cells were checked for purity by flow cytometric analysis for CD8 as described and found to be >98% pure.

### Activation of Primary Human CD8+ T Cells

Human CD8+ T cells isolated as described above were suspended to a final concentration of 1×10⁶ in human T cell media containing: XVIVO-15 with gentamicin and phenol red base media (Lonza BioWhittaker, BW04-418Q) supplemented with 5% human serum (BloodWorks Northwest), 50 uM beta-mercaptoethanol, and 10 mM N-acetyl-L-cysteine (Sigma, A9165-25G). TCR stimulation was initiated by addition of CD3/CD28 DynaBeads at a concentration of 1 bead/cell (ThermoFisher; 11131D), 250 U/mL human IL-2 (ProLeukin), 5 ng/mL human IL-7, and 5 ng/mL human IL-15. T cells were incubated in activation cocktail for 24 hours before transduction and the DynaBeads were removed from the T cells via magnetic separation after 48 hours.

### Tissue Culture and Reagents

Primary human CD8 T cells were cultured in the following human T cell media: XVIVO-15 with gentamicin and phenol red base media (Lonza BioWhittaker, BW04-418Q) supplemented with 5% human serum (BloodWorks Northwest), 50 uM beta-mercaptoethanol, and 10 mM N-acetyl-L-cysteine (Sigma, A9165-25G). Primary human CD8 T cells were supplemented with a final concentration of 50 U/mL IL-2 every 48 hours. MDA-MB-468 breast adenocarcinoma cells (ATCC, HTB-132) were cultured in Dulbecco's Modified Eagle Medium supplemented with 10% fetal bovine serum and 1% penicillin-streptomycin (w/v). MDA-MB-468 were maintained at <80% confluence and passaged by dissociation with 0.05% Trypsin-EDTA (Gibco; 25300-054). All cell lines were confirmed to be Mycoplasma negative before use in experiments.

### Flow Cytometric Analysis and Cell Sorting

Fluorophore-conjugated human antibodies were purchased through Biolegend including NGFR (ME20.4; 345112) and TGFbRII (W 11755E; 399706). The following antibodies were purchased through BD Biosciences: pSMAD2/3 (072-670; 562586) and pSTAT5 (47; 612599), CD8 (SKI, 335787). HLA-A2/WT1 tetramer was generated by the Immune Monitoring Lab at the Fred Hutchinson Cancer Research Center. The LIVE/DEAD Fixable Aqua Dead Cell Stain Kit (ThermoFisher; L34957) was used to label cells according to the manufacturer's recommendations. After antibody staining, cells were resuspended in FACS buffer (PBS supplemented with 2% fetal bovine serum (v/v) and 0.5 mM EDTA) for flow cytometric analysis. Flow cytometry data were acquired on a BD Biosciences Canto II instrument or BD Biosciences Aria II sorter for flow cytometric cell sorting using FACSDiva software. FlowJo v9 software (TreeStar, Ashland OR) was used to analyze all flow cytometry data.

### Analysis of Intracellular Signaling by Phospho-Flow Cytometry

T cells transduced with chimeric constructs were washed twice in XVIVO-15 serum-free base medium and starved of serum overnight. T cells were counted and plated in 96 well plates with 2×10⁵ cells/well. Cells were stained with LIVE/DEAD Fixable Violet Stain (ThermoFisher; L34955) according to the manufacturer's recommendations. Cells were stained for NGFR in FACS buffer and then washed and resuspended in human T cell media supplemented with the indicated amounts of TGFb1 or IL-2 and incubated for exactly 15 minutes at 37 C. From here, proceed to either pSTAT5 staining or pSMAD2/3 staining as outlined below.

For pSTAT5 staining, directly add 16% paraformaldehyde to a final concentration of 3% and incubate at room temperature for 10 minutes. Centrifuge at 1350 rpm for 4 minutes, discard media, and wash in FACS buffer. Permeabilize the cells by slowly adding 50 uL/well ice cold Phosflow Perm Buffer III (BD Biosciences; 558050) and pipette well to mix. Incubate on ice for 15-20 minutes. Wash twice in cold FACS buffer and then stain for pSTAT5 in FACS buffer at 4 C for 45 minutes, protected from light. Wash once with FACS buffer and then resuspend in FACS buffer for flow cytometric analysis.

For pSMAD2/3 staining, spin down cells, discard media, and resuspend in CytoFix/CytoPerm solution (BD Biosciences; 554714) and incubate at room temperature for 20 minutes. Wash with 1× PermWash buffer and then stain for pSMAD2/3 in 1× PermWash buffer at 4 C for 45 minutes, protected from light. Wash once with 1× PermWash buffer and once with FACS buffer. Then resuspend in FACS buffer for flow cytometric analysis.

### T Cell Proliferation Assay

T cells transduced with the indicated constructs were labeled with CellTrace Violet Cell Proliferation Kit (ThermoFisher; C34557) according to the manufacturer's recommendations. After labeling, T cells were resuspended in human T cell media containing varying concentrations of either IL-2 or TGFb1 (R&D Systems; 7754-BH). T cells were re-stimulated with either IL-2 or TGFb1 as indicated every 48 hours. After 5 days, the T cells were resuspended in FACS buffer supplemented with propidium iodide (ThermoFisher; P1304MP) for live/dead discrimination and analyzed by flow cytometry.

### IncuCyte Tumor Killing Assay

NucLight Red Lentivirus Reagent (Essen BioScience, 4625) was used to transduce MDA-MB-468 cells according to the manufacturer's recommended infection protocol. The NucLight Red positive population was sorted and checked for purity by FACS as described. For MDA-MB-468 killing experiments, tumors were suspended in human T cell media and plated in flat-bottom 96-well plates at 5,000 cells/well 16 hours before initiating the experiment to allow cells to adhere overnight. Human primary CD8+ T cells were purified, activated, and transduced with the indicated lentiviral constructs and either sorted to purity via FACS (TCR only) or selected for transgene expression by culturing in 10 ng/mL recombinant human TGFb1 (TCR+chimera constructs, chimera only construct). After 7 days in selection media, the cells were checked by flow cytometry for purity and all T cell conditions were >95% positive for transgene expression (measured via staining for the transduction marker tNGFR). Immediately before data collection, T cells were counted, resuspended in warm human T cell media, and added to 96 well plates at the indicated effector:target cell ratios based on a fixed count of 5,000 tumor cells/well. Tumor cell killing data was generated on the IncuCyte S3 automated imaging platform (Essen BioScience) configured to acquire images every 2 hours for the indicated length of each experiment. Tumor cell killing was quantified by the loss of NucLight Red signal (total red object area per well normalized to the total red object area per well at the time of the first scan). The mean (n=3 wells per condition) for each time point was plotted using GraphPad Prism software with standard deviation indicated by error bars.

### CRISPR-Cas9 Mediated Knockout of TGFbRII Via RNP Electroporation

Guide RNAs targeting human TGFbRI and TGFbRII were designed using the Benchling CRISPR gRNA Design Tool with the following sequences (5′ to 3′):

```
  (SEQ ID NO.: 49)
TGFbRI: CATACAAACGGCCTATCTCG
  (SEQ ID NO.: 50)
TGFbRII: TCACCCGACTTCTGAACGTG
For other experiments, the following TGFbRII
gRNA was used;
  (SEQ ID NO.: 57)
CTAGTCATATTTCAAGTGAC
```

Guide RNA was ordered from IDT (Alt-R CRISPR-Cas9 crRNA) and complexed with tracrRNA (Dharmacon; U-002005-20) by incubation at 1:1 molar ratio at 37 C for 30 minutes to form sgRNA. Polyglutamic acid (Sigma; P4761) was added to promote stabilization of the Cas9 RNP complex prior to addition of Cas9 protein (Berkeley MacroLab) at a molar ratio of 2:1 sgRNA:Cas9 with a final concentration of 50 pmol RNP per electroporation. RNP was incubated for 15 minutes at 37 C before addition to cells. Human CD8⁺ T cells activated as described for 48 hours were separated from activation beads via magnet and counted. Cells were centrifuged at 100×g for 10 minutes and resuspended at 1×10⁶ cells/20 uL P3 buffer (Lonza P3 Primary Cell 96 Well Kit; V4SP-3096). Pre-formed RNP complex was added to the cell solution, transferred to a single well, and electroporated via a Lonza 4D Nucleofector using the pulse code EO-115. Immediately after electroporation, 80 μL of pre-warmed human T cell media was added to each well and cells were allowed to recover in the 37 C incubator for 15 minutes. Cells were then transferred to 12 well plates and allowed to rest for 72 hours before analyzing knockout efficiency by flow cytometry (TGFbRII) or harvesting and amplification of genomic DNA, Sanger sequencing, and analysis by inference of CRISPR edits (TGFbRI) using the Synthego ICE tool (Hsiau et al. bioRxiv 2018 DOI:10.1101/251082). The following primers were used to amplify the region of interest from the genomic template DNA:

(SEQ ID NO.: 51)
TGFbRI fwd: AGTGTTTCTGCCACCTCTGT
(SEQ ID NO.: 52)
TGFbRI rev: TGCCTCTAAACGGAATGAGC

### Example 3

### Design and Testing of Murine Tgfbr/Il-2R Chimeric Proteins

Murine constructs were designed and tested using methods similar to those described in Example 2, e.g. except where otherwise indicated for FIGS. 21-26C. The data showed, for example, that: murine TGFβR/IL-2R chimeras convert mouse TGFβ1 to an IL-2 signal via pSTAT5; culturing murine T cells in TGFβ1 selects for tetramer expression; murine therapeutic T cells expressing a murine TCR specific for mesothelin and murine chimeric proteins of the present disclosure were produced and characterized; the TCR with TGFβR/IL-2R chimeras was provided as TCR-T cell therapy for Pancreatic Ductal Adenocarcinoma (PDA) in a genetically engineered KrasLSL-G12D/+; Trp53LSL-R172H/+;p48Cre/+(KPC) mouse model; and the engineered TCR-T cells containing TGFβR/IL-2R chimeras preferentially accumulate in pancreatic tumors relative to T cells expressing the TCR only.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/222,400, filed on Jul. 15, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A fusion protein comprising:
(i) an extracellular component comprising an extracellular domain of a transforming growth factor beta receptor (TGFβR) polypeptide, or a portion or variant thereof that is capable of binding to a TGFβ polypeptide;
(ii) an intracellular component comprising an intracellular portion of an interleukin-2 receptor (IL-2R) polypeptide; and
(iii) a transmembrane component disposed between the extracellular component of (i) and the intracellular component of (ii),
wherein the fusion protein, when expressed by a host cell and bound to the TGFβ polypeptide, is capable of contributing to an IL-2 signal in the host cell.

2. The fusion protein of claim 1, wherein the TGFβR polypeptide comprises a TGFβRI polypeptide or a TGFβR2 polypeptide, and/or wherein the IL-2R polypeptide comprises an IL-2Rβ polypeptide, an IL-2Rγ polypeptide, or both.

3. (canceled)

4. The fusion protein of claim 1, wherein:
(1) (i) the extracellular component comprises an extracellular domain of a transforming growth factor beta receptor 1 (TGFβR1) polypeptide, or a portion or variant thereof that is capable of binding to a TGFβ polypeptide; and
(ii) the intracellular component comprises an intracellular portion of an interleukin-2 receptor beta (IL-2Rβ) polypeptide;
(2) (i) the extracellular component comprises an extracellular domain of a transforming growth factor beta-receptor 2 (TGFβR2) polypeptide, or a portion or variant thereof that is capable of binding to a TGFβ polypeptide; and
(ii) the intracellular component comprises an intracellular portion of an interleukin-2 receptor beta (IL-2Rβ) polypeptide;
(3) (i) the extracellular component comprises an extracellular domain of a transforming growth factor beta receptor 2 (TGFβR2) polypeptide, or a portion or variant thereof that is capable of binding to a TGFβ polypeptide; and
(ii) the intracellular component comprises an intracellular portion of an interleukin-2 receptor gamma (IL-2Rγ) polypeptide; or
(4) (i) the extracellular component comprises an extracellular domain of a transforming growth factor beta receptor 1 (TGFβR1) polypeptide, or a portion or variant thereof that is capable of binding to a TGFβ polypeptide; and
(ii) the intracellular component comprises an intracellular portion of an interleukin-2 receptor gamma (IL-2Rγ) polypeptide.

5.-7. (canceled)

8. The fusion protein of claim 1, wherein the transmembrane component comprises a transmembrane domain from IL-2Rβ or from IL-2Rγ.

9. (canceled)

10. The fusion protein of claim 1, wherein
the transmembrane component comprises a transmembrane domain from TGFβR1 or a transmembrane domain from TGFβR2.

11. (canceled)

12. The fusion protein of claim 10, wherein the intracellular component of the fusion protein further comprises, disposed N-terminal to the IL-2R intracellular portion, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five, six, seven, eight, nine, ten, eleven, twelve, thirteen, fourteen, fifteen, sixteen, seventeen, eight, nineteen, or twenty consecutive N-terminal amino acid(s) from TGFβR1 intracellular domain, or a variant thereof comprising one, two, three, four, or five, optionally conservative, amino acid substitutions.

13. The fusion protein of claim 10, wherein the intracellular component of the fusion protein comprises the amino acids C-H-N disposed N-terminal to the IL-2R intracellular portion.

14. The fusion protein of claim 10, wherein the intracellular component of the fusion protein further comprises, disposed N-terminal to the IL-2R intracellular portion, the one to twenty, one to fifteen, one to ten, one to five, two to twenty, two to fifteen, two to ten, two to five, three to twenty, three to fifteen, three to ten, three to five, up to five, up to ten, up to fifteen, up to twenty, or one, two, three, four, five six, seven, eight, nine, ten, eleven, twelve, thirteen, fourteen, fifteen, sixteen, seventeen, eight, nineteen, or twenty consecutive N-terminal amino acid(s) from TGFβR2 intracellular domain, or a variant thereof comprising one, two, three, four, or five, optionally conservative, amino acid substitutions.

15. The fusion protein of claim 10, wherein the intracellular component of the fusion protein comprises the amino acids R-V-N disposed N-terminal to the IL-2R intracellular portion.

16.-19. (canceled)

20. The fusion protein of claim 1, wherein:
(1) (i) the extracellular component has at least 90%, at least 91%, at least 92%, at least 93%, at least 94%, at least 95%, at least 96%, at least 97%, at least 98%, or at least 99% identity to, or comprises or consists of, the amino acid sequence set forth in SEQ ID NO.:39 or 41;
(ii) the transmembrane component has at least 90%, at least 91%, at least 92%, at least 93%, at least 94%, at least 95%, at least 96%, at least 97%, at least 98%, or at least 99% identity to, or comprises or consists of, the amino acid sequence set forth in: SEQ ID NO.:40; SEQ ID NO.:42; SEQ ID NO.:43; or SEQ ID NO.:45; and
(iii) the intracellular component has at least 90%, at least 91%, at least 92%, at least 93%, at least 94%, at least 95%, at least 96%, at least 97%, at least 98%, or at least 99% identity to, or comprises or consists of, the amino acid sequence set forth in SEQ ID NO.:44 or 46,
and/or wherein the fusion protein comprises (i)-(iii) above and comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOS.:5, 6, 9, 10, 13, 14, 17, and 18; and/or
(2) the fusion protein comprises one or more amino acid substitutions, insertions, and/or deletions to reduce or prevent the fusion protein from forming a protein dimer with a human TGFβRI or a human TGFβR2; or
(3) the fusion protein comprises one or more amino acid substitutions, insertions, and/or deletions to reduce or prevent the fusion protein from forming a protein dimer with a human TGFβRI or a human TGFβR2 wherein the one or more amino acid substitutions, insertions, and/or deletions provide:
(i) a variant of SEQ ID NO.:47, wherein the variant comprises from one to ten amino acid insertions, or from one to ten amino acid deletions;
(ii) one, two, three, four, five, six, seven, eight, nine, ten, or more amino acids added to the N-terminus and/or to the C-terminus of SEQ ID NO.:47;
(iii) a variant of SEQ ID NO.:48, wherein the variant comprises from one to ten amino acid insertions, or from one to ten amino acid deletions;
(iv) one, two, three, four, five, six, seven, eight, nine, ten, or more added to the N-terminus and/or to the C-terminus of SEQ ID NO.:48.

21. The fusion protein of claim 1, wherein the transmembrane component comprises a transmembrane domain from the TGFβR polypeptide and the intracellular component comprises, extending from the transmembrane domain and N-terminal to the intracellular portion of the IL-2R polypeptide, a TGFβR intracellular overhang or intracellular overhang sequence, wherein:
(i) the transmembrane component comprises a TGFβR1 transmembrane domain and the intracellular component comprises, extending from the TGFβR1 transmembrane domain and N-terminal to the intracellular portion of the IL-2R polypeptide, a TGFβR1 intracellular overhang or intracellular overhang sequence; and/or
(ii) the transmembrane component comprises a TGFβR2 transmembrane domain and the intracellular component comprises, extending from the TGFβR2 transmembrane domain and N-terminal to the intracellular portion of the IL-2R polypeptide, a TGFβR2 intracellular overhang or intracellular overhang sequence.

22. The fusion protein of claim 1, wherein the transmembrane component and a portion of the intracellular component together comprise the amino acid sequence of SEQ ID NO.:56 or SEQ ID NO.:60.

23.-24. (canceled)

25. A fusion protein comprising:
(A) (i) an extracellular component comprising an extracellular domain from human TGFβRI; (ii) a transmembrane component comprising a transmembrane domain from human TGFβRI; and (iii) an intracellular component comprising a cytoplasmic/signaling domain from human IL-2Rβ;
(B) (i) an extracellular component comprising an extracellular domain from human TGFβRI; (ii) a transmembrane component comprising a transmembrane domain from human TGFβRI; and (iii) an intracellular component comprising a cytoplasmic/signaling domain from human IL-2Rβ, wherein the intracellular component further comprises a human TGFβR1 intracellular overhang or intracellular overhang sequence comprising, consisting essentially of, or consisting of the amino acid sequence C-H-N, disposed N-terminal to the human IL-2Rβ cytoplasmic/signaling domain;
(C) (i) an extracellular component comprising an extracellular domain from human TGFβRI; (ii a transmembrane component comprising a transmembrane domain from human TGFβRI; and (iii) an intracellular component comprising a cytoplasmic/signaling domain from human IL-2Rγ;
(D) (i) an extracellular component comprising an extracellular domain from human TGFβRI; (ii) a transmembrane component comprising a transmembrane domain from human TGFβRI; and (iii) an intracellular component comprising a cytoplasmic/signaling domain from human IL-2Rγ, wherein the intracellular component further comprises a human TGFβR1 intracellular overhang or intracellular overhang sequence comprising, consisting essentially of, or consisting of the amino acid sequence C-H-N, disposed N-terminal to the human IL-2Rγ cytoplasmic/signaling domain;
(E) (i) an extracellular component comprising an extracellular domain from human TGFβRI; (ii) a transmembrane component comprising a transmembrane domain from human IL-2Rβ and (iii) an intracellular component comprising a cytoplasmic/signaling domain from human IL-2Rβ,
(F) (i) an extracellular component comprising an extracellular domain from human TGFβRI; (ii) a transmembrane component comprising a transmembrane domain from human IL-2Rγ, and (iii) an intracellular component comprising a cytoplasmic/signaling domain from IL-2Rγ;
(G) (i) an extracellular component comprising an extracellular domain from human TGFβRII; (ii) a transmembrane component comprising a transmembrane domain from human TGFβRII; and (iii) an intracellular component comprising a cytoplasmic/signaling domain from human IL-2Rβ;
(H) (i) an extracellular component comprising an extracellular domain from human TGFβRII; (ii) a transmembrane component comprising a transmembrane domain from human TGFβRII; and (iii) an intracellular component comprising a cytoplasmic/signaling domain from human IL-2Rβ, wherein the intracellular component further comprises a human TGFβRII intracellular overhang or intracellular overhang sequence comprising, consisting essentially of, or consisting of the amino acid sequence R-V-N, disposed N-terminal to the IL-2Rβ cytoplasmic/signaling domain;
(I) (i) an extracellular component comprising an extracellular domain from human TGFβRII; (ii) a transmembrane component comprising a transmembrane domain from human TGFβRII; and (iii) an intracellular component comprising a cytoplasmic/signaling domain from human IL-2Rγ;
(J) (i) an extracellular component comprising an extracellular domain from human TGFβRII; (ii) a transmembrane component comprising a transmembrane domain from human TGFβRII; and (iii) an intracellular component comprising a cytoplasmic/signaling domain from human IL-2Rγ, wherein the intracellular component further comprises a human TGFβRII intracellular overhang or intracellular overhang sequence comprising, consisting essentially of, or consisting of the amino acid sequence R-V-N, disposed N-terminal to the IL-2Rγ cytoplasmic/signaling domain;
(K) (i) an extracellular component comprising an extracellular domain from human TGFβRII; (ii) a transmembrane component comprising a transmembrane domain from human IL-2Rβ and (iii) an intracellular component comprising a cytoplasmic/signaling domain from human IL-2Rβ; or
(L) (i) an extracellular component comprising an extracellular domain from human TGFβRII; (ii) a transmembrane component comprising a transmembrane domain from human IL-2Rγ, and (iii) an intracellular component comprising a cytoplasmic/signaling domain from IL2Rγ.

26.-32. (canceled)

33. A fusion protein comprising or consisting of the amino acid sequence set forth in any one of SEQ ID NOs.: 5, 6, 7, 9, 10, 11, 13, 14, 15, 17, 18, and 19.

34. (canceled)

35. A polynucleotide encoding;
(1) the fusion protein of claim 1; or
(2) an amino acid sequence comprising the fusion protein of claim 1, and N-terminal to and connected to the extracellular component of the fusion protein, a signal peptide.

36. A polynucleotide encoding the fusion protein of claim 25.

37. A polynucleotide encoding the fusion protein of claim 33.

38. A polynucleotide encoding (1) a first fusion protein of claim 1 and (2) a second fusion protein of claim 1, wherein the first fusion protein and the second fusion protein are different from one another.

39. The polynucleotide of claim 38,
wherein: the first fusion protein and the second fusion protein comprise an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NOs: (i) 5 and 6, respectively; (ii) 5 and 9, respectively, (iii) 5 and 14, respectively; (iv) 5 and 17, respectively; (v) 10 and 6, respectively; (vi) 10 and 9, respectively; (vii) 10 and 14, respectively; (viii) 10 and 17, respectively; (ix) 13 and 6, respectively; (x) 13 and 9, respectively; (xi) 13 and 14, respectively; (xii) 13 and 17, respectively; (xiii) 18 and 6, respectively; (xiv) 18 and 9, respectively; (xv) 18 and 14, respectively; or (xvi) 18 and 17, respectively.

40.-41. (canceled)

42. A polynucleotide encoding: (1) a first fusion protein of claim 25 and (2) a second fusion protein of claim 25, wherein the first fusion protein and the second fusion protein are different from one another.

43. The polynucleotide of claim 42,
wherein: the first fusion protein and the second fusion protein are according to Table B.

44.-55. (canceled)

56. A vector comprising (1) the polynucleotide of claim 35 or (2) the polynucleotide of claim 35, wherein the polynucleotide is operably linked to an expression control sequence.

57.-61. (canceled)

62. A host cell expressing the fusion protein of claim 1, or comprising a polynucleotide encoding the fusion protein of claim 1, or comprising a vector comprising a polynucleotide encoding the fusion protein of claim 1.

63. A host cell expressing the fusion protein of claim 25, or comprising a polynucleotide encoding the fusion protein of claim 25, or comprising a vector comprising a polynucleotide encoding the fusion protein of claim 25.

64. (canceled)

65. A host cell expressing a first fusion protein and a second fusion protein, wherein:
(1) the first fusion protein and the second fusion protein comprise an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NOs: (i) 5 and 6, respectively; (ii) 5 and 9, respectively; (iii) 5 and 14, respectively; (iv) 5 and 17, respectively; (v) 10 and 6, respectively; (vi) 10 and 9, respectively; (vii) 10 and 14, respectively; (viii) 10 and 17, respectively; (ix) 13 and 6, respectively, (x) 13 and 9, respectively, (xi) 13 and 14, respectively; (xii) 13 and 17, respectively; (xiii) 18 and 6, respectively; (xiv) 18 and 9, respectively; (xv) 18 and 14, respectively: or (xvi) 18 and 17, respectively; and/or
(2) the first fusion protein and the second fusion protein are according to Table B.

66.-73. (canceled)

74. The host cell of claim 62, wherein the host cell comprises:
(1) a hematopoietic progenitor cell or a human immune system cell; and/or
(2) a T cell, a CD4+ T cell, a CD8+ T cell, a CD4−CD8− double negative T cell, or a γδ T cell, a natural killer cell, a natural killer T cell, a dendritic cell, or any combination thereof, and/or
(3) a T cell, wherein the T cell comprises a naïve T cell, a central memory T cell, a stem cell memory T cell, an effector memory T cell, or any combination thereof; and/or
(4) a chromosomal gene knockout or a mutation of: a TGFβR1 gene locus, a TGFβR2 gene locus, a PD-1 gene locus, a CTLA4 gene locus, a LAT gene locus, a TIM-3 gene locus, a PD-L1 gene locus, a TIGIT gene locus, an A2AR gene locus, a Fas locus, a FasL gene locus, a B7-H3 gene locus, a B7-H4 gene locus, an IDO gene locus, a VISTA gene locus, a SIGLEC7 gene locus, a SIGLEC9 gene locus, a TRAC gene locus, a TRBC gene locus, a T cell receptor gene locus, a MHC (e.g. HLA) gene locus, a CBLB gene locus, a RASA2 gene locus, a UBASH3A gene locus, a CISH gene locus, or any combination thereof, and/or
(5) a modification to have reduced protein expression (including null expression), of an endogenous TGFβR1, an endogenous TGFβR2, or both, as compared to the unmodified host cell; and/or
(6) a polynucleotide encoding an antigen-binding protein; and or
(7) a host cell which expresses a/the antigen-binding protein; and/or
(8) a polynucleotide encoding an antigen-binding protein, wherein the antigen-binding protein comprises a TCR, an antigen-binding fragment of a TCR, a scTv, a scTCR, a CAR, or any combination thereof; and/or
(9) a polynucleotide encoding an antigen-binding protein, wherein the antigen-binding protein binds to: a cancer antigen; an autoimmune antigen, a viral antigen, a bacterial antigen; a fungal antigen; a parasitic antigen; or any combination thereof; and/or
(10) a polynucleotide encoding an antigen-binding protein, wherein the antigen-binding protein binds to: a cancer antigen, and wherein the cancer comprises a solid tumor, a hematological malignancy, or both, and/or
(11) a polynucleotide encoding an antigen-binding protein, wherein the antigen is selected from a WT1, mesothelin, KRAS, ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, NY-ESO, 5T4, BCMA, FAP, Carbonic anhydrase 9, BRAF, α-fetoprotein, MAGE-A3, MAGE-A4, SSX-2, PRAME, HA-1, β2M, ETA, tyrosinase, NRAS, a CEA antigen, or a CEA antigen in complex with a MHC molecule.

75.-93. (canceled)

94. A composition comprising:
(i) the fusion protein of claim 1; and/or
(ii) a polynucleotide encoding the fusion protein of (i); and/or
(iii) a vector comprising the polynucleotide of (ii); and/or
(iv) a host cell expressing the fusion protein of (i), and a pharmaceutically acceptable carrier, excipient, or diluent.

95. (canceled)

96. A method of treating a disease or condition in a subject, the method comprising administering to the subject an effective amount of:
(i) the fusion protein of claim 1; and/or
(ii) a polynucleotide encoding the fusion protein of (i); and/or
(iii) a vector comprising the polynucleotide of (ii); and/or
(iv) a host cell expressing the fusion protein of (i); and/or
(v) a composition comprising any one of (i)-(iv), and/or
(vi) or any combination thereof.

97. A method of treating a disease or condition in a subject, the method comprising administering to the subject an effective amount of:
(i) the fusion protein of claim 25; and/or
(ii) a polynucleotide encoding the fusion protein of (i); and/or
(iii) a vector comprising the polynucleotide of (ii) and/or
(iv) a host cell expressing the fusion protein of (i); and/or
(v) a composition comprising any one of (i)-(iv), and/or
(vi) or any combination thereof.

98.-15. (canceled)

116. A method comprising introducing the polynucleotide of claim 35 or a vector comprising the polynucleotide of claim 35 into a host cell.

117. A method comprising introducing the polynucleotide of claim 36 or a vector comprising the polynucleotide of claim 36 into a host cell.


SEQUENCE LISTING (60 sequences)

SEQ ID NO: 1 - protein, 503 aa, Homo sapiens
  source 1..503: note = Human TGF-beta receptor type-1, isoform 1 (UniProt KB P36897-1), with signal peptide
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 2 - protein, 567 aa, Homo sapiens
  source 1..567: note = Human TGF-beta receptor type-2, isoform 1 (UniProt KB P37173-1), with signal peptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 3 - protein, 551 aa, Homo sapiens
  source 1..551: note = Human Interleukin-2 receptor subunit beta (UniProt KB P14784), with signal peptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 4 - protein, 369 aa, Homo sapiens
  source 1..369: note = Human Interleukin-2 receptor subunit gamma, isoform 1 (UniProt KB P31785-1), with signal peptide
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 448 aa, synthetic construct
  source 1..448: note = TGFbetaR2_IL-2Rbeta chimeric protein (with IL-2Rbeta transmembrane domain)
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 6 - protein, 207 aa, synthetic construct
  source 1..207: note = TGFbetaR1_IL-2Rgamme chimeric protein (with IL-2Rgamma transmembrane domain)
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 7 - protein, 737 aa, synthetic construct
  source 1..737: note = TGFbetaR2_IL-2Rbeta linked to TGFbetaR1_IL-2Rgamma by furin-linker-P2A cleavage sequence, with signal peptides
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVRAKRSGSGATNFSLLKQAGDVEENPGPMEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 8 - DNA, 2211 bp, synthetic construct
  source 1..2211: mol_type = other DNA; note = Polynucleotide encoding [TGFbetaR2_IL-2Rbeta linked to TGFbetaR1_IL-2Rgamma by furin-linker-P2A cleavage sequence, with signal peptides]
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggagaagaaaaagcctggtgagactttcttcatgtgttcctgtagctctgatgagtgcaatgacaacatcatcttctcagaagaatataacaccagcaatcctgacattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgcgggccaagcggtccggatccggagccacgaacttctctctgttaaagcaagcaggagacgtggaagaaaaccccggtcccatggaggcggcggtcgctgctccgcgtccccggctgctcctccttgtgcttgctgcagcggcagcagctgcagcggcacttctcccgggggcgacggcgttacagtgtttctgccacctctgtacaaaagacaattttacttgtgtgacagatgggctctgctttgtctctgtcacagagaccaccgacaaagtgatccacaacagcatgtgcatcgcggagatcgacctgattcctcgggaccgccccttcgtgtgtgcaccctcttcaaaaactgggtctgtgactacaacatattgctgcaatcaggaccattgcaataaaatagaacttccaactactgtaaagtcatcacctggccttggtcctgtggaacctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacc

SEQ ID NO: 9 - protein, 403 aa, synthetic construct
  source 1..403: note = TGFbetaR1_IL-2Rbeta chimeric protein (with IL-2Rbeta transmembrane domain)
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 10 - protein, 252 aa, synthetic construct
  source 1..252: note = TGFbetaR2_IL-2Rgamma chimeric protein (with IL-2Rgamma transmembrane domain)
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 11 - protein, 737 aa, synthetic construct
  source 1..737: note = TGFbetaR1_IL-2Rbeta linked to TGFbetaR2_IL-2Rgamma by furin-linker-P2A cleavage sequence, with signal peptides
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVRAKRSGSGATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 12 - DNA, 2211 bp, synthetic construct
  source 1..2211: mol_type = other DNA; note = Polynucleotide encoding [TGFbetaR1_IL-2Rbeta linked to TGFbetaR1_IL-2Rgamma by furin-linker-P2A cleavage sequence, with signal peptides]
atggaggcggcggtcgctgctccgcgtccccggctgctcctccttgtgcttgctgcagcggcagcagctgcagcggcacttctcccgggggcgacggcgttacagtgtttctgccacctctgtacaaaagacaattttacttgtgtgacagatgggctctgctttgtctctgtcacagagaccaccgacaaagtgatccacaacagcatgtgcatcgcggagatcgacctgattcctcgggaccgccccttcgtgtgtgcaccctcttcaaaaactgggtctgtgactacaacatattgctgcaatcaggaccattgcaataaaatagaacttccaactactgtaaagtcatcacctggccttggtcctgtggaaattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgcgggccaagcggtccggatccggagccacgaacttctctctgttaaagcaagcaggagacgtggaagaaaaccccggtcccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggagaagaaaaagcctggtgagactttcttcatgtgttcctgtagctctgatgagtgcaatgacaacatcatcttctcagaagaatataacaccagcaatcctgaccctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacc

SEQ ID NO: 13 - protein, 453 aa, synthetic construct
  source 1..453: note = TGFbetaR2-TM_IL-2Rbeta chimeric protein
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 14 - protein, 203 aa, synthetic construct
  source 1..203: note = TGFbetaR1-TM-with intracellular overhang_IL-2Rgamm chimeric protein
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 15 - protein, 738 aa, synthetic construct
  source 1..738: note = TGFbetaR2-TM_IL-2Rbeta linked to TGFbetaR1-TM-with intracellular overhang_IL-2Rgamma by furin-linker-P2A cleavage sequence, with signal peptides
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVRAKRSGSGATNFSLLKQAGDVEENPGPMEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 16 - DNA, 2214 bp, synthetic construct
  source 1..2214: mol_type = other DNA; note = Polynucleotide encoding [TGFbetaR2-TM_IL-2Rbeta linked to TGFbetaR1-TM-with intracellular overhang_IL-2Rgamma by furin-linker-P2A cleavage sequence, with signal peptides]
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggagaagaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctacaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgcgggccaagcggtccggatccggagccacgaacttctctctgttaaagcaagcaggagacgtggaagaaaaccccggtcccatggaggcggcggtcgctgctccgcgtccccggctgctcctccttgtgcttgctgcagcggcagcagctgcagcggcacttctcccgggggcgacggcgttacagtgtttctgccacctctgtacaaaagacaattttacttgtgtgacagatgggctctgctttgtctctgtcacagagaccaccgacaaagtgatccacaacagcatgtgcatcgcggagatcgacctgattcctcgggaccgccccttcgtgtgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaacgaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacc

SEQ ID NO: 17 - protein, 403 aa, synthetic construct
  source 1..403: note = TGFbetaR1-TM +3aa_IL2Rbeta chimeric protein
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 18 - protein, 253 aa, synthetic construct
  source 1..253: note = TGFbetaR2-TM-without intracellular overhang_IL2Rgamma chimeric protein
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 19 - protein, 738 aa, synthetic construct
  source 1..738: note = TGFbetaR1-TM-with intracellular overhang_IL-2Rbeta linked to TGFbetaR2-TM _IL-2Rgamma by furin-linker-P2A cleavage sequence, with signal peptides
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVRAKRSGSGATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 20 - DNA, 2214 bp, synthetic construct
  source 1..2214: mol_type = other DNA; note = Polynucleotide encoding [TGFbetaR1-TM-with intracellular overhang _IL-2Rbeta linked to TGFbetaR2-TM _IL-2Rgamma by furin-linker-P2A cleavage sequence, with signal peptides]
atggaggcggcggtcgctgctccgcgtccccggctgctcctccttgtgcttgctgcagcggcagcagctgcagcggcacttctcccgggggcgacggcgttacagtgtttctgccacctctgtacaaaagacaattttacttgtgtgacagatgggctctgctttgtctctgtcacagagaccaccgacaaagtgatccacaacagcatgtgcatcgcggagatcgacctgattcctcgggaccgccccttcgtgtgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaacaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgcgggccaagcggtccggatccggagccacgaacttctctctgttaaagcaagcaggagacgtggaagaaaaccccggtcccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggagaagaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctacgaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacc

SEQ ID NO: 21 - protein, 33 aa, synthetic construct
  source 1..33: note = Signal peptide, TGFbetaR1
MEAAVAAPRPRLLLLVLAAAAAAAAALLPGATA

SEQ ID NO: 22 - protein, 22 aa, synthetic construct
  source 1..22: note = Signal peptide, TGFbetaR2
MGRGLLRGLWPLHIVLWTRIAS

SEQ ID NO: 23 - skipped

SEQ ID NO: 24 - protein, 22 aa, synthetic construct
  source 1..22: note = Porcine teschovirus-1 2A (P2A) peptide with N-terminal GSG linker
GSGATNFSLLKQAGDVEENPGP

SEQ ID NO: 25 - protein, 24 aa, synthetic construct
  source 1..24: note = Thoseaasigna virus 2A (T2A) peptide
LEGGGEGRGSLLTCGDVEENPGPR

SEQ ID NO: 26 - protein, 20 aa, synthetic construct
  source 1..20: note = Equine rhinitis A virus (ERAV) 2A (E2A)
QCTNYALLKLAGDVESNPGP

SEQ ID NO: 27 - protein, 25 aa, synthetic construct
  source 1..25: note = Foot-and-Mouth disease virus 2A (F2A) peptide with N-terminal GSG linker
GSGVKQTLNFDLLKLAGDVESNPGP

SEQ ID NO: 28 - protein, 27 aa, synthetic construct
  source 1..27: note = Furin cleavage motif-serine-GSG-P2A
RAKRSGSGATNFSLLKQAGDVEENPGP

SEQ ID NO: 29 - protein, 277 aa, synthetic construct [sequence omitted]

SEQ ID NO: 30 - protein, 335 aa, synthetic construct
  source 1..335: note = Truncated EGFR (tEGFR)
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 31 - protein, 18 aa, synthetic construct
  source 1..18: note = Linker 218 amino acid sequence
GSTSGSGKPGSGEGSTKG

SEQ ID NO: 32 - protein, 10 aa, synthetic construct
  source 1..10: note = linker amino acid sequence
GSGKPGSGEG

SEQ ID NO: 33 - protein, 8 aa, synthetic construct
  source 1..8: note = linker amino acid sequence
GKPGSGEG

SEQ ID NO: 34 - protein, 8 aa, synthetic construct
  source 1..8: note = linker amino acid sequence
SGKPGSGE

SEQ ID NO: 35 - skipped

SEQ ID NO: 36 - protein, 110 aa, synthetic construct
  source 1..110: note = linker amino acid sequence
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS

SEQ ID NO: 37 - protein, 15 aa, synthetic construct
  source 1..15: note = linker amino acid sequence
GGGGSGGGGSGGGGS

SEQ ID NO: 38 - protein, 18 aa, synthetic construct
  source 1..18: note = linker amino acid sequence
GSTSGGGSGGGSGGGGSS

SEQ ID NO: 39 - protein, 92 aa, synthetic construct
  source 1..92: note = Human TGFbetaR1 extracellular domain
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 40 - protein, 22 aa, synthetic construct
  source 1..22: note = Human TGFbetaR1 transmembrane domain
LAAVIAGPVCFVCISLMLMVYI

SEQ ID NO: 41 - protein, 137 aa, synthetic construct
  source 1..137: note = Human TGFbetaR2 extracellular domain
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 42 - protein, 30 aa, synthetic construct
LLLVIFQVTGISLLPPLGVAISVIIIFYCY

SEQ ID NO: 43 - protein, 25 aa, synthetic construct
  source 1..25: note = Human IL-2Rbeta transmembrane domain
IPWLGHLLVGLSGAFGFIILVYLLI

SEQ ID NO: 44 - protein, 286 aa, synthetic construct
  source 1..286: note = Human IL-2Rbeta cytoplasmic/signaling domain
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 45 - protein, 29 aa, synthetic construct
  source 1..29: note = Human IL-2Rgamma transmembrane domain
PFLFALEAVVISVGSMGLIISLLCVYFWL

SEQ ID NO: 46 - protein, 86 aa, synthetic construct
  source 1..86: note = Human IL-2Rgamma cytoplasmic/signaling domain
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 47 - protein, 10 aa, synthetic construct
  source 1..10: note = Sequence comprising TGFbetaR1 partial extracellular and domain and transmembrane domain amino acids
SSPGLGPVEL

SEQ ID NO: 48 - protein, 14 aa, synthetic construct
  source 1..14: note = Sequence comprising TGFbetaR1 partial extracellular and domain and transmembrane domain amino acids
IFSEEYNTSNPDLL

SEQ ID NO: 49 - DNA, 20 bp, synthetic construct
  source 1..20: mol_type = other DNA; note = sgTGFbetaRI guide RNA
catacaaacggcctatctcg

SEQ ID NO: 50 - DNA, 20 bp, synthetic construct
  source 1..20: mol_type = other DNA; note = sgTGFbetaRII guide RNA
tcacccgacttctgaacgtg

SEQ ID NO: 51 - DNA, 20 bp, synthetic construct
  source 1..20: mol_type = other DNA; note = TGFbetaRI fwd genomic primer
agtgtttctgccacctctgt

SEQ ID NO: 52 - DNA, 20 bp, synthetic construct
  source 1..20: mol_type = other DNA
tgcctctaaacggaatgagc

SEQ ID NO: 53 - skipped

SEQ ID NO: 54 - protein, 4 aa, synthetic construct
  source 1..4: note = Furin cleavage sequence
RAKR

SEQ ID NO: 55 - protein, 4 aa, synthetic construct
  source 1..4: note = Furin cleavage sequence
RARR

SEQ ID NO: 56 - protein, 25 aa, synthetic construct
  source 1..25: note = TGFbetaRI transmembrane sequence with 3-amino acid intracellular overhang (CHN)
LAAVIAGPVCFVCISLMLMVYICHN

SEQ ID NO: 57 - DNA, 20 bp, synthetic construct
  source 1..20: mol_type = other DNA; note = sgTGFbetaRII guide RNA
ctagtcatatttcaagtgac

SEQ ID NO: 58 - protein, 1380 aa, synthetic construct
  source 1..1380: note = Msln-specific TCR with murinized TGFbRI-IL2b-furin-GSG-P2A-TGFbRII-IL2g, with signal peptides
MTLKMDSSPGFVAVILLILGRTHGDSVTQTEGPVTVSESESLIINCTYSATSIAYPNLFWYVRYPGEGLQLLLKVITAGQKGSSRGFEATYNKETTSFHLQKASVQESDSAVYYCALGLDYANKMIFGLGTILRVRPHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMEAAAAAPRRPQLLIVLVAAATLLPGAKALQCFCHLCTKDNFTCETDGLCFVSVTETTDKVIHNSMCIAEIDLIPRDRPFVCAPSSKTGAVTTTYCCNQDHCNKIELPTTGPFSEKQSAGLGPVEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLIRAKRGSGATNFSLLKQAGDVEENPGPMGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEARAKRSGSGQCTNYALLKLAGDVESNPGPMDPRLLCCVIFCLLAATFVDTTVKQNPRYKLARVGKPVNLICSQTMNHDTMYWYQKKPNQAPKLLLFYYDKILNREADTFEKFQSSRPNNSFCSLYIGSAGLEYSAMYLCASSNPQDTQYFGPGTRLLVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 59 - protein, 1375 aa, synthetic construct
MTLKMDSSPGFVAVILLILGRTHGDSVTQTEGPVTVSESESLIINCTYSATSIAYPNLFWYVRYPGEGLQLLLKVITAGQKGSSRGFEATYNKETTSFHLQKASVQESDSAVYYCALGLDYANKMIFGLGTILRVRPHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDLLLVIIQVTGVSLLPPLGIAIAVIIIFYCYKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLIRAKRGSGATNFSLLKQAGDVEENPGPMEAAAAAPRRPQLLIVLVAAATLLPGAKALQCFCHLCTKDNFTCETDGLCFVSVTETTDKVIHNSMCIAEIDLIPRDRPFVCAPSSKTGAVTTTYCCNQDHCNKIELPTTGPFSEKQSAGLGPVELAAVIAGPVCFVCIALMLMVYIERMPPIPPIKNLEDLVTEYQGNFSAWSGVSKGLTESLQPDYSERFCHVSEIPPKGGALGEGPGGSPCSLHSPYWPPPCYSLKPEARAKRSGSGQCTNYALLKLAGDVESNPGPMDPRLLCCVIFCLLAATFVDTTVKQNPRYKLARVGKPVNLICSQTMNHDTMYWYQKKPNQAPKLLLFYYDKILNREADTFEKFQSSRPNNSFCSLYIGSAGLEYSAMYLCASSNPQDTQYFGPGTRLLVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 60 - protein, 33 aa, synthetic construct
LLLVIFQVTGISLLPPLGVAISVIIIFYCYRVN